SEQUENCE LISTING (30 sequences)

SEQ ID NO: 1 - DNA, 6890 bp, Artificial Sequence
  hSYN1-GlyRalpha1 F207A/A288G gene therapy vector
gacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccaggactctagctatagttctagtggttggctacagcttgctgcgcgctcgctcgctcactgaggccgcccgggcaaagcccgggcgtcgggcgacctttggtcgcccggcctcagtgagcgagcgagcgcgcagagagggagtggggttgatctctccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagtcgacggtatcgatttaaaccttatcgtcgtcatccttgtaatcgagcggccgcgtacgcgtctggttgtggacgtcctctctacggacaatcttgtagatgatccagtagaacatgttgaaaatgaggaaggccatggggaagccaatgcgggatattttgtcgatcttcttggccctctggatgaagagttttcgcatctcctctggggacttagatggtgcaggaggggggttggtggtgttactgttgttggcgcccttgactgagatgccatccttggcctgtagacaggctgggcccatcccataggcagagaagttaaagcggccttctccagcttcatcctcctggaatagattcaacatggggctcttgtgatgtctccgcttcctcctgaatcggagcagctccttatgttgccgagacacaaagttaacggcagcatattctaatagggctgagaacacaaagagcaggcaaactcccatccaaatgtcaatggctttcacataggacaccttgggcagagatgctcgagagccggagctctgggtggtcatggtgagcacagtggtgatgcctaggcccacacgagcaggtgcagcatccatgttgatccagaaggagatccatgagaggatgacaatgagcaggctgggaatatacatctgaatcaggtagtaacccatctgccgctccaggtggaaccgggcctcaatgcaggtggctttacctgtgttgtagtgcttggtgcagtatctcaagtccttctcttccttcaagataaactggggcagagttagtccatctgctacctgcacggctccctgttcctgccactcaaagatgaggtcattcatcgtatatccaaagctttccagttgcatgatacatgtctggacatccatggggaaattcttcaagtccatggggcaggccagtgtcagggtgattctgatgctgtagaggacattcccattccgggagatccttagcaatttgttgtctgtggtgatctcatggaagtgggcccccttctcgttggcaaagaacaggtcaggtttccagatggagtccagcatggatgggtccaggtccagagagtcgtcagggtattcattataggccaggcgggggtcgttccattgctgccgcaggaagatgttgaccctatagtccatggttgtctcagcaatggaaccaaagctgttgatgaaaatgttgcagctcacgttcactgggggacctttaaaattgggcctgatcctggcatcatatccggaggttctccccattagcttatccaggaaatccgagggtgacataggcttgggtgcggagcgagccatggtggctagcagcttgaattctcgactgcgctctcaggcacgacacgactcctccgctgcccaccgcagactgaggcagcgctgagtcgccggcgccgcagcgcagatggtcgcgcccgtgcccccctatctcgcgcctcgcgtggtgcggtccggctgggccggcggcggcgcggacgcgaccaaggtggccgggaaggggagtttgcgggggaccggcgagtgacgtcagcgcgccttcagtgctgaggcggcggtggcgcgcgccgccaggcgggggcgaaggcactgtccgcggtgctgaagctggcagtgcgcacgcgcctcgccgcatcctgtttcccctccccctctctgataggggatgcgcaatttggggaatgggggttgggtgcttgtccagtgggtcggggtcggtcgtcaggtaggcacccccaccccgcctcatcctggtcctaaaacccacttgcactcatacgcagggccctctgcagtctagtggtaccgaattcagatctgcagcttcctaggaacccctagtgatggagttggccactccctctctgcgcgctcgctcgctcactgaggccgggcgaccaaaggtcgcccgacgcccgggctttgcccgggcggcctcagtgagcgagcgagcgcgccaggatccgagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttgcagcctgaatggcgaatg

SEQ ID NO: 2 - DNA, 10 bp, Unknown
  Kozak sequence
gccrccatgg

SEQ ID NO: 3 - protein, 5 aa, Unknown
  linker sequence
DGGGS

SEQ ID NO: 4 - protein, 5 aa, Unknown
  linker sequence
TGEKP

SEQ ID NO: 5 - protein, 4 aa, Unknown
  linker sequence
GGRR

SEQ ID NO: 6 - protein, 5 aa, Unknown
  linker sequence
GGGGS

SEQ ID NO: 7 - protein, 14 aa, Unknown
  linker sequence
EGKSSGSGSESKVD

SEQ ID NO: 8 - protein, 18 aa, Unknown
  linker sequence
KESGSVSSEQLAQFRSLD

SEQ ID NO: 9 - protein, 8 aa, Unknown
  linker sequence
GGRRGGGS

SEQ ID NO: 10 - protein, 9 aa, Unknown
  linker sequence
LRQRDGERP

SEQ ID NO: 11 - protein, 12 aa, Unknown
  linker sequence
LRQKDGGGSERP

SEQ ID NO: 12 - protein, 16 aa, Unknown
  linker sequence
LRQKDGGGSGGGSERP

SEQ ID NO: 13 - protein, 7 aa, Tobacco etch virus
  misc_feature (2)..(3): Xaa can be any naturally occurring amino acid
  misc_feature (5)..(5): Xaa can be any naturally occurring amino acid
  MISC_FEATURE (7)..(7): Xaa may be Gly or Ser
EXXYXQX

SEQ ID NO: 14 - protein, 7 aa, Tobacco etch virus
ENLYFQG

SEQ ID NO: 15 - protein, 7 aa, Tobacco etch virus
ENLYFQS

SEQ ID NO: 16 - DNA, 20 bp, Artificial Sequence
  2793F primer
ataggaccctgcaggtatac

SEQ ID NO: 17 - DNA, 30 bp, Artificial Sequence
  2794R primer
cgtttctaaagtaaaaacagacaacaacaa

SEQ ID NO: 18 - DNA, 30 bp, Artificial Sequence
  2795F primer
ctgtttttactttagaaacgcgctgctgcc

SEQ ID NO: 19 - DNA, 20 bp, Artificial Sequence
  2796R primer
gtaccagttgccgtacgtcc

SEQ ID NO: 20 - DNA, 30 bp, Artificial Sequence
  2799F primer
gatctggtaccactagactgcagagggccc

SEQ ID NO: 21 - DNA, 40 bp, Artificial Sequence
  2800R primer
gtggctagcagcttgaattctcgactgcgctctcaggcac

SEQ ID NO: 22 - DNA, 40 bp, Artificial Sequence
  2801F primer
gaattcaagctgctagccaccatggctcgctccgcaccca

SEQ ID NO: 23 - DNA, 30 bp, Artificial Sequence
  2802R primer
tgcaggtggctttacctgtgttgtagtgct

SEQ ID NO: 24 - DNA, 30 bp, Artificial Sequence
  2803F primer
cacaggtaaagccacctgcattgaggcccg

SEQ ID NO: 25 - DNA, 30 bp, Artificial Sequence
  2804R primer
gcaggcaaactcccatccaaatgtcaatgg

SEQ ID NO: 26 - DNA, 30 bp, Artificial Sequence
  2805F primer
ggatgggagtttgcctgctctttgtgttct

SEQ ID NO: 27 - DNA, 50 bp, Artificial Sequence
  2806R primer
atccttgtaatcgagcggccgcgtacgcgtctggttgtggacgtcctctc

SEQ ID NO: 28 - DNA, 50 bp, Artificial Sequence
  2807F primer
ggccgctcgattacaaggatgacgacgataaggtttaaatcgataccgtc

SEQ ID NO: 29 - DNA, 50 bp, Artificial Sequence
  2808F primer
aaggtttaaatcgataccgtcgactagagctcgctgatcagcctcgactg

SEQ ID NO: 30 - DNA, 30 bp, Artificial Sequence
  2809R primer
cccagcatgcctgctattgtcttcccaatc

(19) United States
(12) Patent Application Publication
(10) Pub. No.: US 2018/0193414 A1
(43) Pub. Date: Jul. 12, 2018

(54) COMPOSITIONS AND METHODS FOR TREATING NEUROLOGICAL DISORDERS

(71) Applicant: Coda Biotherapeutics, Inc., South San Francisco, CA (US)

(72) Inventors: Kenneth P. GREENBERG, Berkeley, CA (US); Nathaniel E. DAVID, San Francisco, CA (US); Mitchell H. FINER, Stow, MA (US)

(21) Appl. No.: 15/894,633

(22) Filed: Feb. 12, 2018

Related U.S. Application Data
(63) Continuation of application No. PCT/US2016/052384, filed on Sep. 17, 2016.
(60) Provisional application No. 62/220,077, filed on Sep. 17, 2015.
(60) Provisional application No. 62/220,087, filed on Sep. 17, 2015.

(51) Int. Cl. A61K 38/17; A61K 31/366; C07K 14/705; C12N 15/861; A61K 9/00; A61P 25/28; A61P 25/16

(52) U.S. Cl. CPC A61K 38/177; A61K 31/366; C07K 14/705; A61K 2300/00; A61K 9/0019; A61P 25/28; A61P 25/16; C12N 15/861

(57) ABSTRACT

The present invention generally provides vectors, compositions, and methods of using the same for treating neurological disorders, including managing pain. The compositions and methods include the use of G protein-coupled receptors and ligand-gated ion channels to treat neurological indications including pain, epilepsy and satiety disorders. The compositions and methods further include the use of synthetic ligands to activate the G protein-coupled receptors and ligand-gated ion channels in the treatment of neurological disease.

### CROSS-REFERENCE TO RELATED APPLICATIONS

This application is a bypass continuation, under 35 U.S.C. § 111(a), claiming priority to International Patent Application No. PCT/US2016/052384, filed Sep. 17, 2016, which claims priority to and benefit of U.S. Provisional Patent Application No. 62/220,077, filed on Sep. 17, 2015 and U.S. Provisional Patent Application No. 62/220,087, filed on Sep. 17, 2015. The contents of these applications are herein incorporated by reference in their entireties.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: SWCH_004_01US_SeqList_ST25.txt, date recorded: Dec. 12, 2017, file size 15 kilobytes).

### TECHNICAL FIELD

The present invention generally relates to viral vectors encoding receptors, compositions, and related methods of use for treating neurological disorders, including managing pain.

### BACKGROUND OF THE INVENTION

Hundreds of millions of people worldwide are affected by neurological disorders. It is currently estimated that there are over 600 different neurological disorders that affect people. Approximately 6.2 million people die because of stroke each year with over 80% of deaths in low- and middle-income countries. More than 50 million people suffer from epilepsy worldwide. It is estimated that there are globally 35.6 million people with dementia with 7.7 million new cases every year. Alzheimer's disease is the most common cause of dementia and may contribute to 60-70% of cases. The prevalence of migraine is more than 10% worldwide. More than 110 million Americans alone suffer from chronic pain. The financial burden of neurological disorders is significant. In the United States alone, the cost to treat neurological disorders is estimated to be over $800 billion a year. The global cost of treating neurological disorders is estimated to exceed $6 trillion by the year 2030.

Chronic pain is one type of neurological disorder. Unrelieved chronic pain is a critical health problem in the US and worldwide. A report by the Institute of Medicine estimated that 116 million Americans suffer from pain that persists for weeks to years, with resulting annual costs exceeding $560 million. There are no adequate long-term therapies for chronic pain sufferers, leading to significant cost for both society and the individual. Pain often results in disability and, even when not disabling, it has a profound effect on the quality of life. Pain treatment frequently fails even when the circumstances of care delivery are optimal, such as attentive, well-trained physicians; ready access to opioids; use of adjuvant analgesics; availability of patient-controlled analgesia; and evidence-based use of procedures like nerve blocks and IT pumps.

The most commonly used therapy for chronic pain is the application of opioid analgesics and nonsteroidal anti-inflammatory drugs, but these drugs can lead to addiction and may cause side effects, such as drug dependence, tolerance, respiratory depression, sedation, cognitive failure, hallucinations, and other systemic side effects. Despite the wide usage of pharmaceuticals, there is a strikingly low success rate for its effectiveness in pain relief. A large randomized study with various medications found only one out of every two or three patients achieving at least 50% pain relief (Finnerup et al., 2005). A follow-up study using the most developed pharmacological treatments found the same results, indicating that there was no improvement in the efficacy of medications for pain (Finnerup et al., Pain, 150(3):573-81, 2010).

More invasive options for the treatment of pain include nerve blocks and electrical stimulation. A nerve block is a local anesthetic injection usually in the spinal cord to interrupt pain signals to the brain, the effect of which only lasts from weeks to months. Nerve blocks are not the recommended treatment option in most cases (Mailis and Taenzer, Pain Res Manag. 17(3):150-158, 2012). Electrical stimulation involves providing electric currents to block pain signals. Although the effect may last longer than a nerve block, complications arise with the electrical leads itself: dislocation, infection, breakage, or the battery dying. One review found that 40% of patients treated with electrical stimulation for neuropathy experienced one or more of these issues with the device (Wolter, 2014).

The most invasive, and least preferred, method for managing pain is complete surgical removal of the nerve or section thereof that is causing the pain. This option is only recommended when the patient has exhausted the former and other less invasive, treatments and found them ineffective. Radiofrequency nerve ablation uses heat to destroy problematic nerves and provides a longer pain relief than a nerve block. However, one study found no difference between the control and treatment groups in partial radiofrequency lesioning of the DRG for chronic lumbosacral radicular pain (Geurts et al., 2003). Other surgical methods for surgically removing the pain nerves suffer from similar shortcomings and have serious side effects long-term, including sensory or motor deficits, or cause pain elsewhere.

Methods for treating neurological disorders should be safe, efficient and cost-effective. Gene therapy could provide non-invasive treatment options for a variety of neurological diseases, including managing pain. However, to date, gene therapy methods have not found widespread use in the treatment of neurological diseases. The key to gene therapy is selecting safe and highly efficient gene delivery systems that can deliver therapeutic genes to overexpress or suppress relevant targets in specific cell types.

However, few delivery systems have been shown to be safe and efficient; thus, the promise of gene therapy for treating neurological disorders, including managing pain, has yet to be realized.

### SUMMARY OF THE INVENTION

The present invention provides polynucleotides, vectors, and related compositions for use in the gene therapy of neurological disorders and diseases. In one embodiment, the neurological disorder is pain (e.g., chronic or acute pain).

In one aspect, a method is provided for treating a neurological disease comprising administering a biologically inert agent or a drug to a subject suffering from the neurological disease. In some embodiments, the neurological disease is not epilepsy. In some cases, the subject heterologously expresses a G protein-coupled receptor or a ligand-gated ion channel (LGIC). In some cases, the subject homologously expresses a G protein-coupled receptor or an LGIC. In some cases, the subject ectopically expresses a G protein-coupled receptor or an LGIC. In some cases, the G protein-coupled receptor is a Designer Receptor Exclusively Activated by a Designer Drug (DREADD). In some examples, the DREADD is hM4Di, hM3Dq, AlstR or KOR-DREADD. In some cases, the LGIC is GlyR-M, GluCl, PSAM-5HT3HC, PSAM-GlyR, PSAM-nAChR, TRPV1 or GABAA. In some instances, the biologically inert agent is clozapine-N-oxide (CNO), nalfurafine (C₂₈H₃₂N₂O₅), salvinorin B, allatostatin, 8-Chloro-11-[4-(1,1-dideutrioethyl)piperazin-1-yl]-5H-dibenzo[b,e][1,4]diazepine or 11-(Piperazin-1-yl)-5H-dibenzo[b,e][1,4]diazepine. In some cases, the drug is ivermectin, selamectin, doramectin, emamectin, eprinomectin, abamectin, moxidectin, PSEM^{22S}, PSEM^{89S}, PSEM^{9S}, capsaicin, or zolpidem. In some embodiments, a method is provided for treating a neurological disease that is not epilepsy, comprising administering a biologically inert agent that is clozapine-N-oxide to a subject suffering from said neurological disease, wherein said subject expresses hM4Di. In some cases, the G protein-coupled receptor or LGIC is activated by the biologically inert agent or the drug. In some cases, the G protein-coupled receptor or LGIC is a switch receptor. In some cases, the method further comprises, prior to said administering, delivering a nucleic acid molecule encoding the G protein-coupled receptor or the LGIC to the subject. In some cases, the nucleic acid molecule is delivered to the subject in a viral vector. In some cases, the viral vector is an adenoviral vector, an adeno-associated viral (AAV) vector, a lentiviral vector, or a Herpes Simplex viral (HSV) vector. In some examples, the AAV vector is derived from AAV-6 or AAV-9. In some examples, the AAV vector is AAV6(Y705+731F+T492V), AAV9(Y731F) or AAV-7m8. In some embodiments, the AAV vector comprises SEQ ID NO:1. In some cases, the nucleic acid molecule is delivered to the subject by a non-viral method. In some examples, the non-viral method is lipofection, nanoparticle delivery, particle bombardment, electroporation, sonication or microinjection. In some cases, the neurological disease is pain. In some cases, the neurological disease is a satiety disorder. In some instances, the satiety disorder is obesity, anorexia nervosa or bulimia nervosa. In some cases, the neurological disease is Alzheimer's disease, Parkinson's disease, post-traumatic stress disorder (PTSD), gastroesophageal reflux disease (GERD), addiction, anxiety, depression, memory loss, dementia, sleep apnea, stroke, urinary incontinence, narcolepsy, essential tremor, movement disorder, atrial fibrillation or brain cancer. In some cases, the G protein-coupled receptor is Gᵢ- or G_{q}-coupled. In some cases, the G protein-coupled receptor or the LGIC is selectively expressed in an excitable cell. In some cases, the excitable cell is a neuron or a myocyte. In some cases, the neuron is a dorsal root ganglion or a sensory neuron. In some cases, the administering comprises oral, intrathecal, or topical administration. In some cases, the delivering comprises intrathecal, intraganglionic, intracranial, subcutaneous, intraspinal, cisterna magna or intraneural delivery. In some cases, the biologically inert agent or drug is administered at least one week after said delivering. In some cases, the biologically inert agent or drug is administered at a dose of 0.001 μg/kg to 10 mg/kg. In some cases, the nucleic acid molecule comprises a synapsin, TRPV1, Naᵥ1.7, Naᵥ1.8, Naᵥ1.9, CamKII, NSE or Advillin promoter. In some cases, the subject is a human. In some cases, the subject is a veterinary animal.

In another aspect, a method is provided for treating a neurological disease, the method comprising administering to a subject that heterologously expresses a G protein-coupled receptor or an LGIC, a drug that activates the G protein-coupled receptor or the LGIC, wherein the drug is a biologically inert agent or a synthetic ligand. In some aspects, the neurological disease is not epilepsy. In some cases, the G protein-coupled receptor is a DREADD. In some cases, the DREADD is hM4Di, hM3Dq, AlstR or KOR-DREADD. In some cases, the LGIC is GlyR-M, GluCl, PSAM-5HT3HC, PSAM-GlyR, PSAM-nAChR, TRPV1 or GABAA. In some cases, the G protein-coupled receptor or the LGIC is a switch receptor. In some cases, the biologically inert agent is clozapine-N-oxide, nalfurafine (C₂₈H₃₂N₂O₅), salvinorin B, allatostatin, 8-Chloro-11-[4-(1,1-dideutrioethyl)piperazin-1-yl]-5H-dibenzo[b,e][1,4]diazepine or 11-(Piperazin-1-yl)-5H-dibenzo[b,e][1,4]diazepine. In some cases, the drug is ivermectin, selamectin, doramectin, emamectin, eprinomectin, abamectin, moxidectin, PSEM^{22S}, PSEM^{89S}, PSEM^{9S}, capsaicin, or zolpidem. In some cases, the administering comprises oral, intrathecal or topical administration. In some cases, the method further comprises, prior to the administering, delivering to the subject a nucleic acid molecule encoding the G protein-coupled receptor or the LGIC. In some cases, the nucleic acid molecule encoding the G protein-coupled receptor or the LGIC is delivered by a viral vector. In some cases, the viral vector is an adenoviral vector, an adeno-associated viral (AAV) vector, a lentiviral vector or a Herpes Simplex viral (HSV) vector. In some examples, the AAV vector is derived from AAV-6 or AAV-9. In some examples, the AAV vector is AAV6(Y705+731F+T492V), AAV9(Y731F) or AAV-7m8. In some embodiments, the AAV vector comprises SEQ ID NO: 1. In some cases, the nucleic acid molecule is delivered to the subject by a non-viral method. In some cases, the non-viral method is lipofection, nanoparticle delivery, particle bombardment, electroporation, sonication or microinjection. In some cases, the neurological disease is pain. In some cases, the neurological disease is a satiety disorder. In some examples, the satiety disorder is obesity, anorexia nervosa or bulimia nervosa. In other cases, the neurological disease is Alzheimer's disease, Parkinson's disease, post-traumatic stress disorder (PTSD), gastroesophageal reflux disease (GERD), addiction, anxiety, depression, memory loss, dementia, sleep apnea, stroke, narcolepsy, urinary incontinence, essential tremor, movement disorder, atrial fibrillation or brain cancer. In some cases, the nucleic acid molecule comprises a synapsin, TRPV1, Naᵥ1.7, Naᵥ1.8, Naᵥ1.9, CamKII, NSE or Advillin promoter.

In another aspect, a method is provided for the treatment of neurological disease, the method comprising: administering to a subject heterologously expressing a G protein-coupled receptor or an LGIC, a drug that activates the G protein-coupled receptor or the LGIC, wherein the drug is not an endogenous ligand for the G protein-coupled receptor or the LGIC. In some cases, the neurological disease is not epilepsy. In some cases, the G protein-coupled receptor is hM4Di, hM3Dq, AlstR or KOR-DREADD. In some cases, the LGIC is GlyR-M, GluCl, PSAM-5HT3HC, PSAM-GlyR, PSAM-nAChR, TRPV1 or GABAA. In some cases, the drug is clozapine-N-oxide, nalfurafine (C₂₈H₃₂N₂O₅), salvinorin B, allatostatin, clozapine, olanzapine, perlapine, fluperlapine, alosetron, 8-Chloro-11-[4-(1,1-dideutrioethyl)piperazin-1-yl]-5H-dibenzo[b,e][1,4]diazepine or 11-(Piperazin-1-yl)-5H-dibenzo[b,e][1,4]diazepine. In some cases, the drug is ivermectin, selamectin, doramectin, emamectin, eprinomectin, abamectin, moxidectin, PSEM^{22S}, PSEM^{89S}, PSEM^{9S}, capsaicin, or zolpidem. In some cases, the method further comprises, prior to the administering, delivering a nucleic acid molecule encoding the G protein-coupled receptor or the LGIC to the subject. In some cases, the G protein-coupled receptor or the LGIC is selectively expressed in an excitable cell. In some cases, the excitable cell comprises a neuron or a myocyte. In some cases, the neuron comprises a sensory neuron, dorsal root ganglion or trigeminal ganglion. In some cases, the nucleic acid molecule is delivered by a viral vector. In some examples, the viral vector is an adenoviral vector, a lentiviral vector or an adeno-associated viral (AAV) vector. In some instances, the AAV vector is derived from AAV-6 or AAV-9. In some instances, the AAV vector is AAV6(Y705+731F+T492V), AAV9(Y731F) or AAV-7m8. In some cases, the nucleic acid molecule is delivered to the subject by a non-viral method. In some cases, the non-viral method is lipofection, nanoparticle delivery, particle bombardment, electroporation, sonication or microinjection. In some cases, the drug is a synthetic ligand. In some cases, the drug is administered at a dose of 0.001 μg/kg to 10 mg/kg. In some cases, the neurological disease is Alzheimer's disease, Parkinson's disease, pain, obesity, anorexia, PTSD, GERD, addiction, anxiety, depression, memory loss, dementia, sleep apnea, stroke, narcolepsy, urinary incontinence, essential tremor, movement disorder, atrial fibrillation or brain cancer.

In yet another aspect, a method is provided for treating a neurological disease, comprising: delivering to a subject a nucleic acid molecule encoding a G protein-coupled receptor or an LGIC, wherein the subject heterologously expresses the G protein-coupled receptor or the LGIC, and administering to the subject a drug that activates the G protein-coupled receptor or the LGIC, thereby treating the neurological disease in the subject, wherein the drug is administered to the subject at least one week after delivery of the nucleic acid molecule encoding the G protein-coupled receptor or the LGIC. In some cases, the nucleic acid molecule encoding the G protein-coupled receptor or the LGIC is delivered to the subject by a viral vector. In some cases, the viral vector is an adenoviral vector, a lentiviral vector or an adeno-associated (AAV) viral vector. In some examples, the AAV vector is AAV-6 or AAV-9. In some examples, the AAV vector is AAV6(Y705+731F+T492V), AAV9(Y731F) or AAV-7m8. In some cases, the nucleic acid molecule is delivered to the subject by a non-viral method. In some instances, the non-viral method is lipofection, nanoparticle delivery, particle bombardment, electroporation, sonication or microinjection. In some cases, the neurological disease is Alzheimer's disease, Parkinson's disease, pain, epilepsy, obesity, anorexia, PTSD, GERD, addiction, anxiety, depression, memory loss, dementia, sleep apnea, stroke, narcolepsy, urinary incontinence, essential tremor, movement disorder, atrial fibrillation or brain cancer. In some cases, the drug is clozapine-N-oxide, nalfurafine (C₂₈H₃₂N₂O₅), salvinorin B, allatostatin, clozapine, olanzapine, perlapine, fluperlapine, alosetron, 8-Chloro-11-[4-(1,1-dideutrioethyl)piperazin-1-yl]-5H-dibenzo[b,e][1,4]diazepine or 11-(Piperazin-1-yl)-5H-dibenzo[b,e][1,4]diazepine. In some cases, the drug is ivermectin, selamectin, doramectin, emamectin, eprinomectin, abamectin, moxidectin, PSEM^{22S}, PSEM^{89S}, PSEM^{9S}, capsaicin, or zolpidem. In some cases, the drug is administered at a dose of 0.001 μg/kg to 10 mg/kg. In some cases, the G protein-coupled receptor or the LGIC is selectively expressed in an excitable cell. In some cases, the excitable cell is a neuron or a myocyte. In some cases, the neuron is a sensory neuron, dorsal root ganglion or trigeminal ganglion. In some cases, the method further comprises administering the drug daily for at least three consecutive days.

In yet another aspect, a method is provided for treating a neurological disease comprising: administering to a subject that heterologously expresses a G protein-coupled receptor or an LGIC a drug that activates the G protein-coupled receptor or the LGIC, wherein the drug is not an endogenous ligand for the G protein-coupled receptor or the LGIC. In some embodiments, the drug is not a kappa-opioid receptor- (KOR) binding drug. In some cases, the neurological disease is not epilepsy. In some cases, the G protein-coupled receptor is a G protein-coupled receptor other than a kappa-opioid receptor (KOR). In some cases, the heterologous G protein-coupled receptor is a DREADD. In some examples, the DREADD is hM4Di, hM3Dq, or AlstR. In some cases, the LGIC is GlyR-M, GluCl, PSAM-5HT3HC, PSAM-GlyR, PSAM-nAChR, TRPV1 or GABAA. In some cases, the drug is clozapine-N-oxide, nalfurafine (C₂₈H₃₂N₂O₅), salvinorin B, allatostatin, clozapine, olanzapine, perlapine, fluperlapine, alosetron, 8-Chloro-11-[4-(1,1-dideutrioethyl)piperazin-1-yl]-5H-dibenzo[b,e][1,4]diazepine or 11-(Piperazin-1-yl)-5H-dibenzo[b,e][1,4]diazepine. In some cases, the drug is ivermectin, selamectin, doramectin, emamectin, eprinomectin, abamectin, moxidectin, PSEM^{22S}, PSEM^{89S}, PSEM^{9S}, capsaicin, or zolpidem. In some cases, the method further comprises, prior to the administering, delivering to the subject a nucleic acid molecule encoding the G protein-coupled receptor or the LGIC. In some cases, the G protein-coupled receptor or the LGIC is delivered by a viral vector. In some cases, the viral vector is an adeno-associated viral (AAV) vector, an adenoviral vector, a lentiviral vector or a Herpes Simplex viral (HSV) vector. In some examples, the AAV vector is AAV-6 or AAV-9. In some examples, the AAV vector is AAV6(Y705+731F+T492V), AAV9(Y731F) or AAV-7m8. In some cases, the nucleic acid molecule is delivered to the subject by a non-viral method. In some cases, the non-viral method is lipofection, nanoparticle delivery, particle bombardment, electroporation, sonication or microinjection. In some cases, the neurological disease is pain. In other cases, the neurological disease is a satiety disorder. In some examples, the satiety disorder is obesity, anorexia nervosa or bulimia nervosa. In other cases, the neurological disease is Alzheimer's disease, Parkinson's disease, pain, epilepsy, obesity, anorexia, PTSD, GERD, addiction, anxiety, depression, memory loss, dementia, sleep apnea, stroke, narcolepsy, urinary incontinence, essential tremor, movement disorder, atrial fibrillation or brain cancer. In some cases, the G protein-coupled receptor or the LGIC is selectively expressed in an excitable cell. In some examples, the excitable cell is a neuron or a myocyte. In some cases, the neuron is a sensory neuron, a dorsal root ganglion, or a trigeminal ganglion. In some cases, the administering comprises oral, intrathecal or topical administration.

In another aspect, a method is provided for treating a neurological disease, comprising administering to a subject that heterologously expresses a G protein-coupled receptor or an LGIC, a drug that activates the G protein-coupled receptor or the LGIC, wherein the drug is not an endogenous ligand for the G protein-coupled receptor or the LGIC and wherein the G protein-coupled receptor or the LGIC is selectively expressed in a sensory neuron, a dorsal root ganglion, a trigeminal ganglion, vagus nerve, brain or a myocyte. In some cases, the G protein-coupled receptor is a DREADD. In some examples, the DREADD is hM4Di, hM3Dq, AlstR or KOR-DREADD. In some cases, the LGIC is GlyR-M, GluCl, PSAM-5HT3HC, PSAM-GlyR, PSAM-nAChR, TRPV1 or GABAA. In some cases, the drug is clozapine-N-oxide, nalfurafine (C₂₈H₃₂N₂O₅), salvinorin B, allatostatin, clozapine, olanzapine, perlapine, fluperlapine, alosetron, 8-Chloro-11-[4-(1,1-dideutrioethyl)piperazin-1-yl]-5H-dibenzo[b,e][1,4]diazepine or 11-(Piperazin-1-yl)-5H-dibenzo[b,e][1,4]diazepine. In some cases, the drug is ivermectin, selamectin, doramectin, emamectin, eprinomectin, abamectin, moxidectin, PSEM^{22S}, PSEM^{89S}, PSEM^{9S}, capsaicin, or zolpidem. In some cases, the method further comprises, prior to said administering, delivering a nucleic acid molecule encoding the G protein-coupled receptor or the LGIC to the subject. In some cases, the nucleic acid molecule is delivered to the subject in a viral vector. In some cases, the viral vector is an adenoviral vector, an adeno-associated viral (AAV) vector, a lentiviral vector, or a Herpes Simplex viral (HSV) vector. In some examples, the AAV vector is derived from AAV-6 or AAV-9. In some examples, the AAV vector is AAV6(Y705+731F+T492V), AAV9(Y731F) or AAV-7m8. In some cases, the nucleic acid molecule is delivered to the subject by a non-viral method. In some cases, the non-viral method is lipofection, nanoparticle delivery, particle bombardment, electroporation, sonication or microinjection. In some cases, the neurological disease is pain. In other cases, the neurological disease is epilepsy. In yet other cases, the neurological disease is a satiety disorder. In some examples, the satiety disorder is obesity, anorexia nervosa or bulimia nervosa. In some cases, the G protein-coupled receptor is Gᵢ- or G_{q}-coupled. In some cases, the administering comprises oral, intrathecal or topical administration. In other cases, the delivering comprises intrathecal, intraganglionic, intracranial, subcutaneous, intraspinal, cisterna magna or intraneural delivery. In some cases, the drug is administered at least one week after the delivering. In some cases, the drug is administered at a dose of 0.001 μg/kg to 10 mg/kg. In some cases, the nucleic acid molecule comprises a synapsin, TRPV1, Naᵥ1.7, Naᵥ1.8, Naᵥ1.9, CamKII, NSE or Advillin promoter. In some cases, the subject is a human.

In yet another aspect, a method is provided for treating a neurological disease in a subject, comprising delivering to the subject a nucleic acid molecule encoding a G protein-coupled receptor or an LGIC, and administering to the subject a drug that activates the G protein-coupled receptor or the LGIC, wherein the drug is FDA-approved, but not FDA-approved for the treatment of the neurological disease. In some cases, the G protein-coupled receptor or the LGIC is expressed in the subject. In some cases, the G protein-coupled receptor is heterologously expressed in the subject. In some cases, the G protein-coupled receptor or the LGIC is homologously expressed in the subject. In some cases, the G protein-coupled receptor or the LGIC is ectopically expressed in the subject. In some cases, the G protein-coupled receptor is a DREADD. In some examples, the DREADD is hM4Di, hM3Dq, AlstR or KOR-DREADD. In some cases, the LGIC is GlyR-M, GluCl, PSAM-5HT3HC, PSAM-GlyR, PSAM-nAChR, TRPV1 or GABAA.

In another aspect, a method is provided for treating a neurological disease, comprising administering to a subject that heterologously expresses a G protein-coupled receptor or the LGIC, a drug that activates the G protein-coupled receptor or the LGIC, wherein the drug is administered at a dose of 0.001 μg/kg to 10 mg/kg. In some cases, the drug is clozapine-N-oxide, nalfurafine (C₂₈H₃₂N₂O₅), salvinorin B, allatostatin, clozapine, olanzapine, perlapine, fluperlapine, alosetron, 8-Chloro-11-[4-(1,1-dideutrioethyl)piperazin-1-yl]-5H-dibenzo[b,e][1,4]diazepine or 11-(Piperazin-1-yl)-5H-dibenzo[b,e][1,4]diazepine. In some cases, the drug is ivermectin, selamectin, doramectin, emamectin, eprinomectin, abamectin, moxidectin, PSEM^{22S}, PSEM^{89S}, PSEM^{9S}, capsaicin, or zolpidem.

In another aspect, a method is provided for treating a neurological disease, comprising delivering to a subject a nucleic acid molecule encoding a G protein-coupled receptor or an LGIC and administering to the subject a drug that activates the G protein-coupled receptor or the LGIC, wherein the drug is administered to the subject daily for at least three consecutive days. In some cases, the drug is administered to the subject at least one week after the delivering.

In yet another aspect, a method is provided for treating a neurological disease, comprising administering to a subject that heterologously expresses a ligand-gated ion channel, a drug that activates the ligand-gated ion channel. In some cases, the drug is not glycine, beta-alanine or taurine. In some aspects, a ligand-gated ion channel comprises an ion conduction pore domain and ligand binding domain created by the fusion of two or more polynucleotide sequences that originally coded for separate polypeptides. In some embodiments, the polynucleotide sequences comprise two or more members of the cys loop receptor gene family. In one embodiment, the ion conduction pore domain conducts anions. In another embodiment, the ion conduction pore domain conducts cations. In some cases, a ligand binding domain is activated by the binding of clozapine-N-oxide, clozapine, perlapine, olanzapine, alosetron, fluperlapine, or N4′-alkyl substituted CNO analogs. In certain aspects, a ligand binding domain is activated by the binding of nicotine, varenicline, or galantamine.

In some cases, the ligand-gated ion channel is GlyR-M, GluCl, PSAM-5HT3HC, PSAM-GlyR, PSAM-nAChR, TRPV1 or GABAA. In some cases, the drug is ivermectin, selamectin, doramectin, emamectin, eprinomectin, abamectin, moxidectin, PSEM^{22S}, PSEM^{89S}, PSEM^{9S}, capsaicin, or zolpidem. In some cases, the method further comprises, prior to the administering, delivering a nucleic acid molecule encoding the ligand-gated ion channel to the subject. In some cases, the nucleic acid molecule is delivered to the subject by a viral vector. In some cases, the viral vector is an adenoviral vector, an adeno-associated viral (AAV) vector, a lentiviral vector, or a Herpes Simplex viral (HSV) vector. In some examples, the AAV vector is derived from AAV-6 or AAV-9. In some examples, the AAV vector is AAV6(Y705+731F+T492V), AAV9(Y731F) or AAV-7m8. In some aspects, the AAV vector comprises SEQ ID NO: 1. In some cases, the nucleic acid molecule is delivered to the subject by a non-viral method. In some cases, the non-viral method is lipofection, nanoparticle delivery, particle bombardment, electroporation, sonication or microinjection. In some cases, the neurological disease is pain. In other cases, the neurological disease is epilepsy. In other cases, the neurological disease is a satiety disorder. In some examples, the satiety disorder is obesity, anorexia nervosa or bulimia nervosa. In some other cases, the neurological disease is Alzheimer's disease, Parkinson's disease, post-traumatic stress disorder (PTSD), gastroesophageal reflux disease (GERD), addiction, anxiety, depression, memory loss, dementia, sleep apnea, stroke, urinary incontinence, narcolepsy, essential tremor, movement disorder, atrial fibrillation or brain cancer. In some cases, the ligand-gated ion channel is selectively expressed in an excitable cell. In some cases, the excitable cell is a neuron or a myocyte. In some cases, the neuron is a dorsal root ganglion, a sensory neuron or a trigeminal ganglion. In some cases, the administering comprises oral, intrathecal or topical administration. In other cases, the delivering comprises intrathecal, intraganglionic, intracranial, subcutaneous, intraspinal, cisterna magna or intraneural delivery. In some cases, the drug is administered at least one week after the delivering. In some cases, the nucleic acid molecule comprises a synapsin, TRPV1, Naᵥ1.7, Naᵥ1.8, Naᵥ1.9, CamKII, NSE or Advillin promoter. In some cases, the subject is a human. In some cases, the subject is a veterinary animal.

In another aspect, a method is provided for treating a neurological disease, comprising administering to a subject that heterologously expresses a ligand-gated ion channel, a drug that activates the ligand-gated ion channel, wherein the drug is administered at a dose of 0.001 μg/kg to 10 mg/kg. In some cases, the ligand-gated ion channel is GlyR-M, GluCl, PSAM-5HT3HC, PSAM-GlyR, PSAM-nAChR, TRPV1 or GABAA. In some cases, the drug is ivermectin, selamectin, doramectin, emamectin, eprinomectin, abamectin, moxidectin, PSEM^{22S}, PSEM^{89S}, PSEM^{9S}, capsaicin, or zolpidem. In some cases, the neurological disease is pain. In some cases, the pain is alleviated.

In various embodiments, the present invention contemplates, in part, an AAV vector comprising a promoter that is operable in a neuronal cell, wherein the promoter is operably linked to a polynucleotide encoding a switch receptor.

In particular embodiments, the promoter is a neuron specific promoter.

In some embodiments, the neuron specific promoter is a promoter operable in a trigeminal ganglion (TGG) neuron or a dorsal root ganglion (DRG) neuron.

In further embodiments, the neuron specific promoter is an hSYN1 promoter, a calcium/calmodulin-dependent protein kinase II a promoter, a tubulin alpha I promoter, a neuron-specific enolase promoter, a platelet-derived growth factor beta chain promoter, TRPV1 promoter, a Nav1.7 promoter, a Nav1.8 promoter, a Nav1.9 promoter, or an Advillin promoter.

In particular embodiments, the neuron specific promoter is an hSYN1 promoter.

In additional embodiments, the promoter is a constitutive promoter.

In particular embodiments, the constitutive promoter is a cytomegalovirus (CMV) immediate early promoter, a viral simian virus 40 (SV40), a Moloney murine leukemia virus (MoMLV) LTR promoter, a Rous sarcoma virus (RSV) LTR, a herpes simplex virus (HSV) (thymidine kinase) promoter, an H5, a P7.5, or a P11 promoter from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 promoter, a Ubiquitin C promoter (UBC), a phosphoglycerate kinase-1 (PGK) promoter, a cytomegalovirus enhancer/chicken β-actin (CAG) promoter, or a β-actin promoter.

In some embodiments, the promoter is an inducible promoter.

In additional embodiments, the inducible promoter is a tetracycline responsive promoter, an ecdysone responsive promoter, a cumate responsive promoter, a glucocorticoid responsive promoter, an estrogen responsive promoter, a PPAR-γ promoter, or an RU-486 responsive promoter.

In additional embodiments, the switch receptor comprises a ligand-gated ion channel or a G-coupled protein receptor.

In particular embodiments, the activity of the switch receptor is regulated by an extracellular ligand.

In particular embodiments, the ligand is non-naturally occurring or synthetic.

In further embodiments, the activity of a cell expressing the switch receptor is increased when an extracellular ligand binds the switch receptor, optionally wherein the activity is electrophysiological activity.

In certain embodiments, the switch receptor is selected from the group consisting of: hM3Dq, GsD, PSAM-5HT3HC, PSAM-nAChR, or TRPV1.

In some embodiments, the ligand is selected from the group consisting of: PSEM22S, PSEM9S, capsaicin, clozapine, perlapine, alosetron, fluperlapine, nalfurafine (C₂₈H₃₂N₂O₅), olanzapine, clozapine-N-oxide, clozapine-N-oxide analogs: 3-chloro-6-(4-ethylpiperazin-1-yl)-5H-benzo[b][1,4]benzodiazepine, 4-(8-Chloro-5H-dibenzo[b,e][1,4]diazepin-11-yl)-1,1-dimethylpiperazin-1-ium iodide, 3-chloro-6-(piperazin-1-yl)-5H-benzo[b][1,4]benzodiazepine, 8-Chloro-11-[4-(1,1-dideutrioethyl)piperazin-1-yl]-5H-dibenzo[b,e][1,4]diazepine, 11-(Piperazin-1-yl)-5H-dibenzo[b,e][1,4]diazepine, and 11-(4-Ethylpiperazin-1-yl)-5H-dibenzo[b,e][1,4]diazepine.

In particular embodiments, the activity of a cell expressing the switch receptor is decreased when the extracellular ligand binds the switch receptor, optionally wherein the activity is electrophysiological activity.

In additional embodiments, the switch receptor is selected from the group consisting of: AlstR, hM4Di, KORD, GluCl, PSAM-GlyR, GlyR-M, and GABA.

In certain embodiments, the switch receptor comprises one or more subunits of a glycine receptor (GlyR) polypeptide.

In some embodiments, the switch receptor comprises a glycine receptor alpha 1 subunit (GlyRα1) polypeptide.

In additional embodiments, the GlyRα1 polypeptide comprises one or more amino acid insertions, deletions, or substitutions.

In particular embodiments, the GlyRα1 polypeptide comprises the amino acid substitutions F207A and A288G. In some embodiments, a switch receptor comprises a GlyRα1 subunit comprising one or more of the following amino acid substitutions: A-1′E, P-2′Δ, T13′V, R19′E, F207A, and A228G (see, Islam et al., ACS Chem. Neurosci., DOI: 10.1021/acschemneuro.6b00168 (2016)). In one embodiment, a switch receptor comprises a GlyRα1 subunit comprising amino acid substitutions A-FE, F207A, and A228G and specifically binds the ligand ivermectin. In another embodiment, a switch receptor comprises a GlyRα1 subunit comprising amino acid substitutions A-FE, P-2′4, T13′V, F207A, and A228G and specifically binds the ligand ivermectin.

In certain embodiments, the ligand is selected from the group consisting of: ivermectin, selamectin, doramectin, emamectin, eprinomectin, abamectin, and moxidectin.

In particular embodiments, the ligand is ivermectin.

In some embodiments, the switch receptor comprises a GluCl α or GluCl β polypeptide.

In additional embodiments, the GluCl α or GluCl β polypeptide comprises one or more amino acid insertions, deletions, or substitutions.

In additional embodiments, the ligand is selected from the group consisting of: ivermectin, selamectin, doramectin, emamectin, eprinomectin, abamectin, and moxidectin.

In particular embodiments, the switch receptor comprises a PSAM-5HT3HC polypeptide.

In particular embodiments, the PSAM-5HT3HC polypeptide comprises one or more amino acid insertions, deletions, or substitutions.

In some embodiments, the ligand is PSEM22S.

In certain embodiments, the switch receptor comprises a PSAM-GlyR polypeptide.

In additional embodiments, the PSAM-GlyR polypeptide comprises one or more amino acid insertions, deletions, or substitutions.

In particular embodiments, the ligand is PSEM89S.

In some embodiments, the switch receptor comprises a PSAM-nAChR polypeptide.

In particular embodiments, the PSAM-nAChR polypeptide comprises one or more amino acid insertions, deletions, or substitutions.

In certain embodiments, the ligand is PSEM9S.

In certain embodiments, the switch receptor comprises a TRPV1 polypeptide.

In additional embodiments, the TRPV1 polypeptide comprises one or more amino acid insertions, deletions, or substitutions.

In further embodiments, the ligand is Capsacin.

In additional embodiments, the switch receptor comprises a GABAA polypeptide.

In further embodiments, the GABAA polypeptide comprises one or more amino acid insertions, deletions, or substitutions.

In particular embodiments, the ligand is Zolpidem.

In additional embodiments, the switch receptor comprises a AlstR polypeptide.

In some embodiments, the AlstR polypeptide comprises one or more amino acid insertions, deletions, or substitutions.

In further embodiments, the ligand is Allatostatin.

In certain embodiments, the switch receptor comprises a hM4Di polypeptide.

In further embodiments, the hM4Di polypeptide comprises one or more amino acid insertions, deletions, or substitutions.

In particular embodiments, the ligand is selected from the group consisting of: CNO, clozapine, perlapine, olanzapine, alosetron, fluperlapine, nalfurafine (C₂₈H₃₂N₂O₅), and N4′-alkyl substituted CNO analogs.

In particular embodiments, the N4′-alkyl substituted CNO analogs are selected from the group consisting of: 3-chloro-6-(4-ethylpiperazin-1-yl)-5H-benzo[b][1,4]benzodiazepine, 4-(8-Chloro-5H-dibenzo[b,e][1,4]diazepin-11-yl)-1,1-dimethylpiperazin-1-ium iodide, 3-chloro-6-(piperazin-1-yl)-5H-benzo[b][1,4]benzodiazepine, 8-Chloro-11-[4-(1,1-dideutrioethyl)piperazin-1-yl]-5H-dibenzo[b,e][1,4]diazepine, 11-(Piperazin-1-yl)-5H-dibenzo[b,e][1,4]diazepine, and 11-(4-Ethylpiperazin-1-yl)-5H-dibenzo[b,e][1,4]diazepine.

In some embodiments, the switch receptor comprises a KORD polypeptide.

In additional embodiments, the KORD polypeptide comprises one or more amino acid insertions, deletions, or substitutions.

In some embodiments, the ligand is Salvinorin B.

In certain embodiments, the switch receptor comprises a hM3Dq polypeptide.

In additional embodiments, the hM3Dq polypeptide comprises one or more amino acid insertions, deletions, or substitutions.

In some embodiments, the ligand is selected from the group consisting of: CNO, clozapine, perlapine, olanzapine, alosetron, fluperlapine, nalfurafine (C₂₈H₃₂N₂O₅), and N4′-alkyl substituted CNO analogs.

In particular embodiments, the N4′-alkyl substituted CNO analogs are selected from the group consisting of: 3-chloro-6-(4-ethylpiperazin-1-yl)-5H-benzo[b][1,4]benzodiazepine, 4-(8-Chloro-5H-dibenzo[b,e][1,4]diazepin-11-yl)-1,1-dimethylpiperazin-1-ium iodide, 3-chloro-6-(piperazin-1-yl)-5H-benzo[b][1,4]benzodiazepine, 8-Chloro-11-[4-(1,1-dideutrioethyl)piperazin-1-yl]-5H-dibenzo[b,e][1,4]diazepine, 11-(Piperazin-1-yl)-5H-dibenzo[b,e][1,4]diazepine, and 11-(4-Ethylpiperazin-1-yl)-5H-dibenzo[b,e][1,4]diazepine.

In certain embodiments, the switch receptor comprises a GsD polypeptide.

In further embodiments, the GsD polypeptide comprises one or more amino acid insertions, deletions, or substitutions.

In some embodiments, the ligand is selected from the group consisting of: CNO, clozapine, perlapine, olanzapine, alosetron, fluperlapine, nalfurafine (C₂₈H₃₂N₂O₅), and N4′-alkyl substituted CNO analogs.

In additional embodiments, the N4′-alkyl substituted CNO analogs are selected from the group consisting of: 3-chloro-6-(4-ethylpiperazin-1-yl)-5H-benzo[b][1,4]benzodiazepine, 4-(8-Chloro-5H-dibenzo[b,e][1,4]diazepin-11-yl)-1,1-dimethylpiperazin-1-ium iodide, 3-chloro-6-(piperazin-1-yl)-5H-benzo[b][1,4]benzodiazepine, 8-Chloro-11-[4-(1,1-dideutrioethyl)piperazin-1-yl]-5H-dibenzo[b,e][1,4]diazepine, 11-(Piperazin-1-yl)-5H-dibenzo[b,e][1,4]diazepine, and 11-(4-Ethylpiperazin-1-yl)-5H-dibenzo[b,e][1,4]diazepine.

In particular embodiments, the vector further comprises a polynucleotide encoding an epitope tag.

In further embodiments, the epitope tag is selected from the group consisting of: maltose binding protein (“MBP”), glutathione S transferase (GST), HIS6, MYC, FLAG, V5, VSV-G, and HA.

In particular embodiments, the vector further comprises a poly(A) sequence.

In additional embodiments, the poly(A) sequence is an SV40 poly(A)sequence, a bovine growth hormone poly(A)sequence (bGHpA), or a rabbit β-globin poly(A)sequence (rβgpA).

In particular embodiments, the poly(A) sequence is a bGHpA.

In certain embodiments, the AAV vector comprises one or more AAV2 inverted terminal repeats (ITRs).

In some embodiments, the AAV vector comprises a serotype selected from the group consisting of: AAV1, AAV1(Y705+731F+T492V), AAV2(Y444+500+730F+T491V), AAV3(Y705+731F), AAV5, AAV5(Y436+693+719F), AAV6, AAV6 (VP3 variant Y705F/Y731F/T492V), AAV-7m8, AAV8, AAV8(Y733F), AAV9, AAV9 (VP3 variant Y731F), AAV10(Y733F), and AAV-ShH10.

In additional embodiments, the AAV vector comprises a serotype selected from the group consisting of: AAV1, AAV5, AAV6, AAV6 (Y705F/Y731F/T492V), AAV8, AAV9, and AAV9 (Y731F).

In certain embodiments, the AAV vector comprises a serotype selected from the group consisting of: AAV6, AAV6 (Y705F/Y731F/T492V), AAV9, and AAV9 (Y731F).

In further embodiments, the AAV vector comprises an AAV6 or AAV6 (Y705F/Y731F/T492V) serotype.

In certain embodiments, the promoter is operable in a DRG neuron or a TGG neuron and the switch receptor comprises a GlyRα1 polypeptide.

In some embodiments, the promoter is a hSYN-1 promoter and the switch receptor comprises a GlyRα1 polypeptide further comprising the amino acid substitutions F207A and A288G.

In particular embodiments, the AAV serotype is AAV1, AAV1(Y705+731F+T492V), AAV2(Y444+500+730F+T491V), AAV3(Y705+731F), AAV5, AAV5(Y436+693+719F), AAV6, AAV6 (VP3 variant Y705F/Y731F/T492V), AAV-7m8, AAV8, AAV8(Y733F), AAV9, AAV9 (VP3 variant Y731F), AAV10(Y733F), or AAV-ShH10, the promoter is a hSYN-1 promoter, and the switch receptor comprises a GlyRα1 polypeptide further comprising the amino acid substitutions F207A and A288G.

In various embodiments, the present invention contemplates, in part, an AAV vector comprising one or more AAV2 ITRs, an AAV6 serotype, a hSYN-1 promoter, and a polynucleotide encoding a GlyRα1 polypeptide further comprising the amino acid substitutions F207A and A288G.

In various embodiments, the present invention contemplates, in part, an AAV vector comprising one or more AAV2 ITRs, an AAV6 (Y705F/Y731F/T492V) serotype, a hSYN-1 promoter, and a polynucleotide encoding a GlyRα1 polypeptide further comprising the amino acid substitutions F207A and A288G. In some aspects, the AAV vector comprises SEQ ID NO: 1.

In some embodiments, the AAV vector further comprises a bGHpA.

In certain embodiments, the AAV vector further comprises a FLAG epitope tag.

In certain embodiments, the AAV vector is a self-complementary AAV (scAAV) vector.

In various embodiments, the present invention contemplates, in part, a composition comprising one or more of the vectors described herein.

In various embodiments, the present invention contemplates, in part, a method of managing, preventing, or treating pain in a subject, comprising administering to the subject an AAV vector described herein.

In various embodiments, the present invention contemplates, in part, a method of providing analgesia to a subject having pain, comprising administering to the subject an AAV vector described herein.

In further embodiments, the pain is acute pain or chronic pain.

In some embodiments, the pain is chronic pain.

In particular embodiments, the pain is acute pain, chronic pain, neuropathic pain, nociceptive pain, allodynia, inflammatory pain, inflammatory hyperalgesia, neuropathies, neuralgia, diabetic neuropathy, human immunodeficiency virus-related neuropathy, nerve injury, rheumatoid arthritic pain, osteoarthritic pain, burns, back pain, eye pain, visceral pain, cancer pain (e.g., bone cancer pain), dental pain, headache, migraine, carpal tunnel syndrome, fibromyalgia, neuritis, sciatica, pelvic hypersensitivity, pelvic pain, post herpetic neuralgia, post-operative pain, post stroke pain, or menstrual pain.

In additional embodiments, the pain is nociceptive pain.

In certain embodiments, the pain is nociceptive pain is selected from the group consisting of central nervous system trauma, strains/sprains, burns, myocardial infarction and acute pancreatitis, post-operative pain (pain following any type of surgical procedure), posttraumatic pain, renal colic, cancer pain and back pain.

In some embodiments, the pain is neuropathic pain.

In additional embodiments, the etiology of the neuropathic pain is selected from the group consisting of: peripheral neuropathy, diabetic neuropathy, post herpetic neuralgia, trigeminal neuralgia, back pain, cancer neuropathy, HIV neuropathy, phantom limb pain, carpal tunnel syndrome, central post-stroke pain and pain associated with chronic alcoholism, hypothyroidism, uremia, multiple sclerosis, spinal cord injury, Parkinson's disease, epilepsy, and vitamin deficiency.

In particular embodiments, the neuropathic pain is related to a pain disorder selected from the group consisting of: arthritis, allodynia, a typical trigeminal neuralgia, trigeminal neuralgia, somatoform disorder, hypoesthesis, hypealgesia, neuralgia, neuritis, neurogenic pain, analgesia, anesthesia dolorosa, causlagia, sciatic nerve pain disorder, degenerative joint disorder, fibromyalgia, visceral disease, chronic pain disorders, migraine/headache pain, chronic fatigue syndrome, complex regional pain syndrome, neurodystrophy, plantar fasciitis or pain associated with cancer.

In further embodiments, the pain is inflammatory pain.

In certain embodiments, the pain is associated with musculoskeletal disorders, myalgia, fibromyalgia, spondylitis, sero-negative (non-rheumatoid) arthropathies, non-articular rheumatism, dystrophinopathy, glycogenolysis, polymyositis and pyomyositis; heart and vascular pain, pain caused by angina, myocardical infarction, mitral stenosis, pericarditis, Raynaud's phenomenon, scleredoma and skeletal muscle ischemia; head pain, migraine, cluster headache, tension-type headache mixed headache and headache associated with vascular disorders; orofacial pain, dental pain, otic pain, burning mouth syndrome, and temporomandibular myofascial pain.

In particular embodiments, a method comprises intrathecal administration of an AAV vector or composition contemplated herein.

In particular embodiments, a method comprises intraganglionic administration of an AAV vector or composition contemplated herein.

In particular embodiments, a method comprises intraneural administration of an AAV vector or composition contemplated herein.

### BRIEF DESCRIPTION OF THE DRAWINGS

FIG. 1 depicts a method of the disclosure utilizing the compositions as disclosed herein. FIG. 1 depicts genetic insertion of a therapeutic ‘switch’ receptor of the disclosure (e.g., a G protein coupled receptor or ligand-gated ion channel) into the dorsal root ganglion associated with a damaged peripheral nerve via a viral vector and activation of the ‘switch’ with a biologically inert compound to silence neural communication with the central nervous system, thereby effectively providing analgesia and blocking painful sensations.

FIG. 2A-FIG. 2C depict non-limiting examples of AAV transfer vector architectures for delivery of switch receptors of the disclosure including (FIG. 2A) the human synapsin (hSYN) promoter driving expression of hM4Di, (FIG. 2B) hSYN-hM3Dq, and (FIG. 2C) hSYN-hGlyR(F207A/A288G).

FIG. 3 depicts non-limiting examples of FDA-approved drugs that can be repurposed to treat neurological diseases using the methods and compositions described herein.

FIG. 4 depicts a diagram of an exemplary gene therapy vector contemplated herein.

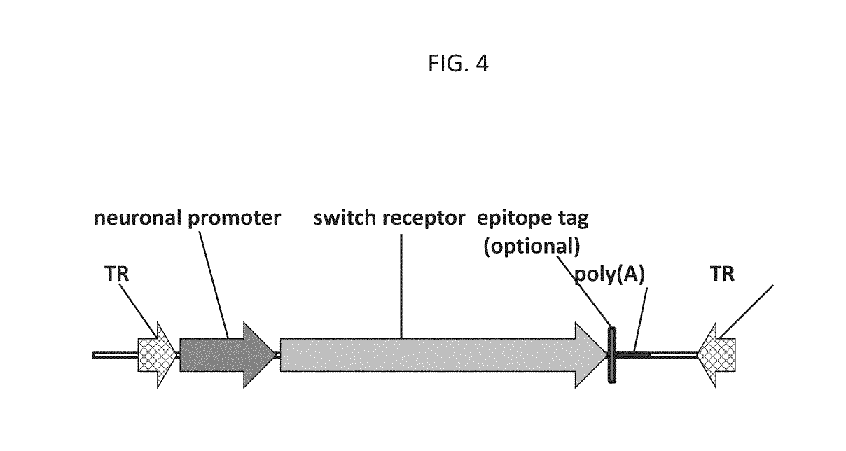

FIG. 5 depicts a diagram of a hSYN1-GlyRα1 F207A/A288G gene therapy vector.

FIG. 6 depicts a diagram of pain neurons and pathways in the spinal cord and skin and deep tissues. FIG. 6 also shows results of immunohistochemistry analysis of the expression of the AAV6(Y705+731F+T492V) vector expressing hSYN-hGlyRM(F207A/A288G) in dorsal horn and dorsal root ganglion neurons following intraspinal, intraganglionic, and intrathecal routes of administration at three weeks post injection in mice.

FIG. 7 depicts a diagram of the spared nerve injury (SNI) model in mice. FIG. 7 also depicts a graph showing the results of a mechanical hypersensitivity assay (Von Frey) in a mouse SNI model injected with SWB001 (AAV6 vector expressing hSYN-hGlyRM(F207A/A288G)) compared with an uninjured contralateral control. A single dose of ivermectin (15 mg/kg) was injected IP following nerve injury to provide analgesia for 7-10 days following nerve injury.

FIG. 8 depicts a graph showing the results of a mechanical hypersensitivity assay (Von Frey) in a mouse SNI model injected with SWB001 (AAV6 vector expressing hSYN-hGlyRM(F207A/A288G)) compared with an uninjured contralateral control. After pain threshold returned to baseline level following washout of ivermectin in the experiment described in FIG. 7, a repeat dose of ivermectin (10 mg/kg) was injected IP to provide analgesia at 14 days post nerve injury.

FIG. 9 depicts a graph showing individual subject results of a thermal withdrawal latency assay described in Example 22. For each subject, the pre-ivermectin treatment result is shown on the left, and the post-ivermectin treatment result is shown on the right.

FIG. 10 depicts a graph showing average subject results of a thermal withdrawal latency assay described in Example 22. The pre-ivermectin treatment result is shown on the left, and the post-ivermectin treatment result is shown on the right.

### DETAILED DESCRIPTION OF THE INVENTION

### A. Overview

The present disclosure provides compositions and methods for treating neurological diseases and disorders. The compositions generally include therapeutic receptors referred to herein as “switch receptors.” In some examples, a switch receptor is a G protein-coupled receptor (GPCR) or a ligand-gated ion channel (LGIC). The compositions and methods herein may find particular use as e.g., gene therapy for the treatment of neurological disease. In some cases, the methods provide for administering a composition to a subject in need thereof. A subject in need thereof can be a subject suffering from a neurological disease. In some cases, the switch receptor is expressed in the subject suffering from a neurological disease. The methods further provide for treating the subject with a ligand that activates the expressed switch receptor. Treatment with the ligand may alter the electrophysiological activity of e.g., an excitable cell (e.g., neuron, muscle cell) expressing the switch receptor, thereby treating the neurological disease.

In some embodiments, the invention generally relates to gene therapies for the management of pain. The gene therapy compositions and methods contemplated herein offer precise spatiotemporal control over neuronal cells involved in the pain pathway and thus, also offer numerous advantages compared to existing therapies. Without wishing to be bound by any particular theory, it is contemplated that delivering gene therapies targeting neuronal cells can mediate pain relief over an extended duration, reduce side effects, and improve quality of life by freeing patients from external pumps and hazardous procedures. Moreover, gene therapy also offers numerous payload advantages compared to conventional drug equivalents, such as, for example, certain larger proteins may not be available as a recombinant product or a small molecule analog, but can be encoded and delivered as a therapeutic gene in a vector.

In various embodiments, a viral vector comprising one or more expression control sequences capable of expressing a transcript in a neuronal cell operably linked to a polynucleotide encoding a switch receptor is provided. The present invention contemplates that the switch receptors that bind exogenously supplied and/or non-naturally occurring ligands can be delivered to neuronal cells using viral vectors and can be used to modulate the activity, e.g., electrophysiological activity, of the neuronal cells to safely and efficiently manage pain in a subject.

In particular embodiments, parvoviral vectors including adeno-associated virus (AAV) vectors comprising expression control elements active in neuronal cells operably linked to a switch receptor that comprises a ligand-gated ion channel, g-protein coupled receptor (GPCR), or subunits and/or muteins thereof is provided.

The vectors and compositions contemplated herein are used to attenuate the sensation of pain in a subject. In various embodiments, the pain is acute pain or chronic pain. The chronic pain can be nociceptive pain or neuropathic pain. In one embodiment, the pain is neuropathic pain. The pain can also be an isolated pain, or the pain can be associated with a particular disease.

Accordingly, the present invention addresses an unmet clinical need for improving the safety and efficacy of gene therapy in pain management.

The practice of the present invention will employ, unless indicated specifically to the contrary, conventional methods of molecular biology and recombinant DNA techniques within the skill of the art, many of which are described below for the purpose of illustration. Such techniques are explained fully in the literature. See, e.g., Sambrook, et al., Molecular Cloning: A Laboratory Manual (2nd Edition, 1989); Maniatis et al., Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); A Practical Guide to Molecular Cloning (B. Perbal, ed., 1984); Harlow and Lane, eds. (1988) Antibodies, A Laboratory Manual; the series Methods In Enzymology (Academic Press, Inc.); PCR 2: A Practical Approach (M. J. MacPherson, B. D. Hames and G. R. Taylor eds. (1995)).

All publications, patents and patent applications cited herein are hereby incorporated by reference in their entirety.

### B. Definitions

Unless defined otherwise, all technical and scientific terms used herein have the same meaning as commonly understood by those of ordinary skill in the art to which the invention belongs. For the purposes of the present invention, the following terms are defined below.

The articles “a,” “an,” and “the” are used herein to refer to one or to more than one (i.e. to at least one) of the grammatical object of the article. By way of example, “an element” means one element or more than one element.

The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

Throughout this specification, unless the context requires otherwise, the words “comprise”, “comprises” and “comprising” will be understood to imply the inclusion of a stated step or element or group of steps or elements but not the exclusion of any other step or element or group of steps or elements. In particular embodiments, the terms “include,” “has,” “contains,” and “comprise” are used synonymously.

By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “isolated” means material that is substantially or essentially free from components that normally accompany it in its native state. In particular embodiments, the term “obtained” or “derived” is used synonymously with isolated.

The terms “subject,” “patient” and “individual” are used interchangeably herein to refer to a vertebrate, preferably a mammal, more preferably a human. Mammals include, but are not limited to, murines, simians, humans, farm animals, sport animals, and pets. Tissues, cells, and their progeny of a biological entity obtained in vivo or cultured in vitro are also encompassed. A “subject,” “patient” or “individual” as used herein, includes any animal that exhibits pain that can be treated with the vectors, compositions, and methods contemplated herein. Suitable subjects (e.g., patients) include laboratory animals (such as mouse, rat, rabbit, or guinea pig), farm animals, and domestic animals or pets (such as a cat or dog). Non-human primates and, preferably, human patients, are included.

As used herein “treatment” or “treating,” includes any beneficial or desirable effect associated with a reduction in pain, and may include even minimal reductions in pain. Treatment can involve optionally either the reduction or amelioration of pain, or the delaying of the progression of pain. “Treatment” does not necessarily indicate complete eradication or cure of the disease or condition, or associated symptoms thereof.

As used herein, “prevent,” and similar words such as “prevented,” “preventing” etc., indicate an approach for preventing, inhibiting, or reducing the likelihood of the occurrence or recurrence of pain. It also refers to delaying the onset or recurrence of a disease or condition or delaying the occurrence or recurrence of the symptoms of pain. As used herein, “prevention” and similar words also includes reducing the intensity, effect, symptoms and/or burden of pain prior to onset or recurrence.

As used herein, “management” or “controlling” pain refers to the use of the compositions or methods contemplated herein, to improve the quality of life for an individual by provide analgesia to a subject suffering from pain.

As used herein, the term “amount” refers to “an amount effective” or “an effective amount” of a virus to achieve a beneficial or desired prophylactic or therapeutic result, including clinical results.

A “prophylactically effective amount” refers to an amount of a virus effective to achieve the desired prophylactic result. Typically but not necessarily, since a prophylactic dose is used in subjects prior to or at an earlier stage of disease, the prophylactically effective amount is less than the therapeutically effective amount.

A “therapeutically effective amount” of a virus may vary according to factors such as the disease state, age, sex, and weight of the individual, and the ability of the stem and progenitor cells to elicit a desired response in the individual. A therapeutically effective amount is also one in which any toxic or detrimental effects of the virus are outweighed by the therapeutically beneficial effects. The term “therapeutically effective amount” includes an amount that is effective to “treat” a subject (e.g., a patient).

An “increased” or “enhanced” amount of a physiological response, e.g., electrophysiological activity or cellular activity, is typically a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the level of activity in an untreated cell.

A “decrease” or “reduced” amount of a physiological response, e.g., electrophysiological activity or cellular activity, is typically a “statistically significant” amount, and may include an decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the level of activity in an untreated cell.

By “maintain,” or “preserve,” or “maintenance,” or “no change,” or “no substantial change,” or “no substantial decrease” refers generally to a physiological response that is comparable to a response caused by either vehicle, or a control molecule/composition. A comparable response is one that is not significantly different or measurable different from the reference response.

As used herein, the term “excitable cell” refers to a cell that experiences fluctuations in its membrane potential as a result of gated ion channels. Illustrative examples of excitable cells contemplated herein include but are not limited to myocytes, neuronal cells, and the like.

In particular embodiments, the neuronal cell is a sensory neuron. Illustrative examples of sensory neurons include, but are not limited to, dorsal root ganglion (DRG) neurons and trigeminal ganglion (TGG) neurons. In one embodiment, the neuronal cell is a peripheral sensory neuron. In one embodiment, the neuronal cell is an inhibitory interneuron.

“G protein-coupled receptor” or “GPCR” means a receptor that, upon binding of its natural peptide or nonpeptide ligand and activation of the receptor, transduces a G protein-mediated signal(s) that results in a physiological, cellular response (e.g., cell proliferation or secretion). G protein-coupled receptors form a large family of evolutionarily related proteins. Proteins that are members of the G protein-coupled receptor family are generally composed of seven putative transmembrane domains. G protein-coupled receptors are also known in the art as “seven transmembrane segment (7TM) receptors” and as “heptahelical receptors.”

“Ligand-gated ion channel” refers to a large group of intrinsic transmembrane proteins that allow passage of ions upon activation by a specific chemical. Most endogenous ligands bind to a site distinct from the ion conduction pore and binding directly causes opening or closing of the channel. Endogenous ligands can bind extracellularly, e.g., glutamate, ACh and GABA, or intracellularly, e.g. Ca²⁺ on Ca²⁺-activated potassium channels. It is important to note that the ligand itself is not transported across the membrane. Ligand binding causes a drastic change in the permeability of the channel to a specific ion or ions; effectively no ions can pass through the channel when it is inactive but up to 10⁷ ions per second can be allowed through upon ligand binding.

“Receptor-ligand binding,” “ligand binding,” and “binding” are used interchangeably herein to mean physical interaction between a receptor (e.g., a G protein-coupled receptor) and a ligand (e.g., a natural ligand, (e.g., peptide ligand) or synthetic ligand (e.g., synthetic small molecule ligand)). Ligand binding can be measured by a variety of methods known in the art (e.g., detection of association with a radioactively labeled ligand).

“Binding affinity” generally refers to the strength of the sum total of noncovalent interactions between a single binding site of a receptor and a ligand. Unless indicated otherwise, as used herein, “binding affinity” refers to intrinsic binding affinity which reflects a 1:1 interaction between members of a binding pair (e.g., receptor and ligand). The affinity of a molecule X for its partner Y can generally be represented by the dissociation constant (Kd). Affinity can be measured by common methods known in the art, including those described herein.

As used herein, the terms “specific binding affinity” or “specifically binds” or “specifically bound” or “specific binding” are used interchangeably throughout the specification and claims and refer to that binding which occurs between a paired species of molecules, e.g., receptor and ligand. When the interaction of the two species produces a non-covalently bound complex, the binding which occurs is typically electrostatic, hydrogen-bonding, or the result of lipophilic interactions. In various embodiments, the specific binding between one or more species is direct. In one embodiment, the affinity of specific binding is about 2 times greater than background binding (non-specific binding), about 5 times greater than background binding, about 10 times greater than background binding, about 20 times greater than background binding, about 50 times greater than background binding, about 100 times greater than background binding, or about 1000 times greater than background binding or more.

“Signaling” refers to the generation of a biochemical or physiological response as a result of ligand binding (e.g., as a result of ligand binding to a switch receptor).

The term “to trigger” refers to the opening of a receptor following physical or chemical stimulation to allow ions to pass passively through the receptor from a region of higher ion concentration to a region of lower concentration.

In general, “sequence identity” or “sequence homology” refers to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby, and comparing these sequences to a second nucleotide or amino acid sequence. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA 87:2264-2268 (1990) and as discussed in Altschul, et al., J. Mol. Biol. 215:403-410 (1990); Karlin And Altschul, Proc. Natl. Acad. Sci. USA 90:5873-5877 (1993); and Altschul et al., Nucleic Acids Res. 25:3389-3402 (1997). Briefly, the BLAST program defines identity as the number of identical aligned symbols (generally nucleotides or amino acids), divided by the total number of symbols in the shorter of the two sequences. The program may be used to determine percent identity over the entire length of the proteins being compared. Default parameters are provided to optimize searches with short query sequences in, for example, with the blastp program. The program also allows use of an SEG filter to mask-off segments of the query sequences as determined by the SEG program of Wootton and Federhen, Computers and Chemistry 17:149-163 (1993). Ranges of desired degrees of sequence identity are approximately 80% to 100% and integer values therebetween. Typically, the percent identities between a disclosed sequence and a claimed sequence are at least 80%, at least 85%, at least 90%, at least 95%, or at least 98%.

The term “biopharmaceutical” as used herein refers to any composition that includes a biologic or biologic medical product that can be utilized as a medicine or therapeutic. The biologic can be any biologic that can be used as a therapeutic agent. A biologic can be any medicinal agent that is manufactured in, extracted from, or semi-synthesized from a biological source. Biologics can include, without limitation, proteins, nucleic acid molecules, cells, tissues, vaccines, blood or blood components, allergenics, gene therapies, recombinant proteins and recombinant nucleic acid molecules. A biopharmaceutical may include additional agents including, without limitation, additional therapies (biologic or synthetic chemical agents), excipients, and the like.

The term “exogenous” is used herein to refer to any molecule, including nucleic acids, protein or peptides, small molecular compounds, and the like that originate from outside the organism. In contrast, the term “endogenous” refers to any molecule that originates from inside the organism (i.e., naturally produced by the organism).

The terms “heterologous expression” and “heterologously expressed” are used herein to refer to the expression of a protein in a subject that ordinarily does not express that protein. Heterologous expression can also refer to the expression of a protein in a subject wherein the protein is derived from a species other than the subject in which the protein is expressed. Heterologous expression may involve the delivery of an exogenous nucleic acid molecule to a subject by any means known to those of skill in the art, including viral vector delivery, electroporation, infection, transfection and the like.

The terms “homologous expression” and “homologously expressed” are used herein to refer to the overexpression of a protein in a subject that ordinarily expresses that protein. Homologous expression may encompass “ectopic expression” which is used herein to refer to the homologous expression of a protein, wherein the protein is expressed in a host cell of the subject that ordinarily does not express that protein (e.g., a protein only found in a myocardial cell of a subject is expressed in a brain cell of the subject).

The term “wild-type” is used herein to refer to a molecule, typically a protein, that is identical or substantially identical to a protein found in nature. The identity of the protein is generally measured as the percentage of sequence identity or homology to a protein ordinarily found in nature. Thus, a wild-type protein can be envisioned as a protein that shares a sequence homology of at least 95%, 96%, 97%, 98%, 99%. 99.5%, 99.9%, or 100% with a protein ordinarily found in nature.

### C. Compositions

In some aspects, the disclosure herein provides for compositions for the treatment of neurological diseases or disorders. The compositions envisioned herein generally include a therapeutic agent that can be used for the treatment of neurological diseases. A therapeutic agent of the disclosure can be any molecule (e.g., protein, RNA, DNA) that is delivered to a subject. In some cases, the subject is a patient suffering from a neurological disease or a condition. The therapeutic agent can be used to treat a neurological disease or can be used to alleviate the symptoms of neurological disease. In some cases, the subject is healthy and the therapeutic agent is used as a prophylactic treatment to prevent the onset of a neurological disease. Generally, the therapeutic agent is delivered to a subject in order to elicit a therapeutic response in the subject. In some embodiments, the compositions are used to treat pain.

### D. Switch Receptors

In various illustrative embodiments, the present invention contemplates, in part, polynucleotides encoding switch receptors and vectors comprising the same and use of these compositions to regulate the activity of a neuronal cell. As used herein, the term “switch receptor” refers to a G protein-coupled receptor (GPCR), a receptor activated solely by synthetic ligand (RASSLs), a designer receptor exclusively activated by designer drug (DREADDs), and/or a ligand-gated ion channel (LGIC) and/or muteins thereof. In some aspects, one or more of the subunits of a switch receptor have been engineered to specifically bind to a heterologous ligand, an exogenous ligand and/or a synthetic ligand. In particular embodiments, “switch receptor” refers to one or more subunits of a GPCR, RASSL, DREADD, or LGIC or mutein thereof engineered to specifically bind to a heterologous, an exogenous and/or a synthetic ligand. The switch receptors contemplated herein are designed to activate, inhibit, depolarize, and/or hyperpolarize neuronal cells.

In particular embodiments, the switch receptor is designed to specifically bind to a heterologous, an exogenous and/or a synthetic ligand that does not detectably bind to a naturally occurring receptor. In certain embodiments, the heterologous, exogenous and/or synthetic ligand specifically binds a switch receptor to regulate the activity of an excitable cell expressing the switch receptor and detectably binds a naturally occurring receptor, but does not elicit a physiologically measurable change upon binding the naturally occurring receptor.

In particular embodiments, a switch receptor is introduced into a neuronal cell using a vector contemplated herein. In particular cases, a nucleic acid molecule encoding a switch receptor is delivered to a subject such that the switch receptor is expressed in at least one host cell. In some cases, a switch receptor is delivered directly to a subject (i.e., as a protein). The switch receptor may be from the same species as the neuronal cell or from a different species. Switch receptors can be heterologously expressed or homologously expressed in a subject. In particular embodiments, the switch receptor comprises one or more amino acid insertions, deletions, or substitutions to allow the switch receptor to be triggered by a heterologous and/or synthetic ligand and to decrease, reduce, or abolish sensitivity to an endogenous ligand. In various embodiments, a switch receptor selected for use would (i) carry a current of suitable polarity and/or ionic composition and (ii) be gated directly by a ligand that is (iii) not used as a neurotransmitter in the nervous system (particularly where the cell to be activated is a neuronal cell).

In one embodiment, a switch receptor is engineered to specifically bind to a heterologous, an exogenous and/or a synthetic ligand that does not detectably bind to a naturally occurring receptor. The atomic structure of the extracellular ligand binding domain of the switch receptor may be determined or predicted using methods known in the art. A high-resolution structure can be used to guide the “rational design” of mutations in the receptor's ligand-binding domain that abolish sensitivity to endogenous ligand. “Second-site” substitutions may then be made on the endogenous ligand to complement these mutations in the receptor and restore functional (but entirely unnatural) receptor-ligand pairs. In another embodiment, docking algorithms can be used to model binding of synthetic ligands to mutated receptor's ligand binding domain. In a particular embodiment, less targeted, or even random mutations could also be used to create mutant species of a switch receptor lacking affinity for a natural agonist. Libraries of potential agonist compounds can then be screened to identify useful non-natural agonists using known “directed evolution” methods.

Similar chemical genetic approaches are also useful for altering the conducting properties of the switch receptor, such as ion selectivity. Chemical genetic approaches may be used to alter the ligand binding, physical activation properties, or conducting properties of a switch receptor. In one non-limiting example, a switch receptor can be engineered to increase the efflux of potassium ions or to increase the influx of anions, such as chloride ions, instead of increasing influx of sodium or calcium ions. When such switch receptors are expressed in neuronal cells and triggered by ligand binding, the engineered receptor would hyperpolarize and inactivate the neuronal cells.

A “heterologous” ligand refers to a polypeptide or small molecule that is from a different species than the species of cell that expresses a switch receptor. A heterologous ligand may be isolated from a natural source, recombinantly produced, or synthetic.

A “naturally occurring ligand” refers to a biomolecule that can be found in nature, which biomolecule binds to a native GPCR or ligand-gated ion channel.

A “synthetic ligand” refers to a polypeptide or small molecule that does not occur in nature and that is synthesized by natural or chemical means. A synthetic ligand may be unique or known.

A “small molecule” refers to a compound that has a molecular weight of less than about 5 kD, less than about 4 kD, less than about 3 kD, less than about 2 kD, less than about 1 kD, or less than about 0.5kD. Small molecules can be nucleic acids, peptides, polypeptides, peptidomimetics, peptoids, carbohydrates, lipids or other organic or inorganic molecules.

In particular embodiments, switch receptors contemplated herein can also be designed to provide variable temporal control, e.g., by varying the onset and offset kinetics (on the order of milliseconds, seconds, minutes, or hours) and to provide variable spatial resolution by using different viral vectors and/or altering the method of delivery and/or delivery site.

1. G Protein-Coupled Receptor (GPCR)

G protein-coupled receptors (GPCRs) are a diverse family of protein receptors that mediate cellular responses to outside stimuli. In particular embodiments, the switch receptor is a GPCR or mutein thereof, a RASSL, or a DREADD. In some aspects, one or more of the subunits of a GPCR or mutein thereof, a RASSL, or a DREADD have been engineered to specifically bind to a heterologous ligand, an exogenous ligand and/or a synthetic ligand. Illustrative examples of GPCRs that specifically bind a ligand, RASSLs, and DREADDs that are suitable for use in particular embodiments and methods for identifying and making the same have been described in Conklin et al., 2008; Pei et al., 2008; Nichols and Roth, 2009; and Dong et al., 2010a, and reviewed in Rogan and Roth, 2011, each of which is incorporated by reference herein, in its entirety.

The compositions of the disclosure can include a nucleic acid molecule encoding the GPCR. In some cases, the GPCR is heterologously expressed in a subject. In other cases, the GPCR is homologously expressed in a subject. In particular cases, the GPCR is ectopically expressed (e.g., in a neuron). The GPCR can include a wild-type GPCR or a mutant GPCR. GPCRs may be derived from essentially any organism in which GPCRs are normally expressed including, without limitation: mammals including humans, mice, rats; insects including Drosophila melanogaster; nematodes including Caenorhabditis elegans; and yeast. The GPCR may be, using the methods described herein, expressed in a subject to treat a neurological disease. In some examples, the GPCR expressed in a subject is derived from a species other than that of the subject. For example, a GPCR ordinarily expressed in a mouse could be expressed in a human using the methods disclosed herein. The GPCRs used in the compositions will be substantially homologous to (i.e., share sequence identity with) a wild-type GPCR, for example, at least 85%, 86%, 87%, 88%, 89%, 90%, 91%, 92%, 93%, 94%, 95%, 96%, 97%, 98%, 99%, 99.5%, 99.9% or 100% identical to a wild-type GPCR.

GPCRs can be classified according to the signaling proteins they interact with. Without wishing to be bound by theory, GPCRs couple to downstream signaling molecules (e.g., G proteins) to transduce an extracellular signal. G proteins can be excitatory (e.g., Gₛ, G_{q/11}, G_{12/13}) or inhibitory (e.g., G_{i/o}). In some cases, activation of a GPCR coupled to a downstream G protein may alter the electrophysiological activity of an excitable cell (e.g., a muscle cell, a neuron). GPCRs may be selected based on the class of G protein to which they couple. It will be clear to one of skill in the art that a GPCR will be selected to perform the methods of the disclosure based on the desired downstream signaling pathways. In one particular example, an inhibitory GPCR (i.e., coupled to G_{i/o}) may be selected to treat pain.

In some cases, GPCRs can be constitutively active (i.e., continuously active in a cell). In this example, the GPCR may not require activation by a ligand. In more particular cases, the GPCR is activated by a ligand. The ligand can be an endogenous or an exogenous ligand. In some examples, the GPCR is activated by an endogenous ligand (i.e., a ligand that is naturally produced by the subject). In other examples, the GPCR is activated by an exogenous ligand (i.e., a ligand that is delivered to the subject by e.g., injection). Ligands can be any molecule that activates the GPCR, including proteins, lipids, synthetic molecules, nucleic acids, and the like. In particular cases, a ligand is delivered to a subject heterologously expressing a GPCR to treat a neurological disease. In one example, the GPCR is allatostatin receptor (AlstR) derived from Drosophila melanogaster and the ligand is allatostatin.

Non-limiting examples of GPCRs suitable for use as described herein include: CHRM1; GNRHR; GPR73; GPR45; PTHR1; CHRM2; GNRHR2; GPR73; GPR63; PTHR2; CHRM3; HRH1; GPR10; GPR83; SCTR; CHRM4; HRH2; F2R; PGR15; ADCYAP1R1; CHRM5; HRH3; F2RL1; PGR15L; VIPR1; ADORA1; HRH4; F2RL2; GPR103; VIPR2; ADORA2A; FSHR 93; F2RL3; GPR103L; BAI1; ADORA2B; LHCGR; P2RY1; GRCA; BAI2; ADORA3; TSHR; P2RY2; PGR1; BAI3; P2RY12; GPR54; P2RY4; HGPCR11; CD97; GPR105; LTB4R; P2RY6; SALPR; EMR1; GPR86; LTB4R2; P2RY11; MAS1; EMR2; GPR87; MRGX1; LGR7; GPR90; EMR3; ADRA1A; MRGX2; LGR8; P2Y5; PGR16; ADRA1B; MRGX3; RGR; GPR23; LEC1; ADRA1D; MRGX4; HTR1A; P2Y10 275; LEC2; ADRA2A; MRGD; HTR1B; FKSG79; LEC3; ADRA2B; MrgA1; HTR1D; PGR2; CELSR1; ADRA2C; MrgA2; HTR1E; PGR3; CELSR2; ADRB1; MrgA3; HTR1F; AGR9; CELSR3; ADRB2 21; MrgA4; HTR2A; CMKLR1; GPR64; ADRB3; MrgA5; HTR2B; EBI2; PGR17; ADMR; MrgA6; HTR2C; GPCR150; DJ287G14; C3AR1; MrgA7; HTR4; GPR1; KIAA0758; C5R1; MrgA8; HTR5A; GPR15; PGR18; GPR77; MrgA9; HTR5B; GPR17; PGR19; AGTR1; MrgA10; HTR6; GPR18; PGR20; AGTR2; MrgA11; HTR7; GPR19; TEM5; AGTRL1; MrgA12; SSTR1; GPR20; KIAA1828; BRS3; MrgA13; SSTR2; GPR22; PGR21; GRPR 31; MrgA14; SSTR3; GPR25; ETL; NMBR; MrgA15; SSTR4; GPR30; FLJ14454; BDKRB1; MrgA16; SSTR5; GPR31; GPR56; BDKRB2; MrgA19; G2A; GPR32; OA1; CNR1; MrgB1; GPR4; GPR33; PGR22;CNR2; MrgB2; GPR65; GPR34; PGR23; CCR1; MrgB3; GPR68; GPR35; PGR24; CCR2; MrgB4; EDG1; GPR39; PGR25; CCR3; MrgB5; EDG2; GPR40; PGR26; CCR4; MrgB6; EDG3; GPR44; PGR27; CCR5 41; MrgB8; EDG4; GPR55; VLGR1; CCR6; MrgB10; EDG5; GPR61; CCR7 43; MrgB11; EDGE; GPR62; CCR8; MrgB13; EDG7; GPR75; CCR9; GPR24; EDG8; GPR80; GPR2; SLT; TACR1; GPR82; CASR; CCRL1; MC1R; TACR2; GPR84; GABBR1; CCRL2; MC2R; TACR3; GPR88; GPR51; CCBP2; MC3R; TRHR; GPR91; GPRC5B; CMKBR1L1; MC4R; TRHR2; GPR92; GPRC5C; CMKBR1L2; MC5R; GPR57; GPR101; GPRC5D; CCXCR1; MTNR1A; GPR58; H963; RAI3; CX3CR1; MTNR1B; PNR; HGPCR2; GRM1; IL8RA; GPR50; TAR1; HGPCR19; GRM2; IL8RB; GPR66; TAR2; HUMNPIIY20; GRM3; GPR9; NMU2R; TAR3; MRG; GRM4; CXCR4; NPFF1R; TAR4; MRGE; GRM5; BLR1; GPR74; GPR102; MRGF; GRM6; CXCR6; GPR7; TA7; MRGG; GRM7; CCKAR; GPR8; TAB; OPN3; GRM8; CCKBR; NPY1R; TA10; OPN4; GPRC6A; CYSLT1; NPY2R; TA11; PGR4; PGR28; CYSLT2; PPYR1; TA12; PGR5; DRD1; NPY5R; TA14; PGR6; DRD2; NPY6R; TA15; PGR7; DRD3; NTSR1; GPR14; PGR8; DRD4; NTSR2; AVPR1A; PGR10; FZD1; DRD5; OPRD1; AVPR1B; PGR11; FZD2; FY; OPRK1; AVPR2; PGR12; FZD3; TG1019; OPRM1; OXTR; PGR13; FZD4; HM74; OPRL1; GPR48; PGR14; FZD5; GPR81; OPN1LW; GPR49; RDC1; FZD6; EDNRA; OPN1MW; LGR6; RE2; FZD7; EDNRB; OPN1SW; GPR27; RRH; FZD8; FPR1; RHO; GPR85; FZD9; FPRL1; HCRTR1; SREB3; FZD10; FPRL2; HCRTR2; GPR3; SMOH; FPR-RS1; PTAFR; GPR6; CALCR; FPR-RS2; PTGDR; GPR12; CALCRL; FPR-RS3; PTGER1; GPR21; CRHR1; FPR-RS4; PTGER2; GPR52; CRHR2; GALR1; PTGER3; GPR26; GIPR; TM7SF1; GALR2; PTGER4; GPR78; GCGR; TM7SF1L1; GALR3; PTGFR; GPR37; GLP1R; TM7SF1L2; GHSR; PTGIR; GPR37L1; GLP2R; TM7SF3; GPR38; TBXA2R; GPR41; GHRHR; TPRA40; and GPR43.

In some cases, a switch receptor is a receptor activated solely by a synthetic ligand (RASSL). A RASSL may be a GPCR designed to respond exclusively to a synthetic small molecule ligand. A RASSL may be comprised of any GPCR backbone, examples of which have been provided. In some cases, the RASSL is designed to be activated by a synthetic ligand. In this case, the RASSL may be not responsive or substantially less responsive to an endogenous ligand. Without wishing to be bound by theory, this method may provide temporal control of the RASSL such that the RASSL is only activated in the presence of the synthetic ligand. A RASSL may be less than 5%, less than 10%, less than 15%, less than 20%, less than 25%, less than 30%, less than 35%, less than 40%, less than 45%, less than 50%, less than 55%, less than 60%, less than 65%, less than 70%, less than 75%, less than 80%, less than 85%, less than 86%, less than 87%, less than 88%, less than 89%, less than 90%, less than 91%, less than 92%, less than 93%, less than 94%, less than 95%, less than 96%, less than 97%, less than 98%, less than 99% or less than 100% responsive to an endogenous ligand. In some cases, a RASSL is a fusion protein created from the joining of two or more genes (or portions of genes) that originally coded for separate proteins. In some cases, a RASSL is at least partially homologous to a wild-type GPCR. In some cases, a RASSL shares at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or at least 99.9% amino acid homology to a wild-type GPCR.

In some aspects, a switch receptor of the disclosure is a Designer Receptor Exclusively Activated by a Designer Drug (DREADD). In some cases, a DREADD is a RASSL. A DREADD may be any GPCR that is activated by a biologically inert ligand. The term “biologically inert” as used herein refers to any ligand (e.g., protein, small molecule, lipid, etc.) that has a low affinity for a wild-type receptor, yielding a ligand-receptor interaction with low responsiveness, but can have a high affinity for a switch receptor (e.g., a DREADD). Any ligand that is biologically inert will generally, at which a dose is typically delivered, have little to no physiological effect on an organism in the absence of the switch receptor. However, in the presence of the switch receptor, the biologically inert ligand may have a significant physiological effect on the organism (e.g., pain relief). The use of a biologically inert small molecule may be suitable to treat neurological disease by the methods disclosed herein due to e.g., a low risk of side effects and off-target effects. A DREADD may find particular utility to perform the methods described herein as the DREADD can be temporally controlled by a biologically inert molecule that otherwise has no effect on a subject. DREADDs may be designed using any GPCR backbone described above (e.g., via directed evolution or rational design). In some cases, the DREADD is not responsive or substantially less responsive to an endogenous ligand, such that it is predominately activated by a synthetic ligand. Examples of DREADDs include, without limitation, those designed on the muscarinic acetylcholine receptors (e.g., hM1D_{q}, hM2Dᵢ, hM3D_{q}, hM4Dᵢ and hM5D_{q}) as described by Armbruster et al., PNAS, 2007, and those designed on the kappa opioid receptor as described by Vardy et al., Neuron, 2015, the references of which are herein incorporated by reference. In some cases, the DREADD is activated by clozapine-N-oxide (e.g., hM1D_{q}, hM2Dᵢ, hM3D_{q}, hM4Dᵢ and hM5D_{q} receptors). In some cases, the biologically inert compound is a N4′-alkyl substituted CNO analog, such as any of the compounds disclosed in Chen et al., ACS Chem. Neurosci., 2015, including compound 4b (3-chloro-6-(4-ethylpiperazin-1-yl)-5H-benzo[b][1,4]benzodiazepine); compound 6 (4-(8-Chloro-5H-dibenzo[b,e][1,4]diazepin-11-yl)-1,1-dimethylpiperazin-1-ium iodide); compound 11 (3-chloro-6-(piperazin-1-yl)-5H-benzo[b][1,4]benzodiazepine); compound 13 (8-Chloro-11-[4-(1,1-dideutrioethyl)piperazin-1-yl]-5H-dibenzo[b,e][1,4]diazepine); and compound 21 (11-(Piperazin-1-yl)-5H-dibenzo[b,e][1,4]diazepine; 11-(4-Ethylpiperazin-1-yl)-5H-dibenzo[b,e][1,4]diazepine). In other cases, the DREADD is activated by salvinorin B (e.g., KOR-DREADD). It should be understood that any GPCR may be designed as a DREADD and the invention is not limited to the disclosed DREADDs. The synthetic ligand can be essentially any molecule that is biologically inert.

A DREADD may be selected as a switch receptor to perform the methods described herein based on the ability of the DREADD to activate a specific G protein and a specific signaling pathway. The signaling pathway may be chosen to specifically treat the neurological disease of interest. For example, the use of a DREADD that couples to the inhibitory G protein, Gᵢ, may be suitable to treat e.g., pain. One non-limiting example of a DREADD that couples to Gᵢ is hM4Dᵢ. In some cases, hM4Dᵢ is activated by clozapine-N-oxide. Other non-limiting examples of ligands that may activate hM4Dᵢ include clozapine, perlapine and olanzapine. In another example, a DREADD that couples to the excitatory G protein, G_{q}, may be suitable to treat e.g., pain. One non-limiting example of a DREADD that couples to G_{q} is hM3D_{q}. In some cases, hM3D_{q} is activated by clozapine-N-oxide, clozapine, perlapine or olanzapine.

Illustrative examples of GPCRs that specifically bind a ligand, RASSLs, and DREADDs may be derived from any GPCR including but not limited to: 5-Hydroxytryptamine receptors, Acetylcholine receptors (muscarinic), Adenosine receptors, Adhesion Class GPCRs, Adrenoceptors, Angiotensin receptors, Apelin receptor, Bile acid receptor, Bombesin receptors, Bradykinin receptors, Calcitonin receptors, Calcium-sensing receptors, Cannabinoid receptors, Chemerin receptor, Chemokine receptors, Cholecystokinin receptors, Class Frizzled GPCRs, Complement peptide receptors, Corticotropin-releasing factor receptors, Dopamine receptors, Endothelin receptors, Estrogen (G protein-coupled) receptor, Formylpeptide receptors, Free fatty acid receptors, GABAB receptors, Galanin receptors, Ghrelin receptor, Glucagon receptor family, Glycoprotein hormone receptors, Gonadotrophin-releasing hormone receptors, GPR18, GPR55 and GPR119, Histamine receptors, Hydroxycarboxylic acid receptors, Kisspeptin receptor, Leukotriene receptors, Lysophospholipid (LPA) receptors, Lysophospholipid (S1P) receptors, Melanin-concentrating hormone receptors, Melanocortin receptors, Melatonin receptors, Metabotropic glutamate receptors, Motilin receptor, Neuromedin U receptors, Neuropeptide FF/neuropeptide AF receptors, Neuropeptide S receptor, Neuropeptide W/neuropeptide B receptors, Neuropeptide Y receptors, Neurotensin receptors, Opioid receptors, Orexin receptors, Oxoglutarate receptor, P2Y receptors, Parathyroid hormone receptors, Peptide P518 receptor, Platelet-activating factor receptor, Prokineticin receptors, Prolactin-releasing peptide receptor, Prostanoid receptors, Proteinase-activated receptors, Relaxin family peptide receptors, Somatostatin receptors, Succinate receptor, Tachykinin receptors, Thyrotropin-releasing hormone receptors, Trace amine receptor, Urotensin receptor, Vasopressin and oxytocin receptors, and VIP and PACAP receptors.

Illustrative examples of GPCR-ligand pairs that are suitable for use in particular embodiments of treating neurological diseases contemplated herein are set forth in Table 1.

TABLE 1
Non-Limiting Examples of GPCR-Ligand Combinations
for the Treatment of Neurological Diseases

| Class | Receptor | Ligand | Effect | Mechanism |
|---|---|---|---|---|
| GPCR | AlstR | Allatostatin | Inhibition | Gi-coupled; |
| | (Drosophila) | | | activates GIRK |
| | | | | K+ channel |
| GPCR | DREADD | CNO, clozapine, | Inhibition | Gi-coupled; |
| | hM4Di (human) | perlapine, | | activates GIRK |
| | | olanzapine, | | K+ channel; |
| | | alosetron, | | inhibits CA++ |
| | | fluperlapine, | | channel; inhibits |
| | | N4′-alkyl | | cAMP |
| | | substituted CNO | | |
| | | analogs* | | |
| GPCR | DREADD | Salvinorin B | Inhibition | Gi-coupled; |
| | KORD (human) | | | activates GIRK |
| | | | | K+ channel; |
| | | | | inhibits Ca++ |
| | | | | channel; inhibits |
| | | | | cAMP |
| GPCR | DREADD | CNO, clozapine, | Excitation | Gq-coupled; |
| | hM3Dq (human) | perlapine, | | inhibits KCNQ |
| | | olanzapine, | | K+ channel |
| | | alosetron, | | |
| | | fluperlapine, | | |
| | | N4′-alkyl | | |
| | | substituted CNO | | |
| | | analogs* | | |
| GPCR | DREADD | CNO, clozapine, | Modulates | Gs-coupled, |
| | GsD | perlapine, | circadian clock, | cAMP |
| | | olanzapine, | regulates insulin | modulation |
| | | alosetron, | secretion; | |
| | | fluperlapine, | excitation | |
| | | N4′-alkyl | | |
| | | substituted CNO | | |
| | | analogs* | | |

*N4′-alkyl substituted clozapine-N-oxide (CNO) analogs described in Chen et al., ACS Chem. Neurosci. 2015, PMID 25587888 including: 3-chloro-6-(4-ethylpiperazin-1-yl)-5H-benzo[b][ 1,4]benzodiazepine, 4-(8-Chloro-5H-dibenzo[b,e][1,4]diazepin-11-yl)-1,1-dimethylpiperazin-1-ium iodide, 3-chloro-6-(piperazin-1-yl)-5H-benzo[b][1,4]benzodiazepine, 8-Chloro-11-[4-(1,1-dideutrioethyl)piperazin-1-yl]-5H-dibenzo[b,e][1,4]diazepine, 11-(Piperazin-1-yl)-5H-dibenzo[b,e][1,4]diazepine, or 11-(4-Ethylpiperazin-1-yl)-5H- dibenzo[b,e][1,4]diazepine.

2. Ligand-Gated Ion Channels

In particular embodiments, the switch receptor is a ligand-gated ion channel (LGIC) or mutein thereof. An LGIC can be any transmembrane protein that controls the flux of ions (e.g., Na⁺, K⁺, Ca⁺⁺, Cl⁻) across a cell membrane in response to the binding of a ligand. Without wishing to be bound by theory, activation of an LGIC may alter the electrophysiological activity of an excitable cell (i.e., depolarize, hyperpolarize). LGICs may control the flux of cations, anions or a combination of both across membranes. The LGIC may be selected based on the ion-selectivity of the channel. In some cases, the LGIC controls the flux of chloride ions (Cl⁻) across a membrane and may be suitable to treat e.g., pain. In some aspects, one or more of the subunits of an LGIC or mutein thereof have been engineered to specifically bind to a heterologous ligand, an exogenous ligand and/or a synthetic ligand. Illustrative examples of LGICs suitable for use in particular embodiments include, but are not limited to 5-HT3 receptors, Acid-sensing (proton-gated) ion channels (ASICs), Epithelial sodium channels (ENaC), GABAA receptors, Glycine receptors, Ionotropic glutamate receptors, IP3 receptor, Nicotinic acetylcholine receptors, P2X receptors, Ryanodine receptor, and Zinc activated channels (ZAC).

In some aspects, a ligand-gated ion channel comprises an ion conduction pore domain and ligand binding domain created by the fusion of two or more polynucleotide sequences that originally coded for separate polypeptides. In some embodiments, the polynucleotide sequences comprise two or more members of the cys loop receptor gene family. In one embodiment, the ion conduction pore domain conducts anions. In another embodiment, the ion conduction pore domain conducts cations.

In some cases, the LGIC is engineered or modified to be activatable by a synthetic ligand. Non-limiting examples of LGICs suitable to perform the methods as described herein include: a Glutamate-gated chloride channel engineered to respond to the synthetic ligand, ivermectin (Frazier et al., Journal of Biological Chemistry, 2012); a Pharmacologically-Selective Actuator Module (PSAM) activated by a Pharmacologically-Selective Effector Molecule (PSEM), including: PSAM-5HT3HC activated by the synthetic ligand PSEM22S, PSAM-GlyR activated by the synthetic ligand PSEM89S, and PSAM-nAChR activated by the synthetic ligand PSEM9S (Magnus et al., Science, 2011); TRPV1 activated by capsaicin; GlyR-M activated by the synthetic ligand ivermectin (Lynagh and Lynch, Journal of Biological Chemistry, 2010); and GABA-A activated by the synthetic ligand Zolpidem.

Other non-limiting examples of LGICs that are suitable for use with the methods described herein include: HTR3A; HTR3B; HTR3C; HTR3D; HTR3E; ASIC1; ASIC2; ASIC3; SCNN1A; SCNN1B; SCNN1D; SCNN1G; GABRA1; GABRA2; GABRA3; GABRA4; GABRA5; GABRA6; GABRB1; GABRB2; GABRB3; GABRG1; GABRG2; GABRG3; GABRD; GABRE; GABRQ; GABRP; GABRR1; GABRR2; GABRR3; GLRA1; GLRA2; GLRA3; GLRA4; GLRB; GRIA1; GRIA2; GRIA3; GRIA4; GRID1; GRID2; GRIK1; GRIK2; GRIK3; GRIK4; GRIK5; GRIN1; GRIN2A; GRIN2B; GRIN2C; GRIN2D; GRIN3A; GRIN3B; ITPR1; ITPR2; ITPR3; CHRNA1; CHRNA2; CHRNA3; CHRNA4; CHRNA5; CHRNA6; CHRNA7; CHRNA9; CHRNA10; CHRNB1; CHRNB2; CHRNB3; CHRNB4; CHRNG; CHRND; CHRNE; P2RX1; P2RX2; P2RX3; P2RX4; P2RX5; P2RX6; P2RX7; RYR1; RYR2; RYR3; and ZACN.

TRPV1, TRPM8 and P2X₂ are members of large LGIC families that share structural features as well as gating principles. For example TRPV4, similar to TRPV1, is also triggered by heat, but not by capsaicin; and P2X₃, is triggered by ATP, but desensitizes more rapidly than P2X₂. TRPV1, TRPM8 and P2X₂ are, therefore, non-limiting examples of LGIC suitable for use in particular embodiments.

In one embodiment, the switch receptor is a TRPV1 or TRPM8 receptor or a mutein thereof. TRPV1 and TRPM8, are vanilloid and menthol receptors expressed by nociceptive neurons of the peripheral nervous system. Both channels are thought to function as non-selective, sodium- and calcium-permeable homotetramers. In addition, both channels and their principal agonists—capsaicin and cooling compounds, such as menthol, respectively—are virtually absent from the central nervous system. Capsaicin and some cooling compounds, including menthol and icilin, contain potential acceptor sites for photolabile blocking groups. Association of a photolabile blocking group with such an acceptor would result in a ligand-gated ion channel in which light acts as an indirect trigger by releasing the active ligand.

In one embodiment, the switch receptor is a P2X₂ receptor or a mutein thereof. P2X₂ is an ATP-gated non-selective cation channel distinguished by its slow rate of desensitization. P2X₂ may be used as a selectively addressable source of depolarizing current and present a platform for the generation of engineered channel-ligand combinations that lack natural agonists altogether.

Illustrative examples of LGIC-ligand pairs that are suitable for use in particular embodiments of treating neurological diseases contemplated herein are set forth in Table 2.

TABLE 2
Non-Limiting Examples of LGIC-Ligand Combinations
for the Treatment of Neurological Diseases

| Class | Receptor | Ligand | Effect | Mechanism |
|---|---|---|---|---|
| LGIC | GluCl α and β | Ivermectin, | Inhibition | Cl− channel |
| | (C. elegans) | selamectin, | | |
| | | doramectin, | | |
| | | emamectin, | | |
| | | eprinomectin, | | |
| | | abamectin, | | |
| | | moxidectin | | |
| LGIC | PSAM- | PSEM^{22S} | Excitation | Cation channel |
| | 5HT3HC | | | |
| | (human-mouse) | | | |
| LGIC | PSAM-GlyR | PSEM^{89S} | Inhibition | Cl− channel |
| LGIC | PSAM-nAChR | PSEM^{9S} | Not shown | Ca++ channel |
| LGIC | TRPV1 (rat) | Capsaicin | Excitation | Cation channel |
| LGIC | GlyR-M | Ivermectin, | Inhibition | Cl− channel |
| | (human) | selamectin, | | |
| | | doramectin, | | |
| | | emamectin, | | |
| | | eprinomectin, | | |
| | | abamectin, | | |
| | | moxidectin | | |
| LGIC | GABAA | Zolpidem | Inhibition | Cl− channel |
| | (mouse) | | | |

3. Glycine Receptors

In particular embodiments, the switch receptor is a Glycine receptor (GlyR) or mutein thereof. In some aspects, one or more of the subunits of a GlyR or mutein thereof have been engineered to specifically bind to a heterologous ligand, an exogenous ligand and/or a synthetic ligand. The GlyR is a member of the nicotinicoid superfamily of ligand-gated ionotropic receptors that mediate fast neurotransmission in the central nervous system (CNS). Heterologous expression of just the human α1 subunit, however, is sufficient to reconstitute an active glycine-gated channel with pharmacological properties essentially identical to those of native channels. Accordingly, in various illustrative embodiments, the switch receptor comprises a subunit of GlyR (e.g., alpha1, alpha2, alpha3, alpha4, or beta), and preferably comprises a subunit of mammalian origin or a mutein of such subunit. Illustrative examples of GlyR suitable for use in particular embodiments, are described in U.S. Pat. No. 8,957,036, which is incorporated by reference herein in its entirety.

Mutant forms of GlyR subunits with altered activity (muteins) also are known, and can be used in particular embodiments. For example, certain muteins of GlyR proteins result in altered ion-channel properties, such as resulting in a cationic ion channel (e.g., Δ250A251E; Keramidas et al., J. Gen. Physiol., 119, 393 (2002)). Other GlyR muteins lack sites for zinc potentiation or zinc inhibition (Hirzel et al., Neuron, 52, 679-90 (2006)), affinity for allosteric modulators (e.g., anesthetic potentiation (Hemmings et al., Trends Pharmacol. Sci., 26, 503-10 (2005)), or affinity for ligands (Rajendra et al., Neuron, 14, 169-175 (1995); Schrnieden et al., Science, 262, 256-258 (1993)). Mutation of GlyR subunits also can selectively alter ion permeation (e.g., anionic- or cationic-selective channels), and redesign a receptor subunit's ligand binding pockets to recognize heterologous or synthetic ligands. For example, to alter the sensitivity and selectivity of a GlyR protein for a particular ligand, point mutations can be made in the GlyRα1 subunit that are expected to shift the dose response curve to the left or right (i.e., less or more specific to glycine). Other mutations can alter the sensitivity of a GlyR protein to certain anesthetics (e.g., ethanol). For example, a mutation in the mouse glycineal receptor subunit in which a methionine (M) at position 287 is changed to leucine (L) (M297L) results in greatly enhanced sensitivity to the volatile anesthetic enflurane. Such GlyR muteins can be employed as the GlyR protein in particular embodiments.

In particular preferred embodiments, the switch receptor comprises a GlyRα1 subunit comprising one or more amino acid deletions, insertions, or substitutions that abolish GlyR binding to its natural ligand and confer specific binding of the GlyR mutein to a heterologous or synthetic ligand. In one preferred embodiment, the switch receptor comprises a GlyRα1 subunit comprising amino acid insertions, deletions, or substitutions in F207 and/or A228. In one preferred embodiment, the switch receptor comprises a GlyRα1 subunit comprising amino acid substitutions F207A and/or A228G. In yet another preferred embodiment, the switch receptor comprises a GlyRα1 subunit comprising amino acid substitutions F207A and A228G and specifically binds the ligand ivermectin.

In yet another preferred embodiment, the switch receptor comprises a GlyRα1 subunit comprising amino acid substitutions F207A and A228G and specifically binds avermectins (as a broad class) including the ivermectin analogs selamectin, doramectin, emamectin, eprinomectin, and abamectin in addition to moxidectin (a milbemycin) and analogs thereof.

In some embodiments, a switch receptor comprises a GlyRα1 subunit comprising one or more of the following amino acid substitutions: A-1′E, P-2′Δ, T13′V, R19′E, F207A, and A228G (see, Islam et al., ACS Chem. Neurosci., DOI: 10.1021/acschemneuro.6b00168 (2016)). In one embodiment, a switch receptor comprises a GlyRα1 subunit comprising amino acid substitutions A-FE, F207A, and A228G and specifically binds the ligand ivermectin. In another embodiment, a switch receptor comprises a GlyRα1 subunit comprising amino acid substitutions A-1′E, P-2′Δ, T13′V, F207A, and A228G and specifically binds the ligand ivermectin.

Illustrative examples of avermectin analogs suitable for use in particular embodiments contemplated herein include, but are not limited to existing analogs in the PubChem Compound Database, which can be found at the National Center for Biotechnology Information web site.

### E. Ligands

Ligands suitable to treat neurological diseases (e.g., pain) can include any molecule that can activate a switch receptor as described herein. A ligand can be a nucleic acid, a small molecule compound, a protein or peptide, a lipid, a photon and the like. Non-limiting examples of ligands suitable for activating a GPCR-derived switch receptor of the disclosure include: allatostatin; nalfurafine (C₂₈H₃₂N₂O₅), clozapine-N-oxide (CNO); clozapine; olanzapine; perlapine; salvinorin B; alosetron; fluperlapine; and N4′-alkyl substituted CNO analogs as disclosed in Chen et al., ACS Chem. Neurosci., 2015, including compound 4b (3-chloro-6-(4-ethylpiperazin-1-yl)-5H-benzo[b][1,4]benzodiazepine); compound 6 (4-(8-Chloro-5H-dibenzo[b,e][1,4]diazepin-11-yl)-1,1-dimethylpiperazin-1-ium iodide); compound 11 (3-chloro-6-(piperazin-1-yl)-5H-benzo[b][1,4]benzodiazepine); compound 13 (8-Chloro-11-[4-(1,1-dideutrioethyl)piperazin-1-yl]-5H-dibenzo[b,e][1,4]diazepine); and compound 21 (11-(Piperazin-1-yl)-5H-dibenzo[b,e][1,4]diazepine; 11-(4-Ethylpiperazin-1-yl)-5H-dibenzo[b,e][1,4]diazepine). Non-limiting examples of ligands suitable for activating an LGIC-derived switch receptor of the disclosure include: members of the avermectin family including: ivermectin, selamectin, doramectin, emamectin, eprinomectin, and abamectin; members of the milbemycin family including: milbemectin, moxidectin and nemadectin; imidazopyridines including: zolpidem, alpidem, saripidem, necopidem, fasiplon and DS-1; capsaicinoids including: capsaicin, dihydrocapsaicin, nordihydrocapsaicin, homodihydrocapsaicin, homocapsaicin and nonivamide; Pharmacologically-Selective Effector Molecules (PSEM) including: PSEM22S, PSEM89S, and PSEM9S; and ATP. In some cases, a ligand binding domain is activated by the binding of clozapine-N-oxide, clozapine, perlapine, olanzapine, alosetron, fluperlapine, nalfurafine (C₂₈H₃₂N₂O₅), or N4′-alkyl substituted CNO analogs. In certain aspects, a ligand binding domain is activated by the binding of nicotine, varenicline, or galantamine. In some cases, the ligand is not glycine, beta-alanine or taurine.

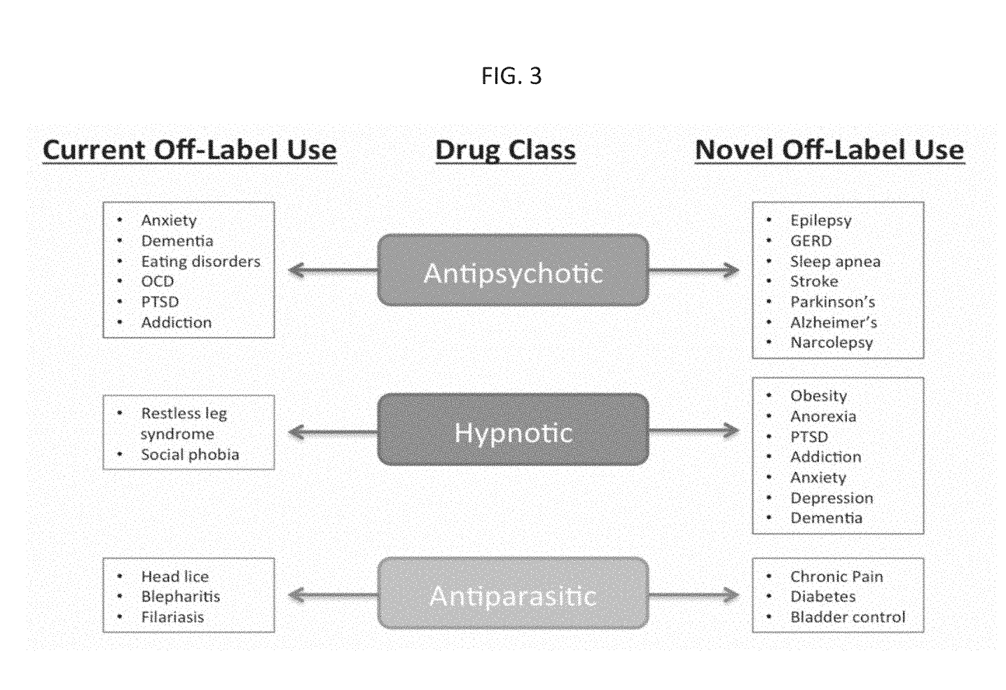

In some cases, a ligand is a drug that is FDA-approved for one or more indications, not including the neurological disease or disorder envisioned herein. Put another way, a subject suffering from a neurological disorder may be treated with an FDA-approved drug that is not FDA-approved to treat the neurological disorder (i.e., “off-label” indication). FIG. 3 provides non-limiting examples of FDA-approved drugs that may be repurposed to treat a neurological disorder for which that drug is not FDA-approved. For example, clozapine is, at the time of this filing, FDA-approved for the treatment of schizophrenia and is also used “off-label” for the treatment of anxiety disorders. It is envisioned that clozapine can be repurposed to treat, for example, gastroesophageal reflux disorder (GERD), using the compositions and methods described herein. In another non-limiting example, perlapine is a hypnotic that could be repurposed for the treatment of obesity. In yet another non-limiting example, ivermectin is an anti-parasitic that could be repurposed for the treatment of chronic pain.

### F. Polynucleotides

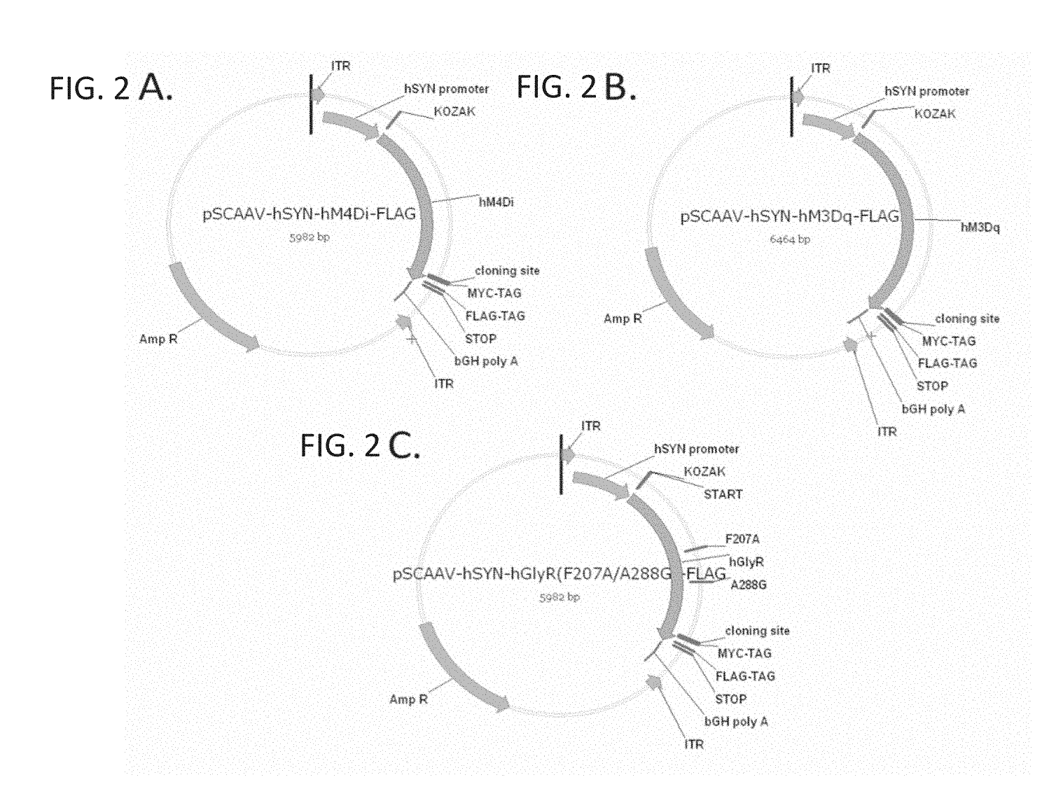

In various illustrative embodiments, the present invention contemplates, in part, polynucleotides, polynucleotides encoding switch receptor polypeptides including, but not limited to GPCRs, RASSLs, DREADDs, LGICs, and subunits and muteins thereof, and fusion polypeptides, viral vector polynucleotides, and compositions comprising the same. See, e.g., SEQ ID NO: 1 (Table 4) and FIGS. 2A-2C.

As used herein, the terms “polynucleotide,” “nucleotide,” “nucleotide sequence” or “nucleic acid” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. Polynucleotides may be deoxyribonucleic acid (DNA), ribonucleic acid (RNA) or DNA/RNA hybrids. Polynucleotides may be single-stranded or double-stranded. Polynucleotides include, but are not limited to: pre-messenger RNA (pre-mRNA), messenger RNA (mRNA), RNA, short interfering RNA (siRNA), short hairpin RNA (shRNA), microRNA (miRNA), ribozymes, synthetic RNA, genomic RNA (gRNA), plus strand RNA (RNA(+)), minus strand RNA (RNA(−)), synthetic RNA, genomic DNA (gDNA), PCR amplified DNA, complementary DNA (cDNA), synthetic DNA, or recombinant DNA. Polynucleotides refer to a polymeric form of nucleotides of at least 5, at least 10, at least 15, at least 20, at least 25, at least 30, at least 40, at least 50, at least 100, at least 200, at least 300, at least 400, at least 500, at least 1000, at least 5000, at least 10000, or at least 15000 or more nucleotides in length, either ribonucleotides or deoxynucleotides or a modified form of either type of nucleotide, as well as all intermediate lengths. It will be readily understood that “intermediate lengths,” in this context, means any length between the quoted values, such as 6, 7, 8, 9, etc., 101, 102, 103, etc.; 151, 152, 153, etc.; 201, 202, 203, etc. In particular embodiments, polynucleotides or variants have at least or about 50%, 55%, 60%, 65%, 70%, 71%, 72%, 73%, 74%, 75%,76%, 77%, 78%, 79%, 80%, 81%, 82%, 83%, 84%,85%, 86%, 87%, 88%, 89%, 90%, 91%, 92%, 93%, 94%, 95%, 96%, 97%, 98%, 99% or 100% sequence identity to a reference sequence described herein or known in the art, typically where the variant maintains at least one biological activity of the reference sequence.

As used herein, the term “gene” may refer to a polynucleotide sequence comprising enhancers, promoters, introns, exons, and the like. In particular embodiments, the term “gene” refers to a polynucleotide sequence encoding a polypeptide, regardless of whether the polynucleotide sequence is identical to the genomic sequence encoding the polypeptide.

A “genomic sequence regulating transcription of” or a “genomic sequence that regulates transcription or” refers to a polynucleotide sequence that is associated with the transcription of a gene. In one embodiment, the genomic sequence regulates transcription because it is a binding site for a polypeptide that represses or decreases transcription or a polynucleotide sequence associated with a transcription factor binding site that contributes to transcriptional repression.

A “cis-acting sequence regulating transcription of” or a “cis-acting nucleotide sequence that regulates transcription or” or equivalents refers to a polynucleotide sequence that is associated with the transcription of a gene. In one embodiment, the cis-acting sequence regulates transcription because it is a binding site for a polypeptide that represses or decreases transcription or a polynucleotide sequence associated with a transcription factor binding site that contributes to transcriptional repression.

A “regulatory element” or “cis-acting sequence” or equivalents thereof refer to an expression control sequence that comprises a polynucleotide sequence that is associated with the transcription or expression of a polynucleotide sequence encoding a polypeptide.

A “regulatory element for inducible expression” refers to a polynucleotide sequence that is a promoter, enhancer, or functional fragment thereof that is operably linked to a polynucleotide to be expressed. The regulatory element for inducible expression responds to the presence or absence of a molecule that binds the element to increase (turn-on) or decrease (turn-off) the expression of the polynucleotide operably linked thereto. Illustrative regulatory elements for inducible expression include, but are not limited to, a tetracycline responsive promoter, an ecdysone responsive promoter, a cumate responsive promoter, a glucocorticoid responsive promoter, an estrogen responsive promoter, an RU-486 responsive promoter, a PPAR-γ promoter, and a peroxide inducible promoter.

A “regulatory element for transient expression” refers to a polynucleotide sequence that can be used to briefly or temporarily express a polynucleotide nucleotide sequence. In particular embodiments, one or more regulatory elements for transient expression can be used to limit the duration of a polynucleotide. In certain embodiments, the preferred duration of polynucleotide expression is on the order of minutes, hours, or days. Illustrative regulatory elements for transient expression include, but are not limited to, nuclease target sites, recombinase recognition sites, and inhibitory RNA target sites. In addition, to some extent, in particular embodiments, a regulatory element for inducible expression may also contribute to controlling the duration of polynucleotide expression.

As used herein, the terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms also encompass polynucleotides that are distinguished from a reference polynucleotide by the addition, deletion, substitution, or modification of at least one nucleotide. Accordingly, the terms “polynucleotide variant” and “variant” include polynucleotides in which one or more nucleotides have been added or deleted, or modified, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide.

In one embodiment, a polynucleotide comprises a nucleotide sequence that hybridizes to a target nucleic acid sequence under stringent conditions. To hybridize under “stringent conditions” describes hybridization protocols in which nucleotide sequences at least 60% identical to each other remain hybridized. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium.

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Terms used to describe sequence relationships between two or more polynucleotides or polypeptides include “reference sequence,” “comparison window,” “sequence identity,” “percentage of sequence identity,” and “substantial identity.” A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., Current Protocols in Molecular Biology, John Wiley & Sons Inc, 1994-1998, Chapter 15.

An “isolated polynucleotide,” as used herein, refers to a polynucleotide that has been purified from the sequences which flank it in a naturally-occurring state, e.g., a DNA fragment that has been removed from the sequences that are normally adjacent to the fragment. In particular embodiments, an “isolated polynucleotide” refers to a complementary DNA (cDNA), a recombinant DNA, or other polynucleotide that does not exist in nature and that has been made by the hand of man.

Terms that describe the orientation of polynucleotides include: 5′ (normally the end of the polynucleotide having a free phosphate group) and 3′ (normally the end of the polynucleotide having a free hydroxyl (OH) group). Polynucleotide sequences can be annotated in the 5′ to 3′ orientation or the 3′ to 5′ orientation. For DNA and mRNA, the 5′ to 3′ strand is designated the “sense,” “plus,” or “coding” strand because its sequence is identical to the sequence of the pre-messenger (premRNA) [except for uracil (U) in RNA, instead of thymine (T) in DNA]. For DNA and mRNA, the complementary 3′ to 5′ strand which is the strand transcribed by the RNA polymerase is designated as “template,” “antisense,” “minus,” or “non-coding” strand. As used herein, the term “reverse orientation” refers to a 5′ to 3′ sequence written in the 3′ to 5′ orientation or a 3′ to 5′ sequence written in the 5′ to 3′ orientation.

The term “flanked” refers to a polynucleotide sequence that is in between an upstream polynucleotide sequence and/or a downstream polynucleotide sequence, i.e., 5′ and/or 3′, relative to the sequence. For example, a sequence that is “flanked” by two other elements (e.g., ITRs), indicates that one element is located 5′ to the sequence and the other is located 3′ to the sequence; however, there may be intervening sequences therebetween.

The terms “complementary” and “complementarity” refer to polynucleotides (i.e., a sequence of nucleotides) related by the base-pairing rules. For example, the complementary strand of the DNA sequence 5′ A G T C A T G 3′ is 3′ T C A G T A C 5′. The latter sequence is often written as the reverse complement with the 5′ end on the left and the 3′ end on the right, 5′ C A T G A C T 3′. A sequence that is equal to its reverse complement is said to be a palindromic sequence. Complementarity can be “partial,” in which only some of the nucleic acids' bases are matched according to the base pairing rules. Or, there can be “complete” or “total” complementarity between the nucleic acids.

The terms “nucleic acid cassette” or “expression cassette” as used herein refers to polynucleotide sequences within a larger polynucleotide, such as a vector, which are sufficient to express one or more RNAs from a polynucleotide. The expressed RNAs may be translated into proteins, may function as guide RNAs or inhibitory RNAs to target other polynucleotide sequences for cleavage and/or degradation. In one embodiment, the nucleic acid cassette contains one or more polynucleotide(s)-of-interest. In another embodiment, the nucleic acid cassette contains one or more expression control sequences operably linked to one or more polynucleotide(s)-of-interest. Polynucleotides include polynucleotide(s)-of-interest. As used herein, the term “polynucleotide-of-interest” refers to a polynucleotide encoding a polypeptide or fusion polypeptide or a polynucleotide that serves as a template for the transcription of an inhibitory polynucleotide, e.g., GPCRs, RASSLs, DREADDs, LGICs, and subunits and muteins thereof, as contemplated herein. In a particular embodiment, a polynucleotide-of-interest encodes a polypeptide or fusion polypeptide having one or more enzymatic activities, such as a nuclease activity and/or chromatin remodeling or epigenetic modification activities.

Vectors may comprise 1, 2, 3, 4, 5, 6, 7, 8, 9 or 10 or more nucleic acid cassettes. In a preferred embodiment of the invention, a nucleic acid cassette comprises one or more expression control sequences (e.g., a promoter or enhancer operable in a neuronal cell) operably linked to a polynucleotide encoding a switch receptor, e.g., a GPCR, RASSL, DREADD, LGIC, or subunit or muteins thereof. The cassette can be removed from or inserted into other polynucleotide sequences, e.g., a plasmid or viral vector, as a single unit.

In one embodiment, a polynucleotide contemplated herein comprises 1, 2, 3, 4, 5, 6, 7, 8, 9, or more nucleic acid cassettes any number or combination of which may be in the same or opposite orientations.

Moreover, it will be appreciated by those of ordinary skill in the art that, as a result of the degeneracy of the genetic code, there are many nucleotide sequences that may encode a polypeptide, or fragment of variant thereof, as contemplated herein. Some of these polynucleotides bear minimal homology to the nucleotide sequence of any native gene. Nonetheless, polynucleotides that vary due to differences in codon usage are specifically contemplated by the present invention, for example polynucleotides that are optimized for human and/or primate codon selection. In one embodiment, polynucleotides comprising particular allelic sequences are provided. Alleles are endogenous polynucleotide sequences that are altered as a result of one or more mutations, such as deletions, additions and/or substitutions of nucleotides.

In a certain embodiment, a polynucleotide-of-interest encodes an inhibitory polynucleotide including, but not limited to an siRNA, an miRNA, an shRNA, a ribozyme or another inhibitory RNA, or system for inhibiting RNA, e.g., a CRISPR/CAS9 system. In particular embodiments, the polynucleotide-of-interest is an inhibitory RNA that targets a molecule that is associated with an increased sensitivity to pain, e.g., TNFα, Nav1.1, Nav1.3, Nav1.6, Nav1.7, Nav1.8, Nav1.9, TRPV1, TRPV2, TRPV3, TRPV4, TRPC, TRPP, ACCN1, ACCN2, TRPM8, TRPA1, P2XR3, P2RY, BDKRB1, BDKRB2, Htr3A, ACCNs, KCNQ, HCN2, HCN4, CSF-1, CACNA1A-S, CACNA2D1, IL1, IL6, IL12, IL18, COX-2, NTRK1, NGF, GDNF, LIF, CCL2, CNR2, TLR2, TLR4, P2RX4, P2RX7, CCL2, CX3CR1, and BDNF.

As used herein, the terms “siRNA” or “short interfering RNA” refer to a short polynucleotide sequence that mediates a process of sequence-specific post-transcriptional gene silencing, translational inhibition, transcriptional inhibition, or epigenetic RNAi in animals (Zamore et al., 2000, Cell, 101, 25-33; Fire et al., 1998, Nature, 391, 806; Hamilton et al., 1999, Science, 286, 950-951; Lin et al., 1999, Nature, 402, 128-129; Sharp, 1999, Genes & Dev., 13, 139-141; and Strauss, 1999, Science, 286, 886). In certain embodiments, an siRNA comprises a first strand and a second strand that have the same number of nucleosides; however, the first and second strands are offset such that the two terminal nucleosides on the first and second strands are not paired with a residue on the complimentary strand. In certain instances, the two nucleosides that are not paired are thymidine resides. The siRNA should include a region of sufficient homology to the target gene, and be of sufficient length in terms of nucleotides, such that the siRNA, or a fragment thereof, can mediate down regulation of the target gene. Thus, an siRNA includes a region which is at least partially complementary to the target RNA. It is not necessary that there be perfect complementarity between the siRNA and the target, but the correspondence must be sufficient to enable the siRNA, or a cleavage product thereof, to direct sequence specific silencing, such as by RNAi cleavage of the target RNA. Complementarity, or degree of homology with the target strand, is most critical in the antisense strand. While perfect complementarity, particularly in the antisense strand, is often desired, some embodiments include one or more, but preferably 10, 8, 6, 5, 4, 3, 2, or fewer mismatches with respect to the target RNA. The mismatches are most tolerated in the terminal regions, and if present are preferably in a terminal region or regions, e.g., within 6, 5, 4, or 3 nucleotides of the 5′ and/or 3′ terminus. The sense strand need only be sufficiently complementary with the antisense strand to maintain the overall double-strand character of the molecule. Each strand of an siRNA can be equal to or less than 30, 25, 24, 23, 22, 21, or 20 nucleotides in length. The strand is preferably at least 19 nucleotides in length. For example, each strand can be between 21 and 25 nucleotides in length. Preferred siRNAs have a duplex region of 17, 18, 19, 29, 21, 22, 23, 24, or 25 nucleotide pairs, and one or more overhangs of 2-3 nucleotides, preferably one or two 3′ overhangs, of 2-3 nucleotides.

As used herein, the terms “miRNA” or “microRNA” s refer to small non-coding RNAs of 20-22 nucleotides, typically excised from ˜70 nucleotide foldback RNA precursor structures known as pre-miRNAs. miRNAs negatively regulate their targets in one of two ways depending on the degree of complementarity between the miRNA and the target. First, miRNAs that bind with perfect or nearly perfect complementarity to protein-coding mRNA sequences induce the RNA-mediated interference (RNAi) pathway. miRNAs that exert their regulatory effects by binding to imperfect complementary sites within the 3′ untranslated regions (UTRs) of their mRNA targets, repress target-gene expression post-transcriptionally, apparently at the level of translation, through a RISC complex that is similar to, or possibly identical with, the one that is used for the RNAi pathway. Consistent with translational control, miRNAs that use this mechanism reduce the protein levels of their target genes, but the mRNA levels of these genes are only minimally affected. miRNAs encompass both naturally occurring miRNAs as well as artificially designed miRNAs that can specifically target any mRNA sequence. For example, in one embodiment, the skilled artisan can design short hairpin RNA constructs expressed as human miRNA (e.g., miR-30 or miR-21) primary transcripts or “mishRNA.” This design adds a Drosha processing site to the hairpin construct and has been shown to greatly increase knockdown efficiency (Pusch et al., 2004). The hairpin stem consists of 22-nt of dsRNA (e.g., antisense has perfect complementarity to desired target) and a 15-19-nt loop from a human miR. Adding the miR loop and miR30 flanking sequences on either or both sides of the hairpin results in greater than 10-fold increase in Drosha and Dicer processing of the expressed hairpins when compared with conventional shRNA designs without microRNA. Increased Drosha and Dicer processing translates into greater siRNA/miRNA production and greater potency for expressed hairpins.

As used herein, the terms “shRNA” or “short hairpin RNA” refer to double-stranded structure that is formed by a single self-complementary RNA strand. shRNA constructs containing a nucleotide sequence identical to a portion, of either coding or non-coding sequence, of the target gene are preferred for inhibition. RNA sequences with insertions, deletions, and single point mutations relative to the target sequence have also been found to be effective for inhibition. Greater than 90% sequence identity, or even 100% sequence identity, between the inhibitory RNA and the portion of the target gene is preferred. In certain preferred embodiments, the length of the duplex-forming portion of an shRNA is at least 20, 21 or 22 nucleotides in length, e.g., corresponding in size to RNA products produced by Dicer-dependent cleavage. In certain embodiments, the shRNA construct is at least 25, 50, 100, 200, 300 or 400 bases in length. In certain embodiments, the shRNA construct is 400-800 bases in length. shRNA constructs are highly tolerant of variation in loop sequence and loop size.

As used herein, the term “ribozyme” refers to a catalytically active RNA molecule capable of site-specific cleavage of target mRNA. Several subtypes have been described, e.g., hammerhead and hairpin ribozymes. Ribozyme catalytic activity and stability can be improved by substituting deoxyribonucleotides for ribonucleotides at noncatalytic bases. While ribozymes that cleave mRNA at site-specific recognition sequences can be used to destroy particular mRNAs, the use of hammerhead ribozymes is preferred. Hammerhead ribozymes cleave mRNAs at locations dictated by flanking regions that form complementary base pairs with the target mRNA. The sole requirement is that the target mRNA has the following sequence of two bases: 5′-UG-3′. The construction and production of hammerhead ribozymes is well known in the art.

The polynucleotides contemplated herein, regardless of the length of the coding sequence itself, may be combined with other DNA sequences, such as expression control sequences, regulatory elements, promoters and/or enhancers, untranslated regions (UTRs), Kozak sequences, polyadenylation signals, additional restriction enzyme sites, multiple cloning sites, internal ribosomal entry sites (IRES), recombinase recognition sites (e.g., LoxP, FRT, and Att sites), guide RNA target sites, termination codons, transcriptional termination signals, and polynucleotides encoding self-cleaving polypeptides, epitope tags, as disclosed elsewhere herein or as known in the art, such that their overall length may vary considerably. It is therefore contemplated that a polynucleotide fragment of almost any length may be employed, with the total length preferably being limited by the ease of preparation and use in the intended recombinant DNA protocol.

Polynucleotides can be prepared, manipulated and/or expressed using any of a variety of well-established techniques known and available in the art. In order to express a desired polypeptide, a nucleotide sequence encoding the polypeptide, can be inserted into an appropriate vector, such as a viral vector. In preferred embodiments, the viral vector is an adeno-associated virus (AAV) vector.

“Expression control sequences,” “control elements,” or “regulatory sequences” present in an expression vector are those non-translated regions of the vector—origin of replication, selection cassettes, promoters, enhancers, translation initiation signals (Shine Dalgarno sequence or Kozak sequence) introns, a polyadenylation sequence, 5′ and 3′ untranslated regions—which interact with host cellular proteins to carry out transcription and translation. Such elements may vary in their strength and specificity. Depending on the vector system and host utilized, any number of suitable transcription and translation elements, including ubiquitous promoters and inducible promoters may be used.

In particular embodiments, a polynucleotide for use in practicing the invention is a vector, including but not limited to expression vectors and viral vectors, and includes exogenous, endogenous, or heterologous control sequences such as promoters and/or enhancers. An “endogenous” control sequence is one which is naturally linked with a given gene in the genome. An “exogenous” control sequence is one which is placed in juxtaposition to a gene by means of genetic manipulation (i.e., molecular biological techniques) such that transcription of that gene is directed by the linked enhancer/promoter. A “heterologous” control sequence is an exogenous sequence that is from a different species than the cell being genetically manipulated.

The term “promoter” as used herein refers to a recognition site of a polynucleotide (DNA or RNA) to which an RNA polymerase binds. An RNA polymerase initiates and transcribes polynucleotides operably linked to the promoter. In particular embodiments, promoters operative in mammalian cells comprise an AT-rich region located approximately 25 to 30 bases upstream from the site where transcription is initiated and/or another sequence found 70 to 80 bases upstream from the start of transcription, a CNCAAT region where N may be any nucleotide. In particular embodiments, the vector comprises one or more RNA pol II and/or RNA pol III promoters.

Illustrative examples of RNA pol II promoters suitable for use in particular embodiments include, but are not limited to a neuron specific promoter.

The term “enhancer” refers to a segment of DNA which contains sequences capable of providing enhanced transcription and in some instances can function independent of their orientation relative to another control sequence. An enhancer can function cooperatively or additively with promoters and/or other enhancer elements. The term “promoter/enhancer” refers to a segment of DNA which contains sequences capable of providing both promoter and enhancer functions.

The term “operably linked”, refers to a juxtaposition wherein the components described are in a relationship permitting them to function in their intended manner. In one embodiment, the term refers to a functional linkage between a nucleic acid expression control sequence (such as a promoter, and/or enhancer) or regulatory element and a second polynucleotide sequence, e.g., a polynucleotide-of-interest, wherein the expression control sequence or regulatory element directs transcription of the nucleic acid corresponding to the second sequence.

As used herein, the term “constitutive expression control sequence” refers to a promoter, enhancer, or promoter/enhancer that continually or continuously allows for transcription of an operably linked sequence. A constitutive expression control sequence may be a “ubiquitous” promoter, enhancer, or promoter/enhancer that allows expression in a wide variety of cell and tissue types or a “cell specific,” “cell type specific,” “cell lineage specific,” or “tissue specific” promoter, enhancer, or promoter/enhancer that allows expression in a restricted variety of cell and tissue types, respectively.

Illustrative ubiquitous expression control sequences suitable for use in particular embodiments of the invention include, but are not limited to, a cytomegalovirus (CMV) immediate early promoter, a viral simian virus 40 (SV40) (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus (Irions et al., Nature Biotechnology 25, 1477-1482 (2007)), a Ubiquitin C promoter (UBC), a phosphoglycerate kinase-1 (PGK) promoter, and a cytomegalovirus enhancer/chicken β-actin (CAG) promoter.

The compositions and methods described herein can be utilized for the selective expression of a switch receptor in a cell or tissue. The terms “selective expression” and “target-specific expression” may be used interchangeably herein and refer to the expression of a protein or nucleic acid in a specific cell or tissue type. Selective expression may involve the use of one or more promoters. The nucleic acid molecule encoding the switch receptor may include one or more promoters that direct expression of the switch receptor to a particular cell or tissue type.

In a particular embodiment, it may be desirable to use a tissue-specific promoter to achieve cell type specific, lineage specific, or tissue-specific expression of a desired polynucleotide sequence. According to certain embodiments, the cell type specific promoter is specific for cell types found in the brain (e.g., neurons, glial cells). Illustrative examples of tissue specific promoters include, but are not limited to: a glial fibrillary acidic protein (GFAP) promoter (astrocyte expression), a synapsin promoter (neuron expression), and calcium/calmodulin-dependent protein kinase II (neuron expression), tubulin alpha I (neuron expression), neuron-specific enolase (neuron expression), platelet-derived growth factor beta chain (neuron expression), a TRPV1 promoter (neuron expression), a Nav1.7 promoter (neuron expression), a Nav1.8 promoter (neuron expression), a Nav1.9 promoter (neuron expression), or an Advillin promoter (neuron expression).

In some cases, a switch receptor is selectively expressed in one or more neurons or group of neurons. Selective expression in one or more neurons may involve the use of one or more neuron-specific promoters. Non-limiting examples of neuron-specific promoters include: human synapsin-1 (SYN-1) promoter, calcium-calmodulin dependent protein kinase IIA (CaMKIIA) promoter, tubulin alpha 1 promoter, neuron-specific enolase (NSE) promoter, platelet-derived growth factor beta chain promoter (PDGFB), TRPV1 promoter, Nav1.7 promoter, Nav1.8 promoter, Nav1.9 promoter, Advillin promoter, the Drosophila single-minded homolog 1 (SIM1) promoter, oxytocin (OXT) promoter, Agouti-related peptide (AgRP) promoter, protein kinase C-delta (PKC-delta) promoter or ghrelin promoter. In some cases, the switch receptor is selectively expressed in a sensory neuron. In some cases, the switch receptor is selectively expressed in a dorsal root ganglion, a trigeminal ganglion, an A-beta fiber, an A-delta fiber, a C-fiber, a TRPV1+ neuron, a Nav1.7+ neuron, a Nav1.8+ neuron, or a Nav1.9+ neuron. Other exemplary examples include, without limitation, the vagus nerve, proopiomelanocortin (POMC) neurons, the paraventricular nucleus (PVH) of the hypothalamus, the arcuate nucleus of the hypothalamus, the lateral subdivision of the amygdala central nucleus, the C6 stellate ganglion, the lower esophageal sphincter vagus nerve, the myenteric plexus, the subthalamic nucleus (STN) and the like. The switch receptor can be expressed in one or more interneurons, excitatory neurons, or inhibitory neurons. In some examples, the switch receptor, and in particular LGIC-derived switch receptors, may be expressed in one or more excitable cells or group of excitable cells. An excitable cell is any cell that experiences fluctuations in the membrane potential as a result of ion flux across the cell membrane. Excitable cells can include neuronal cells, myocytes, and the like.

In some cases, a switch receptor is constitutively expressed (i.e., expressed continuously; non-specific expression). In these examples, expression of the switch receptor may be controlled by selective delivery or administration of a vector directly to a specific cell or tissue type. For example, a vector encoding a switch receptor under the control of a constitutive promoter may be delivered directly to a dorsal root ganglion or a trigeminal neuron. In some cases, widespread expression of a switch receptor may be achieved by e.g., systemic administration of a vector encoding a switch receptor under the control of a constitutive promoter. Non-limiting examples of suitable constitutive promoters include: cytomegalovirus (CMV) immediate early promoter, simian virus 40 (SV40) promoter, Moloney murine leukemia virus (MMLV) LTR promoter, Rous sarcoma virus (RSV) LTR, a herpes simplex virus (HSV) thymidine kinase promoter, H5 promoter from vaccinia virus, P7.5 promoter from vaccinia virus, P11 promoter from vaccinia virus, elongation factor 1-alpha (EF1a) promoter, early growth response 1 (EGR1) promoter, ferritin H (FerH) promoter, ferritin L (FerL) promoter, glyceraldehyde 3-phosphate dehydrogenase (GAPDH) promoter, eukaryotic translation initiation factor 4A1 (EIF4A1) promoter, heat shock 70 kDa protein 5 (HSPA5) promoter, heat shock protein 90 kDa beta, member 1 (HSP90B1) promoter, heat shock protein 70 kDa (HSP70) promoter, β-kinesin (β-KIN) promoter, human ROSA26 promoter, ubiquitin C (UBC) promoter, phosphoglycerate kinase-1 (PGK) promoter, cytomegalovirus enhancer/chicken β-actin (CAG) promoter, and β-actin promoter.

In some cases, expression of a switch receptor may be inducible (i.e., controlled by the presence of an inducer). Non-limiting examples of inducible promoters suitable for use include: tetracycline responsive promoter, ecdysone responsive promoter, cumate responsive promoter, glucocorticoid responsive promoter, estrogen responsive promoter, or an RU-486 responsive promoter.

As used herein, “conditional expression” may refer to any type of conditional expression including, but not limited to, inducible expression; repressible expression; expression in cells or tissues having a particular physiological, biological, or disease state, etc. This definition is not intended to exclude cell type or tissue specific expression. Certain embodiments of the invention provide conditional expression of a polynucleotide-of-interest, e.g., expression is controlled by subjecting a cell, tissue, organism, etc., to a treatment or condition that causes the polynucleotide to be expressed or that causes an increase or decrease in expression of the polynucleotide encoded by the polynucleotide-of-interest.

Illustrative examples of inducible promoters/systems include, but are not limited to, steroid-inducible promoters such as promoters for genes encoding glucocorticoid or estrogen receptors (inducible by treatment with the corresponding hormone), metallothionine promoter (inducible by treatment with various heavy metals), MX-1 promoter (inducible by interferon), the “GeneSwitch” mifepristone-regulatable system (Sirin et al., 2003, Gene, 323:67), the cumate inducible gene switch (WO 2002/088346), tetracycline-dependent regulatory systems, etc.

Illustrative examples of promoters suitable for use in particular embodiments include, but are not limited to neuron specific promoters.

In particular embodiments, a polynucleotide contemplated herein comprises a neuron specific promoter or a promoter operative in a neuronal cell.

In particular embodiments, a polynucleotide contemplated herein comprises a neuron specific promoter operable in a trigeminal ganglion (TGG) neuron or a dorsal root ganglion (DRG) neuron.

In particular embodiments, a polynucleotide contemplated herein comprises a neuron specific promoter selected from the group consisting of a calcium/calmodulin-dependent protein kinase II promoter, a tubulin alpha I promoter, a neuron-specific enolase promoter, a platelet-derived growth factor beta chain promoter, an hSYN1 promoter, a TRPV1 promoter, a Nav1.7 promoter, a Nav1.8 promoter, a Nav1.9 promoter, and an Advillin promoter.

In one embodiment, the neuron specific promoter operably linked to a polynucleotide encoding a switch receptor is a human synapsin 1 (SYN1) promoter.

In particular embodiments, polynucleotides contemplated herein comprise at least one (typically two) site(s) for recombination mediated by a site specific recombinase. As used herein, the terms “recombinase” or “site specific recombinase” include excisive or integrative proteins, enzymes, co-factors or associated proteins that are involved in recombination reactions involving one or more recombination sites (e.g., two, three, four, five, six, seven, eight, nine, ten or more.), which may be wild-type proteins (see Landy, Current Opinion in Biotechnology 3:699-707 (1993)), or mutants, derivatives (e.g., fusion proteins containing the recombination protein sequences or fragments thereof), fragments, and variants thereof. Illustrative examples of recombinases suitable for use in particular embodiments of the present invention include, but are not limited to: Cre, Int, IHF, Xis, Flp, Fis, Hin, Gin, ΦC31, Cin, Tn3 resolvase, TndX, XerC, XerD, TnpX, Hjc, Gin, SpCCE1, and ParA.

The polynucleotides may comprise one or more recombination sites for any of a wide variety of site specific recombinases. As used herein, the terms “recombination sequence,” “recombination site,” or “site specific recombination site” refer to a particular nucleic acid sequence to which a recombinase recognizes and binds.

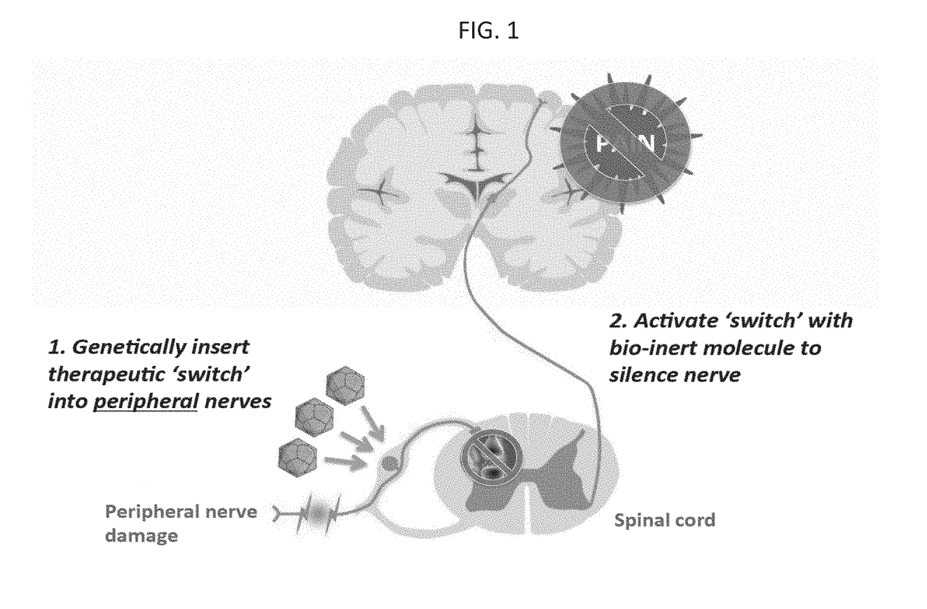

For example, one recombination site for Cre recombinase is loxP which is a 34 base pair sequence comprising two 13 base pair inverted repeats (serving as the recombinase binding sites) flanking an 8 base pair core sequence (see FIG. 1 of Sauer, B., Current Opinion in Biotechnology 5:521-527 (1994)). Other exemplary loxP sites include, but are not limited to: lox511 (Hoess et al., 1996; Bethke and Sauer, 1997), lox5171 (Lee and Saito, 1998), lox2272 (Lee and Saito, 1998), m2 (Langer et al., 2002), lox71 (Albert et al., 1995), and lox66 (Albert et al., 1995).

Suitable recognition sites for the FLP recombinase include, but are not limited to: FRT (McLeod, et al., 1996), F₁, F₂, F₃ (Schlake and Bode, 1994), F₄, F₅ (Schlake and Bode, 1994), FRT(LE) (Senecoff et al., 1988), FRT(RE) (Senecoff et al., 1988).

Other examples of recognition sequences are the attB, attP, attL, and attR sequences, which are recognized by the recombinase enzyme λ Integrase, e.g., phi-c31. The φC31 SSR mediates recombination only between the heterotypic sites attB (34 bp in length) and attP (39 bp in length) (Groth et al., 2000). attB and attP, named for the attachment sites for the phage integrase on the bacterial and phage genomes, respectively, both contain imperfect inverted repeats that are likely bound by φC31 homodimers (Groth et al., 2000). The product sites, attL and attR, are effectively inert to further φC31-mediated recombination (Belteki et al., 2003), making the reaction irreversible. For catalyzing insertions, it has been found that attB-bearing DNA inserts into a genomic attP site more readily than an attP site into a genomic attB site (Thyagarajan et al., 2001; Belteki et al., 2003). Thus, typical strategies position by homologous recombination an attP-bearing “docking site” into a defined locus, which is then partnered with an attB-bearing incoming sequence for insertion.

In particular embodiments, polynucleotides contemplated herein, include one or more polynucleotides-of-interest that encode one or more polypeptides. In particular embodiments, to achieve efficient translation of each of the plurality of polypeptides, the polynucleotide sequences can be separated by one or more IRES sequences or polynucleotide sequences encoding self-cleaving polypeptides.

As used herein, an “internal ribosome entry site” or “IRES” refers to an element that promotes direct internal ribosome entry to the initiation codon, such as ATG, of a cistron (a protein encoding region), thereby leading to the cap-independent translation of the gene. See, e.g., Jackson et al., 1990. Trends Biochem Sci 15(12):477-83) and Jackson and Kaminski. 1995. RNA 1(10):985-1000. Examples of IRES generally employed by those of skill in the art include those described in U.S. Pat. No. 6,692,736. Further examples of “IRES” known in the art include, but are not limited to IRES obtainable from picornavirus (Jackson et al., 1990) and IRES obtainable from viral or cellular mRNA sources, such as for example, immunoglobulin heavy-chain binding protein (BiP), the vascular endothelial growth factor (VEGF) (Huez et al. 1998. Mol. Cell. Biol. 18(11):6178-6190), the fibroblast growth factor 2 (FGF-2), and insulin-like growth factor (IGFII), the translational initiation factor eIF4G and yeast transcription factors TFIID and HAP4, the encephelomycarditis virus (EMCV) which is commercially available from Novagen (Duke et al., 1992. J. Virol 66(3):1602-9) and the VEGF IRES (Huez et al., 1998. Mol Cell Biol 18(11):6178-90). IRES have also been reported in viral genomes of Picornaviridae, Dicistroviridae and Flaviviridae species and in HCV, Friend murine leukemia virus (FrMLV) and Moloney murine leukemia virus (MoMLV).

In one embodiment, the IRES used in polynucleotides contemplated herein is an EMCV IRES.

In particular embodiments, a polynucleotide encoding a polypeptide comprises a consensus Kozak sequence. As used herein, the term “Kozak sequence” refers to a short nucleotide sequence that greatly facilitates the initial binding of mRNA to the small subunit of the ribosome and increases translation. The consensus Kozak sequence is (GCC)RCCATGG (SEQ ID NO: 2), where R is a purine (A or G) (Kozak, 1986. Cell. 44(2):283-92, and Kozak, 1987. Nucleic Acids Res. 15(20):8125-48).

In particular embodiments, polynucleotides comprise a polyadenylation sequence 3′ of a polynucleotide encoding a polypeptide to be expressed. Polyadenylation sequences can promote mRNA stability by addition of a polyA tail to the 3′ end of the coding sequence and thus, contribute to increased translational efficiency. Cleavage and polyadenylation is directed by a poly(A) sequence in the RNA. The core poly(A) sequence for mammalian pre-mRNAs has two recognition elements flanking a cleavage-polyadenylation site. Typically, an almost invariant AAUAAA hexamer lies 20-50 nucleotides upstream of a more variable element rich in U or GU residues. Cleavage of the nascent transcript occurs between these two elements and is coupled to the addition of up to 250 adenosines to the 5′ cleavage product. In particular embodiments, the core poly(A) sequence is an ideal polyA sequence (e.g., AATAAA, ATTAAA, AGTAAA). In particular embodiments the poly(A) sequence is an SV40 polyA sequence, a bovine growth hormone polyA sequence (BGHpA), a rabbit β-globin polyA sequence (rβgpA), or another suitable heterologous or endogenous polyA sequence known in the art.

### G. Polypeptides

The present invention contemplates, in part, compositions comprising switch receptor polypeptides including, but not limited to GPCR, RASSL, DREADD, and LGIC polypeptides and subunits and muteins thereof, polypeptides, fusion polypeptides, and vectors that express polynucleotides encoding the polypeptides.

“Polypeptide,” “polypeptide fragment,” “peptide” and “protein” are used interchangeably, unless specified to the contrary, and according to conventional meaning, i.e., as a sequence or a polymer of amino acids of any length. In one embodiment, a “polypeptide” includes fusion polypeptides and other variants. Polypeptides can be prepared using any of a variety of well-known recombinant and/or synthetic techniques. Polypeptides are not limited to a specific length, e.g., they may comprise a full length protein sequence, a fragment of a full length protein, or a fusion protein, and may include post-translational modifications of the polypeptide, for example, glycosylations, acetylations, phosphorylations and the like, as well as other modifications known in the art, both naturally occurring and non-naturally occurring. A polypeptide can be any protein, peptide, protein fragment or component thereof. A polypeptide can be a protein naturally occurring in nature or a protein that is ordinarily not found in nature. A polypeptide can consist largely of the standard twenty protein-building amino acids or it can be modified to incorporate non-standard amino acids. A polypeptide can be modified, typically by the host cell, by e.g., adding any number of biochemical functional groups, including phosphorylation, acetylation, acylation, formylation, alkylation, methylation, lipid addition (e.g. palmitoylation, myristoylation, prenylation, etc) and carbohydrate addition (e.g. N-linked and O-linked glycosylation, etc). Polypeptides can undergo structural changes in the host cell such as the formation of disulfide bridges or proteolytic cleavage.

An “isolated peptide” or an “isolated polypeptide” and the like, as used herein, refer to in vitro isolation, purification, recombinant production, or synthesis of a peptide or polypeptide molecule from a cellular environment, and from association with other components of the cell, i.e., it is not significantly associated with in vivo substances.

Polypeptides include biologically active “polypeptide fragments.” As used herein, the term “biologically active fragment” or “minimal biologically active fragment” refers to a polypeptide fragment that retains at least 100%, at least 90%, at least 80%, at least 70%, at least 60%, at least 50%, at least 40%, at least 30%, at least 20%, at least 10%, or at least 5% of the naturally occurring polypeptide activity. Polypeptide fragments refer to a polypeptide, which can be monomeric or multimeric, that has an amino-terminal deletion, a carboxyl-terminal deletion, and/or an internal deletion or substitution of one or more amino acids of a naturally-occurring or recombinantly-produced polypeptide. In certain embodiments, a polypeptide fragment can comprise an amino acid chain at least 5 to about 1700 amino acids long. It will be appreciated that in certain embodiments, fragments are at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 150, 200, 250, 300, 350, 400, 450, 500, 550, 600, 650, 700, 750, 800, 850, 900, 950, 1000, 1100, 1200, 1300, 1400, 1500, 1600, 1700 or more amino acids long.

Polypeptides include “polypeptide variants.” Polypeptide variants may differ from a naturally occurring polypeptide in one or more amino acid substitutions, deletions, additions and/or insertions. Such variants may be naturally occurring or may be synthetically generated (engineered), for example, by modifying one or more amino acids of a switch receptor polypeptide sequences. For example, in particular embodiments, it may be desirable to improve the biological properties of a switch receptor polypeptide or the binding specificity of the switch receptor to a heterologous and/or synthetic ligand by introducing one or more substitutions, deletions, additions and/or insertions into the polypeptide. Preferably, polypeptide variants include polypeptides having at least about 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity to switch receptor polypeptides contemplated herein.

As noted above, polypeptides contemplated herein may be altered in various ways including amino acid substitutions, deletions, truncations, and insertions. In particular embodiments, one or more amino acids of a switch polypeptide are altered to confer a unique ligand binding property to the switch receptor. Methods for such manipulations are generally known in the art. For example, amino acid sequence variants of a reference polypeptide can be prepared by mutations in the DNA. Methods for mutagenesis and nucleotide sequence alterations are well known in the art. See, for example, Kunkel (1985, Proc. Natl. Acad. Sci. USA. 82: 488-492), Kunkel et al., (1987, Methods in Enzymol, 154: 367-382), U.S. Pat. No. 4,873,192, Watson, J. D. et al., (Molecular Biology of the Gene, Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found., Washington, D.C.).

In certain embodiments, a variant will contain one or more conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Modifications may be made in the structure of the polynucleotides and polypeptides of the present invention and still obtain a functional molecule that encodes a variant or derivative polypeptide with desirable characteristics. When it is desired to alter the amino acid sequence of a polypeptide to create an equivalent, or even an improved, variant polypeptide of the invention, one skilled in the art, for example, can change one or more of the codons of the encoding DNA sequence, e.g., according to Table 3.

TABLE 3
Amino Acid Codons

| | One | Three | | | | | | |
|---|---|---|---|---|---|---|---|---|
| | letter | letter | | | | | | |
| Amino Acids | code | code | Codons | | | | | |
| Alanine | A | Ala | GCA | GCC | GCG | GCU | | |
| Cysteine | C | Cys | UGC | UGU | | | | |
| Aspartic acid | D | Asp | GAC | GAU | | | | |
| Glutamic acid | E | Glu | GAA | GAG | | | | |
| Phenylalanine | F | Phe | UUC | UUU | | | | |
| Glycine | G | Gly | GGA | GGC | GGG | GGU | | |
| Histidine | H | His | CAC | CAU | | | | |
| Isoleucine | I | Iso | AUA | AUC | AUU | | | |
| Lysine | K | Lys | AAA | AAG | | | | |
| Leucine | L | Leu | UUA | UUG | CUA | CUC | CUG | CUU |
| Methionine | M | Met | AUG | | | | | |
| Asparagine | N | Asn | AAC | AAU | | | | |
| Proline | P | Pro | CCA | CCC | CCG | CCU | | |
| Glutamine | Q | Gln | CAA | CAG | | | | |
| Arginine | R | Arg | AGA | AGG | CGA | CGC | CGG | CGU |
| Serine | S | Ser | AGC | AGU | UCA | UCC | UCG | UCU |
| Threonine | T | Thr | ACA | ACC | ACG | ACU | | |
| Valine | V | Val | GUA | GUC | GUG | GUU | | |
| Tryptophan | W | Trp | UGG | | | | | |
| Tyrosine | Y | Tyr | UAC | UAU | | | | |

Guidance in determining which amino acid residues can be substituted, inserted, or deleted without abolishing biological activity can be found using computer programs well known in the art, such as DNASTAR™ software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224).

In making such changes, the hydropathic index of amino acids may be considered. The importance of the hydropathic amino acid index in conferring interactive biologic function on a protein is generally understood in the art (Kyte and Doolittle, 1982, incorporated herein by reference). Each amino acid has been assigned a hydropathic index on the basis of its hydrophobicity and charge characteristics (Kyte and Doolittle, 1982). These values are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like.

Polypeptide variants further include glycosylated forms, aggregative conjugates with other molecules, and covalent conjugates with unrelated chemical moieties (e.g., pegylated molecules). Covalent variants can be prepared by linking functionalities to groups which are found in the amino acid chain or at the N- or C-terminal residue, as is known in the art. Variants also include allelic variants, species variants, and muteins. Truncations or deletions of regions which do not affect functional activity of the proteins are also variants.

Polypeptides of the present invention include fusion polypeptides. In particular embodiments, fusion polypeptides and polynucleotides encoding fusion polypeptides are provided. Fusion polypeptides and fusion proteins refer to a polypeptide having at least two, three, four, five, six, seven, eight, nine, or ten polypeptide segments.

Fusion polypeptides can comprise one or more polypeptide domains or segments including, but are not limited to cell permeable peptide domains (CPP), Zn-finger DNA binding domains, nuclease domains, chromatin remodeling domains, histone modifying domains, and epigenetic modifying domains, epitope tags (e.g., maltose binding protein (“MBP”), glutathione S transferase (GST), HIS6, MYC, FLAG, V5, VSV-G, and HA), polypeptide linkers, and polypeptide cleavage signals. Fusion polypeptides are typically linked C-terminus to N-terminus, although they can also be linked C-terminus to C-terminus, N-terminus to N-terminus, or N-terminus to C-terminus. The polypeptides of the fusion protein can be in any order. Fusion polypeptides or fusion proteins can also include conservatively modified variants, polymorphic variants, alleles, mutants, subsequences, and interspecies homologs, so long as the desired transcriptional activity of the fusion polypeptide is preserved. Fusion polypeptides may be produced by chemical synthetic methods or by chemical linkage between the two moieties or may generally be prepared using other standard techniques. Ligated DNA sequences comprising the fusion polypeptide are operably linked to suitable transcriptional or translational control elements as discussed elsewhere herein.

Fusion polypeptides may optionally comprise a linker that can be used to link the one or more polypeptides. A peptide linker sequence may be employed to separate any two or more polypeptide components by a distance sufficient to ensure that each polypeptide folds into its appropriate secondary and tertiary structures so as to allow the polypeptide domains to exert their desired functions. Such a peptide linker sequence is incorporated into the fusion polypeptide using standard techniques in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. No. 4,935,233 and U.S. Pat. No. 4,751,180. Linker sequences are not required when a particular fusion polypeptide segment contains non-essential N-terminal amino acid regions that can be used to separate the functional domains and prevent steric interference. Preferred linkers are typically flexible amino acid subsequences which are synthesized as part of a recombinant fusion protein. Linker polypeptides can be between 1 and 200 amino acids in length, between 1 and 100 amino acids in length, or between 1 and 50 amino acids in length, including all integer values in between.

Exemplary linkers include, but are not limited to the following amino acid sequences: DGGGS (SEQ ID NO: 3); TGEKP (SEQ ID NO: 4) (see, e.g., Liu et al., PNAS 5525-5530 (1997)); GGRR (SEQ ID NO: 5) (Pomerantz et al. 1995, supra); (GGGGS)ₙ (SEQ ID NO: 6) (Kim et al., PNAS 93, 1156-1160 (1996.); EGKSSGSGSESKVD (SEQ ID NO: 7) (Chaudhary et al., 1990, Proc. Natl. Acad. Sci. U.S.A. 87:1066-1070); KESGSVSSEQLAQFRSLD (SEQ ID NO: 8) (Bird et al., 1988, Science 242:423-426), GGRRGGGS (SEQ ID NO: 9); LRQRDGERP (SEQ ID NO: 10); LRQKDGGGSERP (SEQ ID NO: 11); LRQKd(GGGS)₂ERP (SEQ ID NO: 12). Alternatively, flexible linkers can be rationally designed using a computer program capable of modeling both DNA-binding sites and the peptides themselves (Desjarlais & Berg, PNAS 90:2256-2260 (1993), PNAS 91:11099-11103 (1994) or by phage display methods.

Fusion polypeptides may further comprise a polypeptide cleavage signal between each of the polypeptide domains described herein. In addition, polypeptide site can be put into any linker peptide sequence. Exemplary polypeptide cleavage signals include polypeptide cleavage recognition sites such as protease cleavage sites, nuclease cleavage sites (e.g., rare restriction enzyme recognition sites, self-cleaving ribozyme recognition sites), and self-cleaving viral oligopeptides (see deFelipe and Ryan, 2004. Traffic, 5(8); 616-26).

Suitable protease cleavages sites and self-cleaving peptides are known to the skilled person (see, e.g., in Ryan et al., 1997. J. Gener. Virol. 78, 699-722; Scymczak et al. (2004) Nature Biotech. 5, 589-594). Exemplary protease cleavage sites include, but are not limited to the cleavage sites of potyvirus NIa proteases (e.g., tobacco etch virus protease), potyvirus HC proteases, potyvirus P1 (P35) proteases, byovirus NIa proteases, byovirus RNA-2-encoded proteases, aphthovirus L proteases, enterovirus 2A proteases, rhinovirus 2A proteases, picorna 3C proteases, comovirus 24K proteases, nepovirus 24K proteases, RTSV (rice tungro spherical virus) 3C-like protease, PYVF (parsnip yellow fleck virus) 3C-like protease, heparin, thrombin, factor Xa and enterokinase. Due to its high cleavage stringency, TEV (tobacco etch virus) protease cleavage sites are preferred in one embodiment, e.g., EXXYXQ(G/S) (SEQ ID NO: 13), for example, ENLYFQG (SEQ ID NO: 14) and ENLYFQS (SEQ ID NO: 15), wherein X represents any amino acid (cleavage by TEV occurs between Q and G or Q and S).

In certain embodiments, the self-cleaving polypeptide site comprises a 2A or 2A-like site, sequence or domain (Donnelly et al., 2001. J. Gen. Virol. 82:1027-1041). In a particular embodiment, the viral 2A peptide is an aphthovirus 2A peptide, a potyvirus 2A peptide, or a cardiovirus 2A peptide. In one embodiment, the viral 2A peptide is selected from the group consisting of: a foot-and-mouth disease virus (FMDV) 2A peptide, an equine rhinitis A virus (ERAV) 2A peptide, a Thosea asigna virus (TaV) 2A peptide, a porcine teschovirus-1 (PTV-1) 2A peptide, a Theilovirus 2A peptide, and an encephalomyocarditis virus 2A peptide.

### H. Viral Vectors

In some aspects, a nucleic acid molecule encoding a switch receptor is delivered to a subject. In some cases, the nucleic acid molecule encoding the switch receptor is delivered to a subject by a vector. In various embodiments, a vector comprises a one or more polynucleotide sequences contemplated herein. The term “vector” is used herein to refer to a nucleic acid molecule capable of transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. A vector can deliver a target nucleic acid to an organism, a cell or a cellular component. In some cases, the vector is an expression vector. An “expression vector” as used herein refers to a vector, for example, a plasmid, that is capable of promoting expression, as well as replication of a nucleic acid incorporated therein. Typically, the nucleic acid to be expressed is “operably linked” to a promoter and/or enhancer, and is subject to transcription regulatory control by the promoter and/or enhancer. In particular cases, a vector is used to deliver a nucleic acid molecule encoding a switch receptor of the disclosure to a subject.

In particular embodiments, any vector suitable for introducing an expression cassette or polynucleotide encoding a switch receptor into a neuronal cell can be employed. Illustrative examples of suitable vectors include, for example, plasmids (e.g., DNA plasmids or RNA plasmids), transposons, cosmids, bacterial artificial chromosomes, and viral vectors. In some cases, the vector is a circular nucleic acid, for e.g., a plasmid, a BAC, a PAC, a YAC, a cosmid, a fosmid, and the like. In some cases, circular nucleic acid molecules can be utilized to deliver a nucleic acid molecule encoding a switch receptor to a subject. For example, a plasmid DNA molecule encoding a switch receptor can be introduced into a cell of a subject whereby the DNA sequence encoding the switch receptor is transcribed into mRNA and the mRNA “message” is translated into a protein product. The circular nucleic acid vector will generally include regulatory elements that regulate the expression of the target protein. For example, the circular nucleic acid vector may include any number of promoters, enhancers, terminators, splice signals, origins of replication, initiation signals, and the like.

In some cases, the vector can include a replicon. A replicon may be any nucleic acid molecule capable of self-replication. In some cases, the replicon is an RNA replicon derived from a virus. A variety of suitable viruses (e.g. RNA viruses) are available, including, but not limited to, alphavirus, picornavirus, flavivirus, coronavirus, pestivirus, rubivirus, calcivirus, and hepacivirus.

In one embodiment, the vector is a viral vector. In some cases, the viral vector is derived from a replication-deficient virus. Non-limiting examples of viral vectors suitable for delivering a nucleic acid molecule of the disclosure to a subject include those derived from adenovirus, retrovirus (e.g., lentivirus), adeno-associated virus (AAV), and herpes simplex-1 (HSV-1). Illustrative examples of suitable viral vectors include, but are not limited to, retroviral vectors (e.g., lentiviral vectors), herpes virus based vectors and parvovirus based vectors (e.g., adeno-associated virus (AAV) based vectors, AAV-adenoviral chimeric vectors, and adenovirus-based vectors).

The term “parvovirus” as used herein encompasses all parvoviruses, including autonomously-replicating parvoviruses and dependoviruses. The autonomous parvoviruses include members of the genera Parvovirus, Erythrovirus, Densovirus, Iteravirus, and Contravirus. Exemplary autonomous parvoviruses include, but are not limited to, mouse minute virus, bovine parvovirus, canine parvovirus, chicken parvovirus, feline panleukopenia virus, feline parvovirus, goose parvovirus, and B19 virus. Other autonomous parvoviruses are known to those skilled in the art. See, e.g., Fields et al., 1996 Virology, volume 2, chapter 69 (3d ed., Lippincott-Raven Publishers).

The genus Dependovirus contains the adeno-associated viruses (AAV), including but not limited to, AAV type 1, AAV type 2, AAV type 3, AAV type 4, AAV type 5, AAV type 6, avian AAV, bovine AAV, canine AAV, equine AAV, and ovine AAV.

In a preferred embodiment, the vector is an AAV vector. In particular cases, the viral vector is an AAV-6 or AAV9 vector. In some embodiments, the AAV vector comprises SEQ ID NO:1.

The genomic organization of all known AAV serotypes is similar. The genome of AAV is a linear, single-stranded DNA molecule that is less than about 5,000 nucleotides (nt) in length. Inverted terminal repeats (ITRs) flank the unique coding nucleotide sequences for the non-structural replication (Rep) proteins and the structural (VP) proteins. The VP proteins (VP1, -2 and -3) form the capsid and contribute to the tropism of the virus. The terminal 145 nt ITRs are self-complementary and are organized so that an energetically stable intramolecular duplex forming a T-shaped hairpin may be formed. These hairpin structures function as an origin for viral DNA replication, serving as primers for the cellular DNA polymerase complex. Following wild-type (wt) AAV infection in mammalian cells the Rep genes are expressed and function in the replication of the viral genome.

In some cases, the outer protein “capsid” of the viral vector occurs in nature, e.g. AAV-1, AAV-2, AAV-3, AAV-4, AAV-5, AAV-6, AAV-7, AAV-8, AAV-9, AAV-10. In particular cases, the capsid is synthetically engineered (e.g. through directed evolution or rational design) to possess certain unique characteristics not present in nature such as altered tropism, increased transduction efficiency, or immune evasion. An example of a rationally designed capsid is the mutation of one or more surface-exposed tyrosine (Y), serine (S), threonine (T), and lysine (K) residues on the VP3 viral capsid protein. Non-limiting examples of viral vectors whose VP3 capsid proteins have been synthetically engineered and are amenable for use with the compositions and methods provided herein include: AAV1(Y705+731F+T492V), AAV2(Y444+500+730F+T491V), AAV3(Y705+731F), AAV5(Y436+693+719F), AAV6(Y705+731F+T492V), AAV8(Y733F), AAV9(Y731F), and AAV10(Y733F). Non-limiting examples of viral vectors that have been engineered through directed evolution and are amenable for use with the compositions and methods provided herein include AAV-7m8 and AAV-ShH10.

A “recombinant parvoviral or AAV vector” (or “rAAV vector”) herein refers to a vector comprising one or more polynucleotides contemplated herein that are flanked by one or more AAV ITRs. Such rAAV vectors can be replicated and packaged into infectious viral particles when present in an insect host cell that is expressing AAV rep and cap gene products (i.e., AAV Rep and Cap proteins). When an rAAV vector is incorporated into a larger nucleic acid construct (e.g., in a chromosome or in another vector such as a plasmid or baculovirus used for cloning or transfection), then the rAAV vector is typically referred to as a “pro-vector” which can be “rescued” by replication and encapsidation in the presence of AAV packaging functions and necessary helper functions.

In particular embodiments, any AAV ITR may be used in the AAV vectors, including ITRs from AAV1, AAV2, AAV3, AAV4, AAV5, AAV6, AAV7, AAV8, AAV9, AAV10, AAV11, AAV12, AAV13, AAV14, AAV15, and AAV16. In one preferred embodiment, an AAV vector contemplated herein comprises one or more AAV2 ITRs.

rAAV vectors comprising two ITRs have a payload capacity of about 4.4 kB. Self-complementary rAAV vectors contain a third ITR and package two strands of the recombinant portion of the vector leaving only about 2.1 kB for the polynucleotides contemplated herein. In one embodiment, the AAV vector is an scAAV vector.

Extended packaging capacities that are roughly double the packaging capacity of an rAAV (about 9kB) have been achieved using dual rAAV vector strategies. Dual vector strategies useful in producing rAAV contemplated herein include, but are not limited to splicing (trans-splicing), homologous recombination (overlapping), or a combination of the two (hybrid). In the dual AAV trans-splicing strategy, a splice donor (SD) signal is placed at the 3′ end of the 5′-half vector and a splice acceptor (SA) signal is placed at the 5′ end of the 3′-half vector. Upon co-infection of the same cell by the dual AAV vectors and inverted terminal repeat (ITR)-mediated head-to-tail concatemerization of the two halves, trans-splicing results in the production of a mature mRNA and full-size protein (Yan et al., 2000). Trans-splicing has been successfully used to express large genes in muscle and retina (Reich et al., 2003; Lai et al., 2005). Alternatively, the two halves of a large transgene expression cassette contained in dual AAV vectors may contain homologous overlapping sequences (at the 3′ end of the 5′-half vector and at the 5′ end of the 3′-half vector, dual AAV overlapping), which will mediate reconstitution of a single large genome by homologous recombination (Duan et al., 2001). This strategy depends on the recombinogenic properties of the transgene overlapping sequences (Ghosh et al., 2006). A third dual AAV strategy (hybrid) is based on adding a highly recombinogenic region from an exogenous gene (i.e., alkaline phosphatase; Ghosh et al., 2008, Ghosh et al., 2011)) to the trans-splicing vectors. The added region is placed downstream of the SD signal in the 5′-half vector and upstream of the SA signal in the 3′-half vector in order to increase recombination between the dual AAVs.

A “hybrid AAV” or “hybrid rAAV” refers to an rAAV genome packaged with a capsid of a different AAV serotype (and preferably, of a different serotype from the one or more AAV ITRs), and may otherwise be referred to as a pseudotyped rAAV. For example, an rAAV type 1, 2, 3, 4, 5, 6, 7, 8, 9, 10, 11, 12, 13, 14, 15, or 16 genome may be encapsidated within an AAV type 1, 2, 3, 4, 5, 6, 7, 8, 9, 10, 11, 12, 13, 14, 15, or 16 capsid or variants thereof, provided that the AAV capsid and genome (and preferably, the one or more AAV ITRs) are of different serotypes. In certain embodiments, a pseudotyped rAAV particle may be referred to as being of the type “x/y”, where “x” indicates the source of ITRs and “y” indicates the serotype of capsid, for example a 2/5 rAAV particle has ITRs from AAV2 and a capsid from AAV6.

In one illustrative embodiment, an AAV vector comprises one or more AAV ITRs and one or more capsid proteins from an AAV serotype selected from the group consisting of AAV1, AAV1(Y705+731F+T492V), AAV2(Y444+500+730F+T491V), AAV3(Y705+731F), AAV5, AAV5(Y436+693+719F), AAV6, AAV6 (VP3 variant Y705F/Y731F/T492V), AAV-7m8, AAV8, AAV8(Y733F), AAV9, AAV9 (VP3 variant Y731F), AAV10(Y733F), and AAV-ShH10.

In one illustrative embodiment, an AAV vector comprises one or more AAV2 ITRs and one or more capsid proteins from an AAV serotype selected from the group consisting of AAV1, AAV1(Y705+731F+T492V), AAV2(Y444+500+730F+T491V), AAV3(Y705+731F), AAV5, AAV5(Y436+693+719F), AAV6, AAV6 (VP3 variant Y705F/Y731F/T492V), AAV-7m8, AAV8, AAV8(Y733F), AAV9, AAV9 (VP3 variant Y731F), AAV10(Y733F), and AAV-ShH10.

In one illustrative embodiment, an AAV vector comprises one or more AAV2 ITRs and one or more capsid proteins from an AAV serotype selected from the group consisting of AAV1, AAV5, AAV6, AAV6 (VP3 variant Y705F/Y731F/T492V), AAV8, AAV9, and AAV9 (VP3 variant Y731F).

In another illustrative embodiment, an AAV vector comprises one or more AAV2 ITRs and one or more capsid proteins from an AAV serotype selected from the group consisting of AAV6, AAV6 (VP3 variant Y705F/Y731F/T492V), AAV9, and AAV9 (VP3 variant Y731F).

In one illustrative embodiment, an AAV vector comprises one or more AAV2 ITRs and one or more capsid proteins from an AAV serotype selected from the group consisting of AAV9, and AAV9 (VP3 variant Y731F).

In one illustrative embodiment, an AAV vector comprises one or more AAV2 ITRs and one or more capsid proteins from an AAV serotype selected from the group consisting of AAV6 and AAV6 (VP3 variant Y705F/Y731F/T492V).

In one illustrative embodiment, an AAV vector comprises one or more AAV2 ITRs and one or more capsid proteins from an AAV6 serotype.

In one illustrative embodiment, an AAV vector comprises one or more AAV2 ITRs and one or more capsid proteins from an AAV6 (VP3 variant Y705F/Y731F/T492V) serotype.

A “host cell” includes cells transfected, infected, or transduced in vivo, ex vivo, or in vitro with a recombinant vector or a polynucleotide of the invention. Host cells may include virus producing cells and cells infected with viral vectors. In particular embodiments, host cells in vivo are infected with viral vector contemplated herein. In certain embodiments, the term “target cell” is used interchangeably with host cell and refers to infected cells of a desired cell type.

High titer AAV preparations can be produced using techniques known in the art, e.g., as described in U.S. Pat. Nos. 5,658,776; 6,566,118; 6,989,264; and 6,995,006; U.S. 2006/0188484; WO98/22607; WO2005/072364; and WO/1999/011764; and Viral Vectors for Gene Therapy: Methods and Protocols, ed. Machida, Humana Press, 2003; Samulski et al., (1989) J. Virology 63, 3822; Xiao et al., (1998) J. Virology 72, 2224; Inoue et al., (1998) J. Virol. 72, 7024. Methods of producing pseudotyped AAV vectors have also been reported (e.g., WO00/28004), as well as various modifications or formulations of AAV vectors, to reduce their immunogenicity upon in vivo administration (see e.g., WO01/23001; WO00/73316; W004/1 12727; WO05/005610; WO99/06562).

### I. Compositions and Formulations

The present invention further includes various pharmaceutical compositions comprising polynucleotides, vectors, and polypeptides contemplated herein and a pharmaceutically acceptable carrier. These pharmaceutical compositions may be used to treat a neurological disease or disorder (e.g., pain). As used herein “pharmaceutically acceptable carrier” includes any and all solvents, dispersion media, coatings, antibacterial and antifungal agents, isotonic and absorption delaying agents, and the like that are physiologically compatible, including pharmaceutically acceptable cell culture media. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the vectors contemplated herein, use thereof in the pharmaceutical compositions of the invention is also contemplated.

The compositions of the invention may comprise one or more polypeptides, polynucleotides, and vectors comprising same, infected cells, etc., as described herein, formulated in pharmaceutically-acceptable or physiologically-acceptable solutions for administration to a cell or an animal, either alone, or in combination with one or more other modalities of therapy. It will also be understood that, if desired, the compositions of the invention may be administered in combination with other agents as well, such as, e.g., cytokines, e.g., anti-inflammatory cytokines, growth factors, hormones, small molecules or various pharmaceutically-active agents. There is virtually no limit to other components that may also be included in the compositions, provided that the additional agents do not adversely affect the ability of the composition to deliver the intended gene therapy.

In some cases, a nucleic acid encoding a switch receptor is delivered to a subject by non-viral or vector means. Any method known to those of skill in the art can be used to deliver a nucleic acid molecule of the disclosure to a subject. These methods include, without limitation, lipofection, nanoparticle delivery, particle bombardment, electroporation, sonication and microinjection.

In some aspects, the compositions include a ligand for e.g., activating a switch receptor of the disclosure. In some aspects, the solid formulation may include a nucleic acid molecule (e.g., vector) encoding a switch receptor (e.g., a GPCR or LGIC). In some aspects, the composition is a solid formulation, particularly useful for e.g., oral administration to a subject in need thereof. In some cases, the vector or ligand may be present in the composition at an amount, for example, of about 0.1 μg, 0.2 μg, 0.3 μg, 0.4 μg, 0.5 μg, 0.6 μg, 0.7 μg, 0.8 μg, 0.9 μg, 1 μg, about 2 μg, about 3 μg, about 4 μg, about 5 μg, about 6 μg, about 7 μg, about 8 μg, about 9 μg, about 10 μg, about 20 μg, about 30 μg, about 40 μg, about 50 μg, about 60 μg, about 70 μg, about 80 μg, about 90 μg, about 100 μg, about 120 μg, about 140 μg, about 160 μg, about 180 μg, about 200 μg, about 220 μg, about 240 μg, about 260 μg, about 280 μg, about 300 μg, about 320 μg, about 340 μg, about 360 μg, about 380 μg, about 400 μg, about 420 μg, about 440 μg, about 460 μg, about 480 μg, about 500 μg, about 520 μg, about 540 μg, about 560 μg, about 580 μg, about 600 μg, about 620 μg, about 640 μg, about 660 μg, about 680 μg, about 700 μg, about 720 μg, about 740 μg, about 760 μg, about 780 μg, about 800 μg, about 820 μg, about 840 μg, about 860 μg, about 880 μg, about 900 μg, about 920 μg, about 940 μg, about 960 μg, about 980 μg, about 1 mg, about 2 mg, about 3 mg, about 4 mg, about 5 mg, about 6 mg, about 7 mg, about 8 mg, about 9 mg, about 10 mg, about 20 mg, about 30 mg, about 40 mg, about 50 mg, about 60 mg, about 70 mg, about 80 mg, about 90 mg, about 100 mg, about 120 mg, about 140 mg, about 160 mg, about 180 mg, about 200 mg, about 220 mg, about 240 mg, about 260 mg, about 280 mg, about 300 mg, about 320 mg, about 340 mg, about 360 mg, about 380 mg, about 400 mg, about 420 mg, about 440 mg, about 460 mg, about 480 mg, about 500 mg, about 520 mg, about 540 mg, about 560 mg, about 580 mg, about 600 mg, about 620 mg, about 640 mg, about 660 mg, about 680 mg, about 700 mg, about 720 mg, about 740 mg, about 760 mg, about 780 mg, about 800 mg, about 820 mg, about 840 mg, about 860 mg, about 880 mg, about 900 mg, about 920 mg, about 940 mg, about 960 mg, about 980 mg, about 1000 mg, or greater than 1000 mg.

Compositions as described herein may include a liquid formulation, a solid formulation, or a combination thereof. Non-limiting examples of formulations may include a tablet, a capsule, a gel, a paste, a liquid solution, a patch, a lollipop, a cream or an aerosol (i.e., a spray). In some instances, the therapeutic agent or drug may be in a crystallized form. Solid formulations may be suitable for oral administration of the composition to a subject in need thereof. In some cases, slow release formulations for oral administration may be prepared in order to achieve a controlled release of the active agent in contact with the body fluids in the gastrointestinal tract, and to provide a substantial constant and effective level of the active agent in the blood plasma. The crystal form may be embedded for this purpose in a polymer matrix of a biological degradable polymer, a water-soluble polymer or a mixture of both, and optionally suitable surfactants. Embedding can mean in this context the incorporation of micro-particles in a matrix of polymers. Controlled release formulations are also obtained through encapsulation of dispersed micro-particles or emulsified micro-droplets via known dispersion or emulsion coating technologies.

The compositions of the present disclosure may further include any number of excipients. Excipients may include any and all solvents, coatings, flavorings, colorings, lubricants, disintegrants, preservatives, sweeteners, binders, diluents, and vehicles (or carriers). Generally, the excipient is compatible with the therapeutic compositions of the present disclosure.

In the pharmaceutical compositions contemplated herein, formulation of pharmaceutically-acceptable excipients and carrier solutions is well-known to those of skill in the art, as is the development of suitable dosing and treatment regimens for using the particular compositions described herein in a variety of treatment regimens, including e.g., oral, parenteral, intravenous, intranasal, intramuscular, intrathecal, intraneural, intraganglion, and intraventricular administration and formulation.

In certain circumstances it will be desirable to deliver the compositions disclosed herein parenterally, intravenously, intramuscularly, intraperitoneally, intrathecally, intraneurally, intraganglionicly, or intraventricularly. Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form should be sterile and should be fluid to the extent that easy syringability exists. It should be stable under the conditions of manufacture and storage and should be preserved against the contaminating action of microorganisms, such as bacteria and fungi. The carrier can be a solvent or dispersion medium containing, for example, water, ethanol, polyol (e.g., glycerol, propylene glycol, mannitol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be facilitated by various antibacterial and antifungal agents, for example, parabenes, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intraperitoneal intrathecal, intraneural, intraganglion, and intraventricular administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion (see, e.g., Remington: The Science and Practice of Pharmacy, 20th Edition. Baltimore, Md.: Lippincott Williams & Wilkins, 2000). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Sterile injectable solutions can be prepared by incorporating the active components in the required amount in the appropriate solvent with the various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

In certain embodiments, the compositions may be delivered by intranasal sprays, inhalation, and/or other aerosol delivery vehicles. Methods for delivering genes, polynucleotides, and peptide compositions directly to the lungs via nasal aerosol sprays has been described e.g., in U.S. Pat. No. 5,756,353 and U.S. Pat. No. 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

In certain embodiments, the delivery may occur by use of liposomes, nanocapsules, microparticles, microspheres, lipid particles, vesicles, optionally mixing with CPP polypeptides, and the like, for the introduction of the compositions of the present invention into suitable host cells. In particular, the compositions of the present invention may be formulated for delivery either encapsulated in a lipid particle, a liposome, a vesicle, a nanosphere, a nanoparticle or the like. The formulation and use of such delivery vehicles can be carried out using known and conventional techniques. The formulations and compositions of the invention may comprise one or more repressors and/or activators comprised of a combination of any number of polypeptides, polynucleotides, and small molecules, as described herein, formulated in pharmaceutically-acceptable or physiologically-acceptable solutions (e.g., culture medium) for administration to a cell or an animal, either alone, or in combination with one or more other modalities of therapy. It will also be understood that, if desired, the compositions of the invention may be administered in combination with other agents as well, such as, e.g., cells, other proteins or polypeptides or various pharmaceutically-active agents.

In a particular embodiment, a formulation or composition according to the present invention comprises a cell contacted with a combination of any number of polypeptides, polynucleotides, and viral vectors, as contemplated herein.

In certain aspects, the present invention provides formulations or compositions suitable for the delivery of viral vectors, e.g, rAAV.

Exemplary formulations for ex vivo delivery may also include the use of various transfection agents known in the art, such as calcium phosphate, electroporation, heat shock and various liposome formulations (i.e., lipid-mediated transfection). Liposomes, as described in greater detail below, are lipid bilayers entrapping a fraction of aqueous fluid. DNA spontaneously associates to the external surface of cationic liposomes (by virtue of its charge) and these liposomes will interact with the cell membrane.

In certain aspects, the present invention provides pharmaceutically acceptable compositions which comprise a therapeutically-effective amount of one or more polynucleotides or polypeptides, as described herein, formulated together with one or more pharmaceutically acceptable carriers (additives) and/or diluents (e.g., pharmaceutically acceptable cell culture medium).

Particular embodiments of the invention may comprise other formulations, such as those that are well known in the pharmaceutical art, and are described, for example, in Remington: The Science and Practice of Pharmacy, 20th Edition. Baltimore, Md.: Lippincott Williams & Wilkins, 2000.

### J. Methods and Indications

The compositions and methods disclosed herein can be utilized to treat a neurological disease or disorder. In some aspects, vectors or compositions disclosed herein are used in the manufacture of a medicament for treating a neurological disease or disorder.

In some cases, the methods and compositions of the disclosure are utilized to treat epilepsy. Compositions described herein may be used to prevent or control epileptic seizures. Epileptic seizures may be classified as tonic-clonic, tonic, clonic, myoclonic, absence or atonic seizures. In some cases, the compositions and methods herein may prevent or reduce the number of epileptic seizures experienced by a subject by about 5%, about 10%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or 100%.

In some cases, the methods and compositions of the disclosure are utilized to treat an eating disorder. An eating disorder may be a mental disorder defined by abnormal eating behaviors that negatively affect a subject's physical or mental health. In some cases, the eating disorder is anorexia nervosa. In other cases, the eating disorder is bulimia nervosa. In some cases, the eating disorder is pica, rumination disorder, avoidant/restrictive food intake disorder, binge eating disorder (BED), other specified feeding and eating disorder (OSFED), compulsive overeating, diabulimia, orthorexia nervosa, selective eating disorder, drunkorexia, pregorexia, or Gourmand syndrome. In some cases, the composition includes a G-protein coupled receptor that increases or decreases the production of one or more molecules associated with an eating disorder. In other cases, the composition includes a ligand-gated ion channel that alters the production of one or more molecules associated with an eating disorder. The one or more molecules associated with an eating disorder may include, without limitation, a molecule of the hypothalamus-pituitary-adrenal (HPA) axis, including vasopressin, corticotropin-releasing hormone (CRH), adrenocorticotropic hormone (ACTH), cortisol, epinephrine, or norepinephrine; as well as serotonin, dopamine, neuropeptide Y, leptin, or ghrelin.

In some cases, the compositions and methods are utilized to treat post-traumatic stress disorder (PTSD), gastroesophageal reflex disease (GERD), addiction (e.g., alcohol, drugs), anxiety, depression, memory loss, dementia, sleep apnea, stroke, urinary incontinence, narcolepsy, essential tremor, movement disorder, atrial fibrillation, cancer (e.g., brain tumors), Parkinson's disease, or Alzheimer's disease. Other non-limiting examples of neurological diseases or disorders that can be treated by the compositions and methods herein include: Abulia, Agraphia, Alcoholism, Alexia, Aneurysm, Amaurosis fugax, Amnesia, Amyotrophic lateral sclerosis (ALS), Angelman syndrome, Aphasia, Apraxia, Arachnoiditis, Arnold-Chiari malformation, Asperger syndrome, Ataxia, Ataxia-telangiectasia, Attention deficit hyperactivity disorder, Auditory processing disorder, Autism spectrum, Bipolar disorder, Bell's palsy, Brachial plexus injury, Brain damage, Brain injury, Brain tumor, Canavan disease, Capgras delusion, Carpal tunnel syndrome, Causalgia, Central pain syndrome, Central pontine myelinolysis, Centronuclear myopathy, Cephalic disorder, Cerebral aneurysm, Cerebral arteriosclerosis, Cerebral atrophy, Cerebral autosomal dominant arteriopathy with subcortical infarcts and leukoencephalopathy (CADASIL), Cerebral gigantism, Cerebral palsy, Cerebral vasculitis, Cervical spinal stenosis, Charcot-Marie-Tooth disease, Chiari malformation, Chorea, Chronic fatigue syndrome, Chronic inflammatory demyelinating polyneuropathy (CIDP), Chronic pain, Coffin-Lowry syndrome, Coma, Complex regional pain syndrome, Compression neuropathy, Congenital facial diplegia, Corticobasal degeneration, Cranial arteritis, Craniosynostosis, Creutzfeldt-Jakob disease, Cumulative trauma disorders, Cushing's syndrome, Cyclothymic disorder, Cytomegalic inclusion body disease (CIBD), Cytomegalovirus Infection, Dandy-Walker syndrome, Dawson disease, De Morsier's syndrome, Dejerine-Klumpke palsy, Dejerine-Sottas disease, Delayed sleep phase syndrome, Dementia, Dermatomyositis, Developmental coordination disorder, Diabetic neuropathy, Diffuse sclerosis, Diplopia, Down syndrome, Dravet syndrome, Duchenne muscular dystrophy, Dysarthria, Dysautonomia, Dyscalculia, Dysgraphia, Dyskinesia, Dyslexia, Dystonia, Empty sella syndrome, Encephalitis, Encephalocele, Encephalotrigeminal angiomatosis, Encopresis, Enuresis, Epilepsy, Epilepsy-intellectual disability in females, Erb's palsy, Erythromelalgia, Exploding head syndrome, Fabry's disease, Fahr's syndrome, Fainting, Familial spastic paralysis, Febrile seizures, Fisher syndrome, Friedreich's ataxia, Fibromyalgia, Foville's syndrome, Fetal alcohol syndrome, Fragile X syndrome, Fragile X-associated tremor/ataxia syndrome (FXTAS), Gaucher's disease, Generalized epilepsy with febrile seizures plus, Gerstmann's syndrome, Giant cell arteritis, Giant cell inclusion disease, Globoid Cell Leukodystrophy, Gray matter heterotopia, Guillain-Barré syndrome, Generalized anxiety disorder, HTLV-1 associated myelopathy, Hallervorden-Spatz disease, Head injury, Headache, Hemifacial Spasm, Hereditary Spastic Paraplegia, Heredopathia atactica polyneuritiformis, Herpes zoster oticus, Herpes zoster, Hirayama syndrome, Hirschsprung's disease, Holmes-Adie syndrome, Holoprosencephaly, Huntington's disease, Hydranencephaly, Hydrocephalus, Hypercortisolism, Hypoxia, Immune-Mediated encephalomyelitis, Inclusion body myositis, Incontinentia pigmenti, Infantile Refsum disease, Infantile spasms, Inflammatory myopathy, Intracranial cyst, Intracranial hypertension, Isodicentric 15, Joubert syndrome, Karak syndrome, Kearns-Sayre syndrome, Kinsbourne syndrome, Kleine-Levin Syndrome, Klippel Feil syndrome, Krabbe disease, Lafora disease, Lambert-Eaton myasthenic syndrome, Landau-Kleffner syndrome, Lateral medullary (Wallenberg) syndrome, Learning disabilities, Leigh's disease, Lennox-Gastaut syndrome, Lesch-Nyhan syndrome, Leukodystrophy, Leukoencephalopathy with vanishing white matter, Lewy body dementia, Lissencephaly, Locked-In syndrome, Lumbar disc disease, Lumbar spinal stenosis, Lyme disease—Neurological Sequelae, Machado-Joseph disease (Spinocerebellar ataxia type 3), Macrencephaly, Macropsia, Mal de debarquement, Megalencephalic leukoencephalopathy with subcortical cysts, Megalencephaly, Melkersson-Rosenthal syndrome, Menieres disease, Meningitis, Menkes disease, Metachromatic leukodystrophy, Microcephaly, Micropsia, Migraine, Miller Fisher syndrome, Mini-stroke (transient ischemic attack), Misophonia, Mitochondrial myopathy, Mobius syndrome, Monomelic amyotrophy, Motor skills disorder, Moyamoya disease, Mucopolysaccharidoses, Multi-infarct dementia, Multifocal motor neuropathy, Multiple sclerosis, Multiple system atrophy, Muscular dystrophy, Myalgic encephalomyelitis, Myasthenia gravis, Myelinoclastic diffuse sclerosis, Myoclonic Encephalopathy of infants, Myoclonus, Myopathy, Myotubular myopathy, Myotonia congenita, Narcolepsy, Neuro-Behcet's disease, Neurofibromatosis, Neuroleptic malignant syndrome, Neurological manifestations of AIDS, Neurological sequelae of lupus, Neuromyotonia, Neuronal ceroid lipofuscinosis, Neuronal migration disorders, Neuropathy, Neurosis, Niemann-Pick disease, Non-24-hour sleep-wake disorder, Nonverbal learning disorder, O'Sullivan-McLeod syndrome, Occipital Neuralgia, Occult Spinal Dysraphism Sequence, Ohtahara syndrome, Olivopontocerebellar atrophy, Opsoclonus myoclonus syndrome, Optic neuritis, Orthostatic Hypotension, Otosclerosis, Overuse syndrome, Palinopsia, Paresthesia, Parkinson's disease, Paramyotonia Congenita, Paraneoplastic diseases, Paroxysmal attacks, Parry-Romberg syndrome, PANDAS, Pelizaeus-Merzbacher disease, Periodic Paralyses, Peripheral neuropathy, Pervasive developmental disorders, Photic sneeze reflex, Phytanic acid storage disease, Pick's disease, Pinched nerve, Pituitary tumors, PMG, Polyneuropathy, Polio, Polymicrogyria, Polymyositis, Porencephaly, Post-Polio syndrome, Postherpetic Neuralgia (PHN), Postural Hypotension, Prader-Willi syndrome, Primary Lateral Sclerosis, Prion diseases, Progressive hemifacial atrophy, Progressive multifocal leukoencephalopathy, Progressive Supranuclear Palsy, Prosopagnosia, Pseudotumor cerebri, Quadrantanopia, Quadriplegia, Rabies, Radiculopathy, Ramsay Hunt syndrome type I, Ramsay Hunt syndrome type II, Ramsay Hunt syndrome type III, Rasmussen encephalitis, Reflex neurovascular dystrophy, Refsum disease, REM sleep behavior disorder, Repetitive stress injury, Restless legs syndrome, Retrovirus-associated myelopathy, Rett syndrome, Reye's syndrome, Rhythmic Movement Disorder, Romberg syndrome, Saint Vitus dance, Sandhoff disease, Schilder's disease, Schizencephaly, Sensory processing disorder, Septo-optic dysplasia, Shaken baby syndrome, Shingles, Shy-Drager syndrome, Sjögren's syndrome, Sleep apnea, Sleeping sickness, Snatiation, Sotos syndrome, Spasticity, Spina bifida, Spinal cord injury, Spinal cord tumors, Spinal muscular atrophy, Spinal and bulbar muscular atrophy, Spinocerebellar ataxia, Split-brain, Steele-Richardson-Olszewski syndrome, Stiff-person syndrome, Stroke, Sturge-Weber syndrome, Stuttering, Subacute sclerosing panencephalitis, Subcortical arteriosclerotic encephalopathy, Superficial siderosis, Sydenham's chorea, Syncope, Synesthesia, Syringomyelia, Tarsal tunnel syndrome, Tardive dyskinesia, Tardive dysphrenia, Tarlov cyst, Tay-Sachs disease, Temporal arteritis, Temporal lobe epilepsy, Tetanus, Tethered spinal cord syndrome, Thomsen disease, Thoracic outlet syndrome, Tic Douloureux, Todd's paralysis, Tourette syndrome, Toxic encephalopathy, Transient ischemic attack, Transmissible spongiform encephalopathies, Transverse myelitis, Traumatic brain injury, Tremor, Trichotillomania, Trigeminal neuralgia, Tropical spastic paraparesis, Trypanosomiasis, Tuberous sclerosis, Unverricht-Lundborg disease, Von Hippel-Lindau disease (VHL), Viliuisk Encephalomyelitis (VE), Wallenberg's syndrome, West syndrome, Whiplash, Williams syndrome, Wilson's disease, or Zellweger syndrome.

In some cases, the compositions and methods disclosed herein can be used to treat brain cancer or brain tumors. Non-limiting examples of brain cancers or tumors that may be amenable to treatment with vectors and compositions described herein include: gliomas including anaplastic astrocytoma (grade III glioma), astrocytoma (grade II glioma), brainstem glioma, ependymoma, ganglioglioma, ganglioneuroma, glioblastoma (grade IV glioma), glioma, juvenile pilocytic astrocytoma (JPA), low-grade astrocytoma (LGA), medullablastoma, mixed glioma, oligodendroglioma, optic nerve glioma, pilocytic astrocytoma (grade I glioma), and primitive neuroectodermal (PNET); skull base tumors including acoustic neuroma (vestibular schwannoma), acromegaly, adenoma, chondrosarcoma, chordoma, craniopharyngioma, epidermoid tumor, glomus jugulare tumor, infratentorial meningioma, meningioma, pituitary adenoma, pituitary tumor, Rathke's cleft cyst; metastatic cancer including brain metastasis, metastatic brain tumor; other brain tumors including brain cyst, choroid plexus papilloma, CNS lymphoma, colloid cyst, cystic tumor, dermoid tumor, germinoma, lymphoma, nasal carcinoma, naso-pharyngeal tumor, pineal tumor, pineoblastoma, pineocytoma, supratentorial meningioma, and vascular tumor; spinal cord tumors including astrocytoma, ependymoma, meningioma, and schwannoma.

In some aspects, methods are disclosed for treating a neurological disease or disorder in a subject. In some cases, a method involves administering a biologically inert agent to a subject suffering from a neurological disease or disorder. In some cases, the subject may heterologously express a G protein-coupled receptor. In another case, the subject may heterologously express a ligand-gated ion channel. The methods may further comprise delivering a nucleic acid molecule encoding the GPCR or LGIC to the subject, prior to administering the biologically inert agent. In particular examples, the GPCR or LGIC is delivered to the subject by a viral vector, as described throughout the disclosure. In some cases, the subject is treated for pain. In other cases, the subject is treated for a satiety disorder (i.e., eating disorder). In some cases, the subject is not treated for epilepsy.

In other cases, the methods include delivering to a subject suffering from a neurological disease, a nucleic acid molecule encoding a GPCR or LGIC, wherein the subject heterologously expresses the GPCR or LGIC. The method further includes administering to the subject a drug that activates the GPCR or LGIC thereby treating the neurological disease. In some cases, the drug is administered to the subject at least one week after delivery of the nucleic acid encoding the GPCR or LGIC. In further cases, the drug is administered to the subject daily for at least three consecutive days. In some embodiments, the methods include delivering to a subject suffering from a neurological disease that is not epilepsy, a GPCR that is hM4Di and a biologically inert agent that is clozapine-N-oxide.

In other cases, the methods include administering to a subject that heterologously expresses a GPCR or LGIC, a drug that activates the GPCR or LGIC, wherein the drug is not an endogenous ligand for the GPCR or LGIC. In some cases, the drug is not a kappa-opioid receptor (KOR)-binding drug. In further cases, the neurological disease is not epilepsy. In yet further cases, the GPCR is a GPCR other than a kappa-opioid receptor (KOR).

In yet other cases, the methods include treating a neurological disease by administering to a subject that heterologously expresses a GPCR or LGIC, a drug that activates the GPCR or LGIC. In some cases, the GPCR or LGIC is selectively expressed in a sensory neuron, a dorsal root ganglion or a trigeminal ganglion.

In other cases, the methods include treating a neurological disease by administering to a subject that heterologously expresses a GPCR or LGIC, a drug that activates the GPCR or LGIC, wherein the drug is FDA-approved, but not FDA-approved for the treatment of the neurological disease.

In other cases, the methods include treating a neurological disease by administering to a subject that heterologously expresses a GPCR or LGIC, a drug that activates the GPCR or LGIC, wherein the drug is administered at a dose of 0.001 μg/kg-10 mg/kg.

In some aspects, the invention contemplates intracranial injection of AAV-hSYN-GlyRM into the hippocampus of a subject and treating the subject with ivermectin to reversibly silence neuronal networks, e.g., networks associated with memory (see, Obenhaus et al., Front Mol Neurosci. 2016; 9: 75). In some embodiments, the GlyRM protein comprises the F207A and A288G mutations.

In some cases, the invention encompasses a method of treating Parkinson's disease in a subject, comprising administering to the subject an AAV vector selected from AAV-hSYN-rM3DS, AAV-hSYN-hM3Dq, and AAV-hSYN-KORD; and administering CNO to the subject. In some cases, transplanted dopamine neurons are transduced with the AAV vector (see, Aldrin-Kirk et al., Neuron. 2016, 90(5):955-968).

In some cases, the invention encompasses a method of treating Alzheimer's disease in a subject, comprising administering to the subject an AAV vector selected from AAV-CAG-hM4D and AAV-CAG-hM3D; and administering CNO to the subject. In some embodiments, the AAV vector is injected into subarachnoid space or CA1 (see, Yuan et al., J Neurosci. 2016, 36(2):632-641).

In certain aspects, the invention encompasses a method of treating fear and/or anxiety in a subject, comprising administering to the subject an AAV vector (e.g., AAV-CamKII-hM3Dq); and administering CNO to the subject. In some embodiments, the AAV vector is injected into amygdala (see, Sengupta et al., The Journal of Neuroscience, 2016, 36(2):385-395).

The present invention contemplates, in part, compositions and methods for controlling, managing, preventing, or treating pain in a subject. “Pain” refers to an uncomfortable feeling and/or an unpleasant sensation in the body of a subject. Feelings of pain can range from mild and occasional to severe and constant. Pain can be classified as acute pain or chronic pain. Pain can be nociceptive pain (i.e., pain caused by tissue damage), neuropathic pain or psychogenic pain. In some cases, the pain is caused by or associated with a disease (e.g., cancer, arthritis, diabetes). In other cases, the pain is caused by injury (e.g., sports injury, trauma). Non-limiting examples of pain that are amenable to treatment with the compositions and methods herein include: neuropathic pain including peripheral neuropathy, diabetic neuropathy, post herpetic neuralgia, trigeminal neuralgia, back pain, neuropathy associated with cancer, neuropathy associated with HIV/AIDS, phantom limb pain, carpal tunnel syndrome, central post-stroke pain, pain associated with chronic alcoholism, hypothyroidism, uremia, pain associated with multiple sclerosis, pain associated with spinal cord injury, pain associated with Parkinson's disease, epilepsy, osteoarthritic pain, rheumatoid arthritic pain, visceral pain, and pain associated with vitamin deficiency; and nociceptive pain including pain associated with central nervous system trauma, strains/sprains, and burns; myocardial infarction, acute pancreatitis, post-operative pain, posttraumatic pain, renal colic, pain associated with cancer, pain associated with fibromyalgia, pain associated with carpal tunnel syndrome, and back pain.

The compositions and methods herein may be utilized to ameliorate a level of pain in a subject. In some cases, a level of pain in a subject is ameliorated by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or 100%. A level of pain in a subject can be assessed by a variety of methods. In some cases, a level of pain is assessed by self-reporting (i.e., a human subject expresses a verbal report of the level of pain he/she is experiencing). In some cases, a level of pain is assessed by behavioral indicators of pain, for example, facial expressions, limb movements, vocalization, restlessness and guarding. These types of assessments may be useful for example when a subject is unable to self-report (e.g., an infant, an unconscious subject, a non-human subject). A level of pain may be assessed after treatment with a composition of the disclosure as compared to the level of pain the subject was experiencing prior to treatment with the composition.

In various embodiments, a method for controlling, managing, preventing, or treating pain in a subject comprises administering to the subject an effective amount of a vector contemplated herein. Without wishing to be bound by any particular theory, the present invention contemplates using the vectors disclosed herein to modulate neuronal activity to alleviate pain in the subject.

In various embodiments, a vector encoding a switch receptor that activates or depolarizes neuronal cells is administered to (or introduced into) one or more neuronal cells that decrease pain sensation, e.g., inhibitory interneurons. In the presence of ligand the neuronal cell expressing the switch receptor, is activated and decreases the sensitivity to pain potentiating the analgesic effect of stimulating these neuronal cells.

In various embodiments, a vector encoding a switch receptor that deactivates or hyperpolarizes neuronal cells is administered to (or introduced into) one or more neuronal cells that increase pain sensation or sensitivity to pain, e.g., nociceptor, peripheral sensory neurons, C-fibers, Aδ fibers, Aβ fibers, DRG neurons, TGG neurons, and the like. In the presence of ligand the neuronal cell expressing the switch receptor, is deactivated and decreases the sensitivity to pain and potentiating an analgesic effect.

Targeting expression of a switch receptor to a sub-population of nociceptors can be achieved by one or more of: selection of the vector (e.g., AAV1, AAV1(Y705+731F+T492V), AAV2(Y444+500+730F+T491V), AAV3(Y705+731F), AAV5, AAV5(Y436+693+719F), AAV6, AAV6 (VP3 variant Y705F/Y731F/T492V), AAV-7m8, AAV8, AAV8(Y733F), AAV9, AAV9 (VP3 variant Y731F), AAV10(Y733F), and AAV-ShH10); selection of a promoter; and delivery means.

In particular embodiments, the compositions and methods contemplated herein are effective in reducing pain.

Illustrative examples of pain that are amenable to treatment with the vectors, compositions, and methods contemplated herein, include but are not limited to acute pain, chronic pain, neuropathic pain, nociceptive pain, allodynia, inflammatory pain, inflammatory hyperalgesia, neuropathies, neuralgia, diabetic neuropathy, human immunodeficiency virus-related neuropathy, nerve injury, rheumatoid arthritic pain, osteoarthritic pain, burns, back pain, eye pain, visceral pain, cancer pain (e.g., bone cancer pain), dental pain, headache, migraine, carpal tunnel syndrome, fibromyalgia, neuritis, sciatica, pelvic hypersensitivity, pelvic pain, post herpetic neuralgia, post-operative pain, post stroke pain, and menstrual pain.

Pain can be classified as acute or chronic. “Acute pain” refers to pain that begins suddenly and is usually sharp in quality. Acute pain might be mild and last just a moment, or it might be severe and last for weeks or months. In most cases, acute pain does not last longer than three months, and it disappears when the underlying cause of pain has been treated or has healed. Unrelieved acute pain, however, may lead to chronic pain. “Chronic pain” refers to ongoing or recurrent pain, lasting beyond the usual course of acute illness or injury or lasting for more than three to six months, and which adversely affects the individual's well-being. In particular embodiments, the term “chronic pain” refers to pain that continues when it should not. Chronic pain can be nociceptive pain or neuropathic pain.

In particular embodiments, the compositions and methods contemplated herein are effective in reducing acute pain.

In particular embodiments, the compositions and methods contemplated herein are effective in reducing chronic pain.

Clinical pain is present when discomfort and abnormal sensitivity feature among the patient's symptoms. Individuals can present with various pain symptoms. Such symptoms include: 1) spontaneous pain which may be dull, burning, or stabbing; 2) exaggerated pain responses to noxious stimuli (hyperalgesia); and 3) pain produced by normally innocuous stimuli (allodynia-Meyer et al., 1994, Textbook of Pain, 13-44). Although patients suffering from various forms of acute and chronic pain may have similar symptoms, the underlying mechanisms may be different and may, therefore, require different treatment strategies. Pain can also therefore be divided into a number of different subtypes according to differing pathophysiology, including nociceptive pain, inflammatory pain, and neuropathic pain.

In particular embodiments, the compositions and methods contemplated herein are effective in reducing nociceptive pain.

In particular embodiments, the compositions and methods contemplated herein are effective in reducing inflammatory pain.

In particular embodiments, the compositions and methods contemplated herein are effective in reducing neuropathic pain.

Nociceptive pain is induced by tissue injury or by intense stimuli with the potential to cause injury. Moderate to severe acute nociceptive pain is a prominent feature of pain from central nervous system trauma, strains/sprains, burns, myocardial infarction and acute pancreatitis, post-operative pain (pain following any type of surgical procedure), posttraumatic pain, renal colic, cancer pain and back pain. Cancer pain may be chronic pain such as tumor related pain (e.g., bone pain, headache, facial pain or visceral pain) or pain associated with cancer therapy (e.g., postchemotherapy syndrome, chronic postsurgical pain syndrome or post radiation syndrome). Cancer pain may also occur in response to chemotherapy, immunotherapy, hormonal therapy or radiotherapy. Back pain may be due to herniated or ruptured intervertebral discs or abnormalities of the lumber facet joints, sacroiliac joints, paraspinal muscles or the posterior longitudinal ligament. Back pain may resolve naturally but in some patients, where it lasts over 12 weeks, it becomes a chronic condition which can be particularly debilitating.

Neuropathic pain can be defined as pain initiated or caused by a primary lesion or dysfunction in the nervous system. Etiologies of neuropathic pain include, e.g., peripheral neuropathy, diabetic neuropathy, post herpetic neuralgia, trigeminal neuralgia, back pain, cancer neuropathy, HIV neuropathy, phantom limb pain, carpal tunnel syndrome, central post-stroke pain and pain associated with chronic alcoholism, hypothyroidism, uremia, multiple sclerosis, spinal cord injury, Parkinson's disease, epilepsy, and vitamin deficiency.

Neuropathic pain can be related to a pain disorder, a term referring to a disease, disorder or condition associated with or caused by pain. Illustrative examples of pain disorders include arthritis, allodynia, a typical trigeminal neuralgia, trigeminal neuralgia, somatoform disorder, hypoesthesis, hypealgesia, neuralgia, neuritis, neurogenic pain, analgesia, anesthesia dolorosa, causlagia, sciatic nerve pain disorder, degenerative joint disorder, fibromyalgia, visceral disease, chronic pain disorders, migraine/headache pain, chronic fatigue syndrome, complex regional pain syndrome, neurodystrophy, plantar fasciitis or pain associated with cancer.

The inflammatory process is a complex series of biochemical and cellular events, activated in response to tissue injury or the presence of foreign substances, which results in swelling and pain. Arthritic pain is a common inflammatory pain.

Other types of pain that are amenable to treatment with the vectors, compositions, and methods contemplated herein, include but are not limited to pain resulting from musculoskeletal disorders, including myalgia, fibromyalgia, spondylitis, sero-negative (non-rheumatoid) arthropathies, non-articular rheumatism, dystrophinopathy, glycogenolysis, polymyositis and pyomyositis; heart and vascular pain, including pain caused by angina, myocardical infarction, mitral stenosis, pericarditis, Raynaud's phenomenon, scleredoma and skeletal muscle ischemia; head pain, such as migraine (including migraine with aura and migraine without aura), cluster headache, tension-type headache mixed headache and headache associated with vascular disorders; and orofacial pain, including dental pain, otic pain, burning mouth syndrome, and temporomandibular myofascial pain.

The ability of the compositions and methods contemplated herein to reduce the amount of pain experienced by a human subject can be determined using a variety of pain scales. Patient self-reporting can be used to assess whether pain is reduced; see, e.g., Katz and Melzack (1999) Surg. Clin. North Am. 79:231. Alternatively, an observational pain scale can be used. The LANSS Pain Scale can be used to assess whether pain is reduced; see, e.g., Bennett (2001) Pain 92:147. A visual analog pain scale can be used; see, e.g., Schmader (2002) Clin. J. Pain 18:350. The Likert pain scale can be used; e.g., where 0 is no pain, 5 is moderate pain, and 10 is the worst pain possible. Self-report pain scales for children include, e.g., Faces Pain Scale; Wong-Baker FACES Pain Rating Scale; and Colored Analog Scale. Self-report pain scales for adults include, e.g., Visual Analog Scale; Verbal Numerical Rating Scale; Verbal Descriptor Scale; and Brief Pain Inventory. Pain measurement scales include, e.g., Alder Hey Triage Pain Score (Stewart et al. (2004) Arch. Dis. Child. 89:625); Behavioral Pain Scale (Payen et al. (2001) Critical Care Medicine 29:2258); Brief Pain Inventory (Cleeland and Ryan (1994) Ann. Acad. Med. Singapore 23: 129); Checklist of Nonverbal Pain Indicators (Feldt (2000) Pain Manag. Nurs. 1: 13); Critical-Care Pain Observation Tool (Gelinas et al. (2006) Am. J. Crit. Care 15:420); COMFORT scale (Ambuel et al. (1992) J. Pediatric Psychol. 17:95); Dallas Pain Questionnaire (Ozguler et al. (2002) Spine 27:1783); Dolorimeter Pain Index (Hardy et al. (1952) Pain Sensations and Reactions Baltimore: The Williams & Wilkins Co.); Faces Pain Scale—Revised (Hicks et al. (2001) Pain 93:173); Face Legs Activity Cry Consolability Scale; McGill Pain Questionnaire (Melzack (1975) Pain 1:277); Descriptor Differential Scale (Gracely and Kwilosz (1988) Pain 35:279); Numerical 1 l point Box (Jensen et al. (1989) Clin. J. Pain 5: 153); Numeric Rating Scale (Hartrick et al. (2003) Pain Pract. 3:310); Wong-Baker FACES Pain Rating Scale; and Visual Analog Scale (Huskisson (1982) J. Rheumatol. 9:768).

In particular embodiments, a method comprising the introduction of a vector comprising a switch receptor into a neuronal cell and controlling the activity of the cell by providing a ligand that activates the switch receptor, thereby relieves pain in the subject. The method provides significant analgesia for pain without off-target effects, such as general central nervous system depression. In certain embodiments, the method provides a 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or more reduction in the neuropathic pain in a subject compared to an untreated subject.

In particular embodiments, the vectors contemplated herein are administered or introduced into one or more neuronal cells. The neuronal cells may be the same type of neuronal cells, or a mixed population of different types of neuronal cells.

In one embodiment, the neuronal cell is a nociceptor or peripheral sensory neuron.

Illustrative examples of sensory neurons include, but are not limited to, dorsal root ganglion (DRG) neurons and trigeminal ganglion (TGG) neurons.

In one embodiment, the neuronal cell is an inhibitory interneuron involved in the neuronal pain circuit.

In some cases, a vector encoding a switch receptor is administered to a subject in need thereof. Non-limiting examples of methods of administration include sub cutaneous administration, intravenous administration, intramuscular administration, intradermal administration, intraperitoneal administration, oral administration, infusion, intracranial administration, intrathecal administration, intranasal administration, intraganglionic administration, intraspinal administration, cisterna magna administration and intraneural administration. In some cases, administration can involve injection of a liquid formulation of the vector. In other cases, administration can involve oral delivery of a solid formulation of the vector. In some cases, the oral formulation can be administered with food. In particular embodiments, a vector is parenterally, intravenously, intramuscularly, intraperitoneally, intrathecally, intraneurally, intraganglionicly, intraspinally, or intraventricularly administered to a subject in order to introduce the vector into one or more neuronal cells. In various embodiments, the vector is rAAV.

In one embodiment, AAV is administered to sensory neuron or nociceptor, e.g., DRG neurons, TGG neurons, etc. by intrathecal (IT) or intraganglionic (IG) administration.

The IT route delivers AAV to the cerebrospinal fluid (CSF). This route of administration may be suitable for the treatment of e.g., chronic pain or other peripheral nervous system (PNS) or central nervous system (CNS) indications. In animals, IT administration has been achieved by inserting an IT catheter through the cisterna magna and advancing it caudally to the lumbar level. In humans, IT delivery can be easily performed by lumbar puncture (LP), a routine bedside procedure with excellent safety profile.

In a particular case, a vector may be administered to a subject by intraganglionic administration. Intraganglionic administration may involve an injection directly into one or more ganglia. The IG route may deliver AAV directly into the DRG or TGG parenchyma. In animals, IG administration to the DRG is performed by an open neurosurgical procedure that is not desirable in humans because it would require a complicated and invasive procedure. In humans, a minimally invasive, CT imaging-guided technique to safely target the DRG can be used. A customized needle assembly for convection enhanced delivery (CED) can be used to deliver AAV into the DRG parenchyma. In a non-limiting example, a vector of the disclosure may be delivered to one or more dorsal root ganglia and/or trigeminal ganglia for the treatment of chronic pain. In another non-limiting example, a vector of the disclosure may be delivered to the nodose ganglion (vagus nerve) to treat epilepsy.

In yet another particular case, a vector may be administered to the subject by intracranial administration (i.e., directly into the brain). In non-limiting examples of intracranial administration, a vector of the disclosure may be delivered into the cortex of the brain to treat e.g., an epileptic seizure focus, into the paraventricular hypothalamus to treat e.g., a satiety disorder, or into the amygdala central nucleus to treat e.g., a satiety disorder. In another particular case, a vector may be administered to a subject by intraneural injection (i.e., directly into a nerve). The nerve may be selected based on the indication to be treated, for example, injection into the sciatic nerve to treat chronic pain or injection into the vagal nerve to treat epilepsy or a satiety disorder. In yet another particular case, a vector may be administered to a subject by subcutaneous injection, for example, into the sensory nerve terminals to treat chronic pain.

A vector dose may be expressed as the number of vector genome units delivered to a subject. A “vector genome unit” as used herein refers to the number of individual vector genomes administered in a dose. The size of an individual vector genome will generally depend on the type of viral vector used. Vector genomes of the disclosure may be from about 1.0 kilobase, 1.5 kilobases, 2.0 kilobases, 2.5 kilobases, 3.0 kilobases, 3.5 kilobases, 4.0 kilobases, 4.5 kilobases, 5.0 kilobases, 5.5 kilobases, 6.0 kilobases, 6.5 kilobases, 7.0 kilobases, 7.5 kilobases, 8.0 kilobases, 8.5 kilobases, 9.0 kilobases, 9.5 kilobases, 10.0 kilobases, to more than 10.0 kilobases. Therefore, a single vector genome may include up to or greater than 10,000 base pairs of nucleotides. In some cases, a vector dose may be about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹² 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴ 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, 1×10¹⁶, 2×10¹⁶, 3×10¹⁶, 4×10¹⁶, 5×10¹⁶, 6×10¹⁶, 7×10¹⁶, 8×10¹⁶, 9×10¹⁶ 1×10¹⁷, 2×10¹⁷, 3×10¹⁷, 4×10¹⁷, 5×10¹⁷, 6×10¹⁷, 7×10¹⁷, 8×10¹⁷, 9×10¹⁷, 1×10¹⁸, 2×10¹⁸, 3×10¹⁸, 4×10¹⁸, 5×10¹⁸, 6×10¹⁸, 7×10¹⁸, 8×10¹⁸, 9×10¹⁸, 1×10¹⁹, 2×10¹⁹, 3×10¹⁹, 4×10¹⁹, 5×10¹⁹, 6×10¹⁹, 7×10¹⁹, 8×10¹⁹, 9×10¹⁹, 1×10²⁰, 2×10²⁰, 3×10²⁰, 4×10²⁰, 5×10²⁰, 6×10²⁰, 7×10²⁰, 8×10²⁰, 9×10²⁰ or more vector genome units.

In particular embodiments, a vector contemplated herein is administered to a subject at a titer of at least about 1×10⁹ genome particles/mL, at least about 1×10¹⁰ genome particles/mL, at least about 5×10¹⁰ genome particles/mL, at least about 1×10¹¹ genome particles/mL, at least about 5×10¹¹ genome particles/mL, at least about 1×10¹² genome particles/mL, at least about 5×10¹² genome particles/mL, at least about 6×10¹² genome particles/mL, at least about 7×10¹² genome particles/mL, at least about 8×10¹² genome particles/mL, at least about 9×10¹² genome particles/mL, at least about 10×10¹² genome particles/mL, at least about 15×10¹² genome particles/mL, at least about 20×10¹² genome particles/mL, at least about 25×10¹² genome particles/mL, at least about 50×10¹² genome particles/mL, or at least about 100×10¹² genome particles/mL. The terms “genome particles (gp),” or “genome equivalents,” or “genome copies” (gc) as used in reference to a viral titer, refer to the number of virions containing the recombinant AAV DNA genome, regardless of infectivity or functionality. The number of genome particles in a particular vector preparation can be measured by procedures such as described in the Examples herein, or for example, in Clark et al. (1999) Hum. Gene Ther., 10:1031-1039; Veldwijk et al. (2002) Mol. Ther., 6:272-278

A vector of the disclosure may be administered in a volume of fluid. In some cases, a vector may be administered in a volume of about 0.1 mL, 0.2 mL, 0.3 mL, 0.4 mL, 0.5 mL, 0.6 mL, 0.7 mL, 0.8 mL, 0.9 mL, 1.0 mL, 2.0 mL, 3.0 mL, 4.0 mL, 5.0 mL, 6.0 mL, 7.0 mL, 8.0 mL, 9.0 mL, 10.0 mL, 11.0 mL, 12.0 mL, 13.0 mL, 14.0 mL, 15.0 mL, 16.0 mL, 17.0 mL, 18.0 mL, 19.0 mL, 20.0 mL or greater than 20.0 mL. In some cases, a vector dose may be expressed as a concentration or titer of vector administered to a subject. In this case, a vector dose may be expressed as the number of vector genome units per volume (i.e., genome units/volume).

In particular embodiments, a vector contemplated herein is administered to a subject at a titer of at least about 5×10⁹ infectious units/mL, at least about 6×10⁹ infectious units/mL, at least about 7×10⁹ infectious units/mL, at least about 8×10⁹ infectious units/mL, at least about 9×10⁹ infectious units/mL, at least about 10×10⁹ infectious units/mL, at least about 15×10⁹ infectious units/mL, at least about 20×10⁹ infectious units/mL, at least about 25×10⁹ infectious units/mL, at least about 50×10⁹ infectious units/mL, or at least about 100×10⁹ infectious units/mL. The terms “infection unit (iu),” “infectious particle,” or “replication unit,” as used in reference to a viral titer, refer to the number of infectious and replication-competent recombinant AAV vector particles as measured by the infectious center assay, also known as replication center assay, as described, for example, in McLaughlin et al. (1988) J. Virol., 62:1963-1973.

In particular embodiments, a vector contemplated herein is administered to a subject at a titer of at least about 5×10¹⁰ transducing units/mL, at least about 6×10¹⁰ transducing units/mL, at least about 7×10¹⁰ transducing units/mL, at least about 8×10¹⁰ transducing units/mL, at least about 9×10¹⁰ transducing units/mL, at least about 10×10¹⁰ transducing units/mL, at least about 15×10¹⁰ transducing units/mL, at least about 20×10¹⁰ transducing units/mL, at least about 25×10¹⁰ transducing units/mL, at least about 50×10¹⁰ transducing units/mL, or at least about 100×10¹⁰ transducing units/mL. The term “transducing unit (tu)” as used in reference to a viral titer, refers to the number of infectious recombinant AAV vector particles that result in the production of a functional transgene product as measured in functional assays such as described in Examples herein, or for example, in Xiao et al. (1997) Exp. Neurobiol., 144:113-124; or in Fisher et al. (1996) J. Virol., 70:520-532 (LFU assay).

The vector dose will generally be determined by the route of administration. In a particular example, an intraganglionic injection may include from about 1×10⁹ to about 1×10¹³ vector genomes in a volume from about 0.1 mL to about 1.0 mL. In another particular case, an intrathecal injection may include from about 1×10¹⁰ to about 1×10¹⁵ vector genomes in a volume from about 1.0 mL to about 12.0 mL. In yet another particular case, an intracranial injection may include from about 1×10⁹ to about 1×10¹³ vector genomes in a volume from about 0.1 mL to about 1.0 mL. In another particular case, an intraneural injection may include from about 1×10⁹ to about 1×10¹³ vector genomes in a volume from about 0.1 mL to about 1.0 mL. In another particular example, an intraspinal injection may include from about 1×10⁹ to about 1×10¹³ vector genomes in a volume from about 0.1 mL to about 1.0 mL. In yet another particular case, a cisterna magna infusion may include from about 5×10⁹ to about 5×10¹³ vector genomes in a volume from about 0.5 mL to about 5.0 mL. In yet another particular case, a subcutaneous injection may include from about 1×10⁹ to about 1×10¹³ vector genomes in a volume from about 0.1 mL to about 1.0 mL.

In some cases, a vector is delivered to a subject by infusion. A vector dose delivered to a subject by infusion can be measured as a vector infusion rate. Non-limiting examples of vector infusion rates include: 1-10 μl/min for intraganglionic, intraspinal, intracranial or intraneural administration; and 10-1000 μl/min for intrathecal or cisterna magna administration. In some cases, the vector is delivered to a subject by MRI-guided Convection Enhanced Delivery (CED). This technique enables increased viral spread and transduction distributed throughout large volumes of the brain, as well as reduces reflux of the vector along the needle path.

In various embodiments, a method is provided comprising administering a vector encoding a switch receptor, that deactivates or hyperpolarizes neuronal cells, to one or more neuronal cells that increase pain sensation or sensitivity to pain, and administering a ligand that specifically binds the neuronal cell expressing the switch receptor to the subject, thereby deactivating the cell, decreasing the sensitivity to pain and potentiating an analgesic effect.

In various embodiments, a method is provided comprising administering a vector encoding a switch receptor, that activates or polarizes neuronal cells, to one or more neuronal cells that decrease pain sensation or sensitivity to pain, and administering a ligand that specifically binds the neuronal cell expressing the switch receptor to the subject, thereby activating the cell, decreasing the sensitivity to pain and potentiating an analgesic effect.

Formulations of ligands may be administered to a subject by various routes. Non-limiting examples of methods of administration include subcutaneous administration, intravenous administration, intramuscular administration, transdermal administration, intradermal administration, intraperitoneal administration, oral administration, infusion, intracranial administration, intrathecal administration, intranasal administration, intraganglionic administration, and intraneural administration. In some cases, administration can involve injection of a liquid formulation of the ligand. In other cases, administration can involve oral delivery of a solid formulation of the ligand. In a particular case, a ligand is administered by oral administration (e.g., a pill, tablet, capsule and the like). In some cases, the oral composition can be administered with food. In another particular case, a ligand is administered by intrathecal injection (i.e., into the subarachnoid space of the spinal cord) for delivery to the cerebrospinal fluid (CSF) of the subject. In another particular case, a ligand is administered topically (e.g., dermal patch, cream, lotion, ointment and the like).

The dosages of the ligands administered to a subject are not subject to absolute limits, but will depend on the nature of the composition and its active ingredients and its unwanted side effects (e.g., immune response against the antibody), the subject being treated and the type of condition being treated and the manner of administration. Generally, the dose will be a therapeutically effective amount, such as an amount sufficient to achieve a desired biological effect, for example an amount that is effective to decrease or attenuate the level of pain experienced by the subject. In particular embodiments, the dose can also be a prophylactic amount or an effective amount. A therapeutically effective amount of ligand may depend on the route of administration, the indication being treated, and/or the ligand selected for use.

In one embodiment, the ligand is first administered to the subject prior to administration of the vector. A therapeutically effective amount of ligand may be administered to a subject at some time after delivery of a vector. Generally, after delivery of a vector, there will be a period of time required for one or more cells of the subject to generate a protein (i.e., switch receptor) encoded by the vector. During this period of time, administration of a ligand to the subject may not be beneficial to the subject. In this situation, it may be suitable to administer the ligand after an amount of switch receptor has been produced by one or more cells of the subject.

In one embodiment, the ligand is first administered to the subject at about the same time that the vector is administered to the subject.

In one embodiment, the ligand is first administered 1, 2, 3, 4, 5, 6, 7, 8, 9, 11, or 12 hours, days, weeks, months, or years after administration of the vector to the subject. In some cases, a therapeutically effective amount of a ligand may be administered to a subject at least one day, two days, three days, four days, five days, six days, seven days, eight days, nine days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or more than 30 days after delivery of the vector. In a particular example, a therapeutically effective amount of a ligand is administered to a subject at least one week after delivery of a vector. In a further example, the therapeutically effective amount of ligand is administered to the subject daily for at least three consecutive days.

A therapeutically effective amount or dose of a ligand of the disclosure can be expressed as mg or μg of the ligand per kg of subject body mass. In some instances, a therapeutically effective amount of a ligand may be about 0.001 μg/kg, about 0.005 μg/kg, about 0.01 μg/kg, about 0.05 μg/kg, about 0.1 μg/kg, about 0.5 μg/kg, about 1 μg/kg, about 2 μg/kg, about 3 μg/kg, about 4 μg/kg, about 5 μg/kg, about 6 μg/kg, about 7 μg/kg, about 8 μg/kg, about 9 μg/kg, about 10 μg/kg, about 20 μg/kg, about 30 μg/kg, about 40 μg/kg, about 50 μg/kg, about 60 μg/kg, about 70 μg/kg, about 80 μg/kg, about 90 μg/kg, about 100 μg/kg, about 120 μg/kg, about 140 μg/kg, about 160 μg/kg, about 180 μg/kg, about 200 μg/kg, about 220 μg/kg, about 240 μg/kg, about 260 μg/kg, about 280 μg/kg, about 300 μg/kg, about 320 μg/kg, about 340 μg/kg, about 360 μg/kg, about 380 μg/kg, about 400 μg/kg, about 420 μg/kg, about 440 μg/kg, about 460 μg/kg, about 480 μg/kg, about 500 μg/kg, about 520 μg/kg, about 540 μg/kg, about 560 μg/kg, about 580 μg/kg, about 600 μg/kg, about 620 μg/kg, about 640 μg/kg, about 660 μg/kg, about 680 μg/kg, about 700 μg/kg, about 720 μg/kg, about 740 μg/kg, about 760 μg/kg, about 780 μg/kg, about 800 μg/kg, about 820 μg/kg, about 840 μg/kg, about 860 μg/kg, about 880 μg/kg, about 900 μg/kg, about 920 μg/kg, about 940 μg/kg, about 960 μg/kg, about 980 μg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, or greater than 10 mg/kg.

In particular embodiments, the dose of ligand administered to a subject is at least about 0.001 micrograms per kilogram (μg/kg), at least about 0.005 μg/kg, at least about 0.01 μg/kg, at least about 0.05 μg/kg, at least about 0.1 μg/kg, at least about 0.5 μg/kg, 0.001 milligrams per kilogram (mg/kg), at least about 0.005 mg/kg, at least about 0.01 mg/kg, at least about 0.05 mg/kg, at least about 0.1 mg/kg, at least about 0.5 mg/kg, at least about 1 mg/kg, at least about 2 mg/kg, at least about 3 mg/kg, at least about 4 mg/kg, at least about 5 mg/kg, at least about 5 mg/kg, at least about 6 mg/kg, at least about 7 mg/kg, at least about 8 mg/kg, at least about 8 mg/kg, at least about 9 mg/kg, or at least about 10 or more mg/kg.

In particular embodiments, the dose of ligand administered to a subject is at least about 0.001 μg/kg to at least about 10 mg/kg, at least about 0.01 μg/kg to at least about 10 mg/kg, at least about 0.1 μg/kg to at least about 10 mg/kg, at least about 1 μg/kg to at least about 10 mg/kg, at least about 0.01 mg/kg to at least about 10 mg/kg, at least about 0.1 mg/kg to at least about 10 mg/kg, or at least about 1 mg/kg to at least about 10 mg/kg, or any intervening range thereof.

In some aspects, a therapeutically effective amount of a ligand can be expressed as a molar concentration (i.e., M or mol/L). In some cases, a therapeutically effective amount of a ligand can be about 1 nM, 2 nM, 3 nM, 4 nM, 5 nM, 6 nM, 7 nM, 8 nM, 9 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 200 mM, 300 mM, 400 mM, 500 mM, 600 mM, 700 mM, 800 mM, 900 mM, 1000 mM or greater.

A therapeutically effective amount of a ligand can be administered once or more than once each day. In some cases, a therapeutically effective amount of a ligand is administered as needed (e.g., when pain relief is needed). The ligand may be administered serially (e.g., every day without a break for the duration of the treatment regimen). In some cases, the treatment regimen can be less than a week, a week, two weeks, three weeks, a month, or greater than a month. In some cases, a therapeutically effective amount of a ligand is administered for a day, at least two consecutive days, at least three consecutive days, at least four consecutive days, at least five consecutive days, at least six consecutive days, at least seven consecutive days, at least eight consecutive days, at least nine consecutive days, at least ten consecutive days, or at least greater than ten consecutive days. In a particular case, a therapeutically effective amount of a ligand is administered for three consecutive days. In some cases, a therapeutically effective amount of a ligand can be administered one time per week, two times per week, three times per week, four times per week, five times per week, six times per week, seven times per week, eight times per week, nine times per week, 10 times per week, 11 times per week, 12 times per week, 13 times per week, 14 times per week, 15 times per week, 16 times per week, 17 times per week, 18 times per week, 19 times per week, 20 times per week, 25 times per week, 30 times per week, 35 times per week, 40 times per week, or greater than 40 times per week. In some cases, a therapeutically effective amount of a ligand can be administered one time per day, two times per day, three times per day, four times per day, five times per day, six times per day, seven times per day, eight times per day, nine times per day, 10 times per day, or greater than 10 times per day. In some cases, a therapeutically effective amount of a ligand is administered at least every hour, at least every two hours, at least every three hours, at least every four hours, at least every five hours, at least every six hours, at least every seven hours, at least every eight hours, at least every nine hours, at least every 10 hours, at least every 11 hours, at least every 12 hours, at least every 13 hours, at least every 14 hours, at least every 15 hours, at least every 16 hours, at least every 17 hours, at least every 18 hours, at least every 19 hours, at least every 20 hours, at least every 21 hours, at least every 22 hours, at least every 23 hours, or at least every day. The dose of ligand may be administered to the subject continuously, or 1, 2, 3, 4, or 5 times a day; 1, 2, 3, 4, 5, 6, or 7 times a week, 1, 2, 3, or 4 times a month, once every 2, 3, 4, 5, or 6 months, or once a year, or at even longer intervals. The duration of treatment can last a day, 1, 2, or 3 weeks, 1, 2, 3, 4, 5, 7, 8, 9, 10, or 11 months, 1, 2, 3, 4, 5, or more years, or longer.

A subject treated by methods and compositions disclosed herein can be a human, or can be a non-human animal. The term “treat” and its grammatical equivalents used herein generally refer to the use of a composition or method to reduce, eliminate, or prevent symptoms of a disease and includes achieving a therapeutic benefit and/or a prophylactic benefit. By “therapeutic benefit” is meant eradication or amelioration of the underlying disorder or condition being treated. A prophylactic benefit of treatment includes reducing the risk of a condition, retarding the progress of a condition, or decreasing the likelihood of occurrence of a condition.

Non-limiting examples of non-human animals include a non-human primate, a livestock animal, a domestic pet, and a laboratory animal. For example, a non-human animal can be an ape (e.g., a chimpanzee, a baboon, a gorilla, or an orangutan), an old world monkey (e.g., a rhesus monkey), a new world monkey, a dog, a cat, a bison, a camel, a cow, a deer, a pig, a donkey, a horse, a mule, a lama, a sheep, a goat, a buffalo, a reindeer, a yak, a mouse, a rat, a rabbit, or any other non-human animal. The compositions and methods as described herein are amenable to the treatment of a veterinary animal. A veterinary animal can include, without limitation, a dog, a cat, a horse, a cow, a sheep, a mouse, a rat, a guinea pig, a hamster, a rabbit, a snake, a turtle, and a lizard.

### K. Kits

Compositions and reagents useful for the present invention may be packaged in kits to facilitate application of particular embodiments of the present invention. In some embodiments, a kit is provided comprising a polynucleotide, vector, or composition contemplated herein. In one embodiment, the kit comprises a recombinant virus contemplated herein. Embodiments of the kit contemplated herein may also comprised instructions. The instructions could be in any desired form, including but not limited to, printed on a kit insert, printed on one or more containers, as well as electronically stored instructions provided on an electronic storage medium, such as a computer readable storage medium.

Kits may comprise any component suitable to perform the methods of the present disclosure. In one case, a kit may comprise a biopharmaceutical composition. The biopharmaceutical composition may be provided in one or more therapeutically effective dose. In one case, the biopharmaceutical composition may include a vector encoding a GPCR or LGIC. In other cases, the kit may include an empty vector and reagents suitable to clone a GPCR or LGIC of the disclosure into the vector. Non-limiting examples of reagents suitable for cloning a GPCR or LGIC may include reagents for amplifying a GPCR or LGIC nucleic acid sequence such as template DNA or RNA, reverse transcriptases, primers, dNTPs, DNA polymerases, and buffers; reagents for cloning the GPCR or LGIC such as restriction endonucleases (i.e., restriction enzymes), DNA ligases, and buffers.

In some cases, the kit may include a one or more therapeutically effective dose of a ligand as described herein. In some cases, the kit includes one or more therapeutically effective dose of clozapine-N-oxide. In other cases, the kit includes one or more therapeutically effective dose of salvinorin B.

The kit may comprise a therapeutically effective dose of any composition in a tablet formulation for oral administration. In other cases, the kit may comprise a therapeutically effective dose of any composition in a liquid formulation for intrathecal, intraganglionic, intraneural, intracranial, intrapsinal or subcutaneous administration. A composition may be provided in any formulation as described herein (i.e., a tablet, a gel, a cream, and the like). In some cases, the kit may comprise any composition in a dried (i.e., lyophilized) or powdered form. The dried or powdered drug may be reconstituted with a liquid solution (i.e., a saline solution) to form a liquid formulation. The vector and ligand compositions may be provided separately (e.g., in separate kits). Kits may further comprise one or more excipients as described herein (i.e., a preservative, a carrier, etc).

The kit may further comprise any device suitable for administration of the composition. For example, a kit comprising an injectable formulation of the pharmaceutical compositions may comprise a needle suitable for subcutaneous administration and an alcohol wipe for sterilization of the injection site.

In some cases, kits may comprise reagents and materials for drug discovery. Kits of this nature may contain cells suitable for screening compounds. In some cases, the cells may be primary neurons, astrocytes, or glial cells. In some cases, the cells are neurons generated from neural stem cells or neural progenitor cells. The neurons may be generated from induced pluripotent stem cells (iPS). iPS cells may be engineered cells (e.g., fibroblasts, skin cells, and the like) that have reverted to a pluripotent state. In some cases, the iPS cells may be derived from a subject or patient. In some cases, the patient may suffer from a disease. In some cases, iPS cells may be provided in the kit with reagents and instructions for generating neurons. Cells as described herein may be provided in a vial (e.g., in a frozen state) or may be provided on a dish ready for culturing. Kits may comprise cell culture media suitable for growing the cells of the disclosure. In other cases, the kit may comprise instruments and reagents for collecting cells from a subject and for generating iPS cells. For example, the kit may comprise a tool for collecting skin cells (or a skin biopsy) from a subject and a set of reagents for generating iPS cells from the collected skin cells (e.g., a transfection reagent, a set of plasmids for expression of iPS marker genes (e.g., Oct4, SOX2, NANOG, etc.)). Kits for screening compounds may contain a vector or nucleic acid molecule encoding a GPCR or LGIC. In some cases, kits may comprise one or more candidate compounds for screening. In some cases, the candidate compounds are ligands for a GPCR or LGIC. In particular examples, kits may comprise reagents and tools for screening compounds that activate a target GPCR or LGIC in a neuron. The kit may further comprise tools for measuring neuronal activity in cell culture conditions (e.g., patch clamp system, voltage-sensitive dyes, and the like).

In some cases, kits may be provided with instructions. The instructions may be provided in the kit or they may be accessed electronically (e.g., on the World Wide Web). The instructions may provide information on how to use the compositions of the present disclosure. The instructions may further provide information on how to use the devices of the present disclosure. The instructions may provide information on how to perform the methods of the disclosure. In some cases, the instructions may provide dosing information. In some cases, the instructions may provide drug information such as the mechanism of action, the formulation of the drug, adverse risks, contraindications, and the like. In some cases, the kit is purchased by a physician or health care provider for administration at a clinic or hospital. In some cases, the kit is purchased by a laboratory and used for screening candidate compounds.

The present invention now will be described more fully by the following examples. This invention may, however, be embodied in many different forms and should not be construed as limited to the embodiments set forth herein; rather, these embodiments are provided so that this disclosure will be thorough and complete, and will fully convey the scope of the invention to those skilled in the art.

TABLE 4
Exemplary Gene Therapy Vector
Nucleotide Sequence

| SEQ | | |
|---|---|---|
| ID | | |
| NO | NAME | SEQUENCE |
| 1 | hSYN1- | GACGCGCCCTGTAGCGGCGCATTAAGCGCGGCGGGTG |
| | GlyRα1 | TGGTGGTTACGCGCAGCGTGACCGCTACACTTGCCAG |
| | F207A/ | CGCCCTAGCGCCCGCTCCTTTCGCTTTCTTCCCTTCC |
| | A288G | TTTCTCGCCACGTTCGCCGGCTTTCCCCGTCAAGCTC |
| | | TAAATCGGGGGCTCCCTTTAGGGTTCCGATTTAGTGC |
| | | TTTACGGCACCTCGACCCCAAAAAACTTGATTAGGGT |
| | | GATGGTTCACGTAGTGGGCCATCGCCCTGATAGACGG |
| | | TTTTTCGCCCTTTGACGTTGGAGTCCACGTTCTTTAA |
| | | TAGTGGACTCTTGTTCCAAACTGGAACAACACTCAAC |
| | | CCTATCTCGGTCTATTCTTTTGATTTATAAGGGATTT |
| | | TGCCGATTTCGGCCTATTGGTTAAAAAATGAGCTGAT |
| | | TTAACAAAAATTTAACGCGAATTTTAACAAAATATTA |
| | | ACGTTTACAATTTCAGGTGGCACTTTTCGGGGAAATG |
| | | TGCGCGGAACCCCTATTTGTTTATTTTTCTAAATACA |
| | | TTCAAATATGTATCCGCTCATGAGACAATAACCCTGA |
| | | TAAATGCTTCAATAATATTGAAAAAGGAAGAGTATGA |
| | | GTATTCAACATTTCCGTGTCGCCCTTATTCCCTTTTT |
| | | TGCGGCATTTTGCCTTCCTGTTTTTGCTCACCCAGAA |
| | | ACGCTGGTGAAAGTAAAAGATGCTGAAGATCAGTTGG |
| | | GTGCACGAGTGGGTTACATCGAACTGGATCTCAACAG |
| | | CGGTAAGATCCTTGAGAGTTTTCGCCCCGAAGAACGT |
| | | TTTCCAATGATGAGCACTTTTAAAGTTCTGCTATGTG |
| | | GCGCGGTATTATCCCGTATTGACGCCGGGCAAGAGCA |
| | | ACTCGGTCGCCGCATACACTATTCTCAGAATGACTTG |
| | | GTTGAGTACTCACCAGTCACAGAAAAGCATCTTACGG |
| | | ATGGCATGACAGTAAGAGAATTATGCAGTGCTGCCAT |
| | | AACCATGAGTGATAACACTGCGGCCAACTTACTTCTG |
| | | ACAACGATCGGAGGACCGAAGGAGCTAACCGCTTTTT |
| | | TGCACAACATGGGGGATCATGTAACTCGCCTTGATCG |
| | | TTGGGAACCGGAGCTGAATGAAGCCATACCAAACGAC |
| | | GAGCGTGACACCACGATGCCTGTAGCAATGGCAACAA |
| | | CGTTGCGCAAACTATTAACTGGCGAACTACTTACTCT |
| | | AGCTTCCCGGCAACAATTAATAGACTGGATGGAGGCG |
| | | GATAAAGTTGCAGGACCACTTCTGCGCTCGGCCCTTC |
| | | CGGCTGGCTGGTTTATTGCTGATAAATCTGGAGCCGG |
| | | TGAGCGTGGGTCTCGCGGTATCATTGCAGCACTGGGG |
| | | CCAGATGGTAAGCCCTCCCGTATCGTAGTTATCTACA |
| | | CGACGGGGAGTCAGGCAACTATGGATGAACGAAATAG |
| | | ACAGATCGCTGAGATAGGTGCCTCACTGATTAAGCAT |
| | | TGGTAACTGTCAGACCAAGTTTACTCATATATACTTT |
| | | AGATTGATTTAAAACTTCATTTTTAATTTAAAAGGAT |
| | | CTAGGTGAAGATCCTTTTTGATAATCTCATGACCAAA |
| | | ATCCCTTAACGTGAGTTTTCGTTCCACTGAGCGTCAG |
| | | ACCCCGTAGAAAAGATCAAAGGATCTTCTTGAGATCC |
| | | TTTTTTTCTGCGCGTAATCTGCTGCTTGCAAACAAAA |
| | | AAACCACCGCTACCAGCGGTGGTTTGTTTGCCGGATC |
| | | AAGAGCTACCAACTCTTTTTCCGAAGGTAACTGGCTT |
| | | CAGCAGAGCGCAGATACCAAATACTGTCCTTCTAGTG |
| | | TAGCCGTAGTTAGGCCACCACTTCAAGAACTCTGTAG |
| | | CACCGCCTACATACCTCGCTCTGCTAATCCTGTTACC |
| | | AGTGGCTGCTGCCAGTGGCGATAAGTCGTGTCTTACC |
| | | GGGTTGGACTCAAGACGATAGTTACCGGATAAGGCGC |
| | | AGCGGTCGGGCTGAACGGGGGGTTCGTGCACACAGCC |
| | | CAGCTTGGAGCGAACGACCTACACCGAACTGAGATAC |
| | | CTACAGCGTGAGCATTGAGAAAGCGCCACGCTTCCCG |
| | | AAGGGAGAAAGGCGGACAGGTATCCGGTAAGCGGCAG |
| | | GGTCGGAACAGGAGAGCGCACGAGGGAGCTTCCAGGG |
| | | GGAAACGCCTGGTATCTTTATAGTCCTGTCGGGTTTC |
| | | GCCACCTCTGACTTGAGCGTCGATTTTTGTGATGCTC |
| | | GTCAGGGGGGCGGAGCCTATGGAAAAACGCCAGCAAC |
| | | GCGGCCTTTTTACGGTTCCTGGCCTTTTGCTGGCCTT |
| | | TTGCTCACATGTTCTTTCCTGCGTTATCCCCTGATTC |
| | | TGTGGATAACCGTATTACCGCCTTTGAGTGAGCTGAT |
| | | ACCGCTCGCCGCAGCCGAACGACCGAGCGCAGCGAGT |
| | | CAGTGAGCGAGGAAGCGGAAGAGCGCCTGATGCGGTA |
| | | TTTTCTCCTTACGCATCTGTGCGGTATTTCACACCGC |
| | | AGACCAGCCGCGTAACCTGGCAAAATCGGTTACGGTT |
| | | GAGTAATAAATGGATGCCCTGCGTAAGCGGGTGTGGG |
| | | CGGACAATAAAGTCTTAAACTGAACAAAATAGATCTA |
| | | AACTATGACAATAAAGTCTTAAACTAGACAGAATAGT |
| | | TGTAAACTGAAATCAGTCCAGTTATGCTGTGAAAAAG |
| | | CATACTGGACTTTTGTTATGGCTAAAGCAAACTCTTC |
| | | ATTTTCTGAAGTGCAAATTGCCCGTCGTATTAAAGAG |
| | | GGGCGTGGCCAAGGGCATGGTAAAGACTATATTCGCG |
| | | GCGTTGTGACAATTTACCGAACAACTCCGCGGCCGGG |
| | | AAGCCGATCTCGGCTTGAACGAATTGTTAGGTGGCGG |
| | | TACTTGGGTCGATATCAAAGTGCATCACTTCTTCCCG |
| | | TATGCCCAACTTTGTATAGAGAGCCACTGCGGGATCG |
| | | TCACCGTAATCTGCTTGCACGTAGATCACATAAGCAC |
| | | CAAGCGCGTTGGCCTCATGCTTGAGGAGATTGATGAG |
| | | CGCGGTGGCAATGCCCTGCCTCCGGTGCTCGCCGGAG |
| | | ACTGCGAGATCATAGATATAGATCTCACTACGCGGCT |
| | | GCTCAAACCTGGGCAGAACGTAAGCCGCGAGAGCGCC |
| | | AACAACCGCTTCTTGGTCGAAGGCAGCAAGCGCGATG |
| | | AATGTCTTACTACGGAGCAAGTTCCCGAGGTAATCGG |
| | | AGTCCGGCTGATGTTGGGAGTAGGTGGCTACGTCTCC |
| | | GAACTCACGACCGAAAAGATCAAGAGCAGCCCGCATG |
| | | GATTTGACTTGGTCAGGGCCGAGCCTACATGTGCGAA |
| | | TGATGCCCATACTTGAGCCACCTAACTTTGTTTTAGG |
| | | GCGACTGCCCTGCTGCGTAACATCGTTGCTGCTGCGT |
| | | AACATCGTTGCTGCTCCATAACATCAAACATCGACCC |
| | | ACGGCGTAACGCGCTTGCTGCTTGGATGCCCGAGGCA |
| | | TAGACTGTACAAAAAAACAGTCATAACAAGCCATGAA |
| | | AACCGCCACTGCGCCGTTACCACCGCTGCGTTCGGTC |
| | | AAGGTTCTGGACCAGTTGCGTGAGCGCATACGCTACT |
| | | TGCATTACAGTTTACGAACCGAACAGGCTTATGTCAA |
| | | CTGGGTTCGTGCCTTCATCCGTTTCCACGGTGTGCGT |
| | | CACCCGGCAACCTTGGGCAGCAGCGAAGTCGAGGCAT |
| | | TTCTGTCCTGGCTGGCGAACGAGCGCAAGGTTTCGGT |
| | | CTCCACGCATCGTCAGGCATTGGCGGCCTTGCTGTTC |
| | | TTCTACGGCAAGGTGCTGTGCACGGATCTGCCCTGGC |
| | | TTCAGGAGATCGGAAGACCTCGGCCGTCGCGGCGCTT |
| | | GCCGGTGGTGCTGACCCCGGATGAAGTGGTTCGCATC |
| | | CTCGGTTTTCTGGAAGGCGAGCATCGTTTGTTCGCCC |
| | | AGGACTCTAGCTATAGTTCTAGTGGTTGGCTACAGCT |
| | | TGCTGCGCGCTCGCTCGCTCACTGAGGCCGCCCGGGC |
| | | AAAGCCCGGGCGTCGGGCGACCTTTGGTCGCCCGGCC |
| | | TCAGTGAGCGAGCGAGCGCGCAGAGAGGGAGTGGGGT |
| | | TGATCTCTCCCCAGCATGCCTGCTATTGTCTTCCCAA |
| | | TCCTCCCCCTTGCTGTCCTGCCCCACCCCACCCCCCA |
| | | GAATAGAATGACACCTACTCAGACAATGCGATGCAAT |
| | | TTCCTCATTTTATTAGGAAAGGACAGTGGGAGTGGCA |
| | | CCTTCCAGGGTCAAGGAAGGCACGGGGGAGGGGCAAA |
| | | CAACAGATGGCTGGCAACTAGAAGGCACAGTCGAGGC |
| | | TGATCAGCGAGCTCTAGTCGACGGTATCGATTTAAAC |
| | | CTTATCGTCGTCATCCTTGTAATCGAGCGGCCGCGTA |
| | | CGCGTCTGGTTGTGGACGTCCTCTCTACGGACAATCT |
| | | TGTAGATGATCCAGTAGAACATGTTGAAAATGAGGAA |
| | | GGCCATGGGGAAGCCAATGCGGGATATTTTGTCGATC |
| | | TTCTTGGCCCTCTGGATGAAGAGTTTTCGCATCTCCT |
| | | CTGGGGACTTAGATGGTGCAGGAGGGGGGTTGGTGGT |
| | | GTTACTGTTGTTGGCGCCCTTGACTGAGATGCCATCC |
| | | TTGGCCTGTAGACAGGCTGGGCCCATCCCATAGGCAG |
| | | AGAAGTTAAAGCGGCCTTCTCCAGCTTCATCCTCCTG |
| | | GAATAGATTCAACATGGGGCTCTTGTGATGTCTCCGC |
| | | TTCCTCCTGAATCGGAGCAGCTCCTTATGTTGCCGAG |
| | | ACACAAAGTTAACGGCAGCATATTCTAATAGGGCTGA |
| | | GAACACAAAGAGCAGGCAAACTCCCATCCAAATGTCA |
| | | ATGGCTTTCACATAGGACACCTTGGGCAGAGATGCTC |
| | | GAGAGCCGGAGCTCTGGGTGGTCATGGTGAGCACAGT |
| | | GGTGATGCCTAGGCCCACACGAGCAGGTGCAGCATCC |
| | | ATGTTGATCCAGAAGGAGATCCATGAGAGGATGACAA |
| | | TGAGCAGGCTGGGAATATACATCTGAATCAGGTAGTA |
| | | ACCCATCTGCCGCTCCAGGTGGAACCGGGCCTCAATG |
| | | CAGGTGGCTTTACCTGTGTTGTAGTGCTTGGTGCAGT |
| | | ATCTCAAGTCCTTCTCTTCCTTCAAGATAAACTGGGG |
| | | CAGAGTTAGTCCATCTGCTACCTGCACGGCTCCCTGT |
| | | TCCTGCCACTCAAAGATGAGGTCATTCATCGTATATC |
| | | CAAAGCTTTCCAGTTGCATGATACATGTCTGGACATC |
| | | CATGGGGAAATTCTTCAAGTCCATGGGGCAGGCCAGT |
| | | GTCAGGGTGATTCTGATGCTGTAGAGGACATTCCCAT |
| | | TCCGGGAGATCCTTAGCAATTTGTTGTCTGTGGTGAT |
| | | CTCATGGAAGTGGGCCCCCTTCTCGTTGGCAAAGAAC |
| | | AGGTCAGGTTTCCAGATGGAGTCCAGCATGGATGGGT |
| | | CCAGGTCCAGAGAGTCGTCAGGGTATTCATTATAGGC |
| | | CAGGCGGGGGTCGTTCCATTGCTGCCGCAGGAAGATG |
| | | TTGACCCTATAGTCCATGGTTGTCTCAGCAATGGAAC |
| | | CAAAGCTGTTGATGAAAATGTTGCAGCTCACGTTCAC |
| | | TGGGGGACCTTTAAAATTGGGCCTGATCCTGGCATCA |
| | | TATCCGGAGGTTCTCCCCATTAGCTTATCCAGGAAAT |
| | | CCGAGGGTGACATAGGCTTGGGTGCGGAGCGAGCCAT |
| | | GGTGGCTAGCAGCTTGAATTCTCGACTGCGCTCTCAG |
| | | GCACGACACGACTCCTCCGCTGCCCACCGCAGACTGA |
| | | GGCAGCGCTGAGTCGCCGGCGCCGCAGCGCAGATGGT |
| | | CGCGCCCGTGCCCCCCTATCTCGCGCCTCGCGTGGTG |
| | | CGGTCCGGCTGGGCCGGCGGCGGCGCGGACGCGACCA |
| | | AGGTGGCCGGGAAGGGGAGTTTGCGGGGGACCGGCGA |
| | | GTGACGTCAGCGCGCCTTCAGTGCTGAGGCGGCGGTG |
| | | GCGCGCGCCGCCAGGCGGGGGCGAAGGCACTGTCCGC |
| | | GGTGCTGAAGCTGGCAGTGCGCACGCGCCTCGCCGCA |
| | | TCCTGTTTCCCCTCCCCCTCTCTGATAGGGGATGCGC |
| | | AATTTGGGGAATGGGGGTTGGGTGCTTGTCCAGTGGG |
| | | TCGGGGTCGGTCGTCAGGTAGGCACCCCCACCCCGCC |
| | | TCATCCTGGTCCTAAAACCCACTTGCACTCATACGCA |
| | | GGGCCCTCTGCAGTCTAGTGGTACCGAATTCAGATCT |
| | | GCAGCTTCCTAGGAACCCCTAGTGATGGAGTTGGCCA |
| | | CTCCCTCTCTGCGCGCTCGCTCGCTCACTGAGGCCGG |
| | | GCGACCAAAGGTCGCCCGACGCCCGGGCTTTGCCCGG |
| | | GCGGCCTCAGTGAGCGAGCGAGCGCGCCAGGATCCGA |
| | | GCTTGTCGAGAAGTACTAGAGGATCATAATCAGCCAT |
| | | ACCACATTTGTAGAGGTTTTACTTGCTTTAAAAAACC |
| | | TCCCACACCTCCCCCTGAACCTGAAACATAAAATGAA |
| | | TGCAATTGTTGTTGTTAACTTGTTTATTGCAGCTTAT |
| | | AATGGTTACAAATAAAGCAATAGCATCACAAATTTCA |
| | | CAAATAAAGCATTTTTTTCACTGCATTCTAGTTGTGG |
| | | TTTGTCCAAACTCATCAATGTATCTTATCATGTCTGG |
| | | ATCTGATCACTGCTTGAGCCTAGGAGATCCGAACCAG |
| | | ATAAGTGAAATCTAGTTCCAAACTATTTTGTCATTTT |
| | | TAATTTTCGTATTAGCTTACGACGCTACACCCAGTTC |
| | | CCATCTATTTTGTCACTCTTCCCTAAATAATCCTTAA |
| | | AAACTCCATTTCCACCCCTCCCAGTTCCCAACTATTT |
| | | TGTCCGCCCACAGCGGGGCATTTTTCTTCCTGTTATG |
| | | TTTTTAATCAAACATCCTGCCAACTCCATGTGACAAA |
| | | CCGTCATCTTCGGCTACTTTTTCTCTGTCACAGAATG |
| | | AAAATTTTTCTGTCATCTCTTCGTTATTAATGTTTGT |
| | | AATTGACTGAATATCAACGCTTATTTGCAGCCTGAAT |
| | | GGCGAATG |

### EXAMPLES

### Example 1. Construction of hSYN1—GlyR αlpha1 F207A/A288G AAV Vectors

### Cloning of V272-pFB-inCap6(Y705+Y731F+T492V)-inRep-Kan

A 390 bp cap6 fragment containing an introduced SbfI site and the mutation (T492V) is PCR amplified with primers 2793F and 2794R. A 440 bp cap6 fragment containing an introduced BsiWI site and the mutation (T492V) is PCR amplified with primers 2795F and 2796R. The amplification products are isolated by gel electrophoresis and purified.

The purified 390 bp and 440 bp PCR products are subjected to overlap PCR to generate a 810 bp cap6 fragment with primers 2793F and 2796R. The amplification product is isolated by gel electrophoresis and purified.

The purified 810 bp cap6 fragment is digested with SbfI and BsiWI and ligated into a V220 vector digested with SbfI and BsiWI to generate V272-pFB-inCap6(Y705+731F+T492V)-inRep-Kan.

### Primer Sequences:

| Primer | Primer Sequence 5′ to 3′ | SEQ ID NO: |
|---|---|---|
| 2793F | ATAGGACCCTGCAGGTATAC | 16 |
| 2794R | CGTTTCTAAAGTAAAAACAGACAACAACAA | 17 |
| 2795F | CTGTTTTTACTTTAGAAACGCGCTGCTGCC | 18 |
| 2796R | GTACCAGTTGCCGTACGTCC | 19 |

### Cloning of SWB01-pFB-hSyn-Gly(F207A+A288G)

The following fragments are PCR amplified: (a) the hSyn promoter (510 bp) is PCR amplified with primers 2799F and 2800R using hSYN plasmid as template; (b) a Glycine receptor alpha 1 fragment (652 bp) is PCR amplified with primers 2801F and 2802R using wild type Glycine receptor alpha 1 plasmid as template; (c) a Glycine receptor alpha 1 fragment (266 bp) is PCR amplified with primers 2803F and 2804R and wild type Glycine receptor alpha 1 plasmid as template; (d) a Glycine receptor alpha 1 fragment (461 bp) is PCR amplified with primers 2805F and 2806R and wild type Glycine receptor alpha 1 plasmid as template; and (e) a bGHpA fragment (282 bp) is PCR amplified with primers 2807F, 2808F and 2809R using V261 as a template. The amplification products are isolated by gel electrophoresis and purified

The purified PCR fragments are used in an overlap PCR to generate the following larger fragments: (1) fragments (a), (b), and (c) are joined together (1338 bp) using overlap PCR with primers 2799F and 2804R; and (2) fragments (c), (d), and (e) are joined together (971 bp) using overlap PCR with primers 2803F and 2809R. The amplification products are isolated by gel electrophoresis and purified.

Fragment 1 is digested with KpnI and AvrII and fragment 2 is digested with AvrII and SphI. These fragments are ligated into pFB-sc-EGFP digested with KpnI and SphI to generate SWB01-pFB-hSyn-Gly(F207A+A288G).

### Primer Sequences:

| | | SEQ |
|---|---|---|
| Primer | Primer Sequence 5′ to 3′ | ID NO: |
| 2799F | GATCTGGTACCACTAGACTGCAGAGGGCCC | 20 |
| 2800R | GTGGCTAGCAGCTTGAATTCTCGACTGCGCTCTC | 21 |
| | AGGCAC | |
| 2801F | GAATTCAAGCTGCTAGCCACCATGGCTCGCTCCG | 22 |
| | CACCCA | |
| 2802R | TGCAGGTGGCTTTACCTGTGTTGTAGTGCT | 23 |
| 2803F | CACAGGTAAAGCCACCTGCATTGAGGCCCG | 24 |
| 2804R | GCAGGCAAACTCCCATCCAAATGTCAATGG | 25 |
| 2805F | GGATGGGAGTTTGCCTGCTCTTTGTGTTCT | 26 |
| 2806R | ATCCTTGTAATCGAGCGGCCGCGTACGCGTCTGG | 27 |
| | TTGTGGACGTCCTCTC | |
| 2807F | GGCCGCTCGATTACAAGGATGACGACGATAAGGT | 28 |
| | TTAAATCGATACCGTC | |
| 2808F | AAGGTTTAAATCGATACCGTCGACTAGAGCTCGC | 29 |
| | TGATCAGCCTCGACTG | |
| 2809R | CCCAGCATGCCTGCTATTGTCTTCCCAATC | 30 |

Recombinant baculovirus is generated to produce AAV6-Gly(hSYN-GlyR(F207A/A288G)-FLAG and AAV6(Y705F+Y731F+T492V)-Gly(hSYN-GlyR(F207A/A288G)-FLAG at 1×10¹³ vector genomes (vg)/mL.

### Example 2. Treatment of Rodent Models of Chronic Pain

### Chronic Pain Induction and Treatment

Day 0: Chronic pain is induced in rodent trigeminal ganglia or dorsal root ganglia using an established peripheral nerve injury method such as the chronic constriction injury (CCI, CO/CFA) or spared nerve injury (SNI) models. See Bennett & Xie. Pain. 1988, Decosterd & Woolf Pain. 2000, and Imamura, Kawamoto, & Nakanishi. Exp. Brain Res. 1997. In some instances, nerve injury may occur after viral vector injection.

Day 7: Intraganglionic or intrathecal injection of 10⁸-10¹⁰ vector genomes of AAV6(Y705F+Y731F+T492V)-HSYN-GLYR(F207A/A288G)-FLAG or AAV6-GLY(HSYN-GLYR(F207A/A288G)-FLAG in a volume of approximately 1.0-10 μL is performed in one or multiple dorsal root ganglia or trigeminal ganglia using published methods. See Vit, Ohara, Sundberg et al. Mol Pain. 2009 and Towne, Fischer, Kostic, et al. J Neurosci Methods. 2011, and Pertin, et al. Mol Pain. 2009.

Weeks 2-12 post CCI or SNI: Ivermectin is administered via oral gavage (P0), intraperitoneal injection (IP), subcutaneous injection (SC), or in drinking water for a final dose of 0.1-10.0 mg/kg per day. Pain and anxiety behavioral assays are performed to quantify level of analgesia using established nociception assay including operant assays, mechanical allodynia/hyperalgesia via Von Frey filaments, thermal allodynia/hyperalgesia via Hargreaves method, or anxiety behaviors via elevated plus maze. See review Odd-Geir Berge. Br J Pharmacol. 2011.

Pain related behaviors or anxiety will be measurably reduced only in the presence of ivermectin with a quantifiable improvement of 10%-500% over controls. Analgesia or reduction in pain related behaviors will not be detectable in saline controls not receiving ivermectin.

### Gene Expression Assay

Weeks 1-52 post viral vector injection: Following injection of AAV6(Y705F+Y731F+T492V)-HSYN-GLYR(F207A/A288G)-FLAG or AAV6-GLY(HSYN-GLYR(F207A/A288G)-FLAG, ganglia are harvested, processed, sectioned, and microscopically analyzed for GlyR-FLAG transgene expression with an anti-FLAG antibody, and counterstained with antibodies to NF200, Peripherin, D34, Substance P, TRPV1, CGRP, TrkA, Advillin, ATF3, Iba1, NPY, PKCy, or GFAP.

Gene expression will be detectable and localized primarily to neurons and largely absent from surrounding non-neuronal tissues and cells. Gene expression will not be present in contralateral control tissues.

### Example 3. Treatment of a Patient Suffering from Chronic Pain

A patient suffering from chronic pain is treated using the compositions and methods disclosed herein. The patient is treated on Day One with 10¹⁵ vector genomes of AAV-hSYN1-hM4Di in a volume of 12.0 mL into the subarachnoid space of the spinal cord (i.e., intrathecal). In this example, the AAV vector encodes the human muscarinic DREADD, hM4Di, under the control of the human Synapsin-1 (SYN1) promoter for selective neuronal expression. Two weeks post-injection, the patient returns to the clinic for a prescription for clozapine-N-oxide (CNO). The patient self-administers 100 μM CNO orally as needed (i.e., during a pain episode).

### Example 4. Treatment of a Patient Suffering from Chronic Pain

In a non-limiting example, a patient suffering from chronic radicular pain is treated using the compositions and methods disclosed herein. The patient is treated on Day One with 10¹³ vector genomes of AAV-hSYN1-GlyR-M in a volume of 1.0 mL delivered directly into one or more dorsal root ganglia (i.e., intraganglionic convection-enhanced delivery into lumbar, cervical, or thoracic DRGs). In this example, the AAV vector encodes the human glycine receptor harboring the F207A/A288G mutations, GlyR-M, under the control of the human Synapsin-1 (SYN1) promoter for selective neuronal expression. Two weeks post-injection, the patient returns to the clinic for a prescription for ivermectin (IVM). The patient self-administers 0.1 mg/kg IVM orally as needed (i.e., during a pain episode).

### Example 5. Treatment of a Patient Suffering from Chronic Pain

In a non-limiting example, a patient suffering from chronic craniofacial pain (e.g. trigeminal neuralgia or temporomandibular joint dysfunction) is treated using the compositions and methods disclosed herein. The patient is treated on Day One with 10¹³ vector genomes of AAV-hSYN1-GlyR-M in a volume of 1.0 mL delivered directly into the trigeminal ganglion (i.e., intraganglionic convection enhanced delivery). In this example, the AAV vector encodes the human glycine receptor harboring the F207A/A288G mutations, GlyR-M, under the control of the human Synapsin-1 (SYN1) promoter for selective neuronal expression. Two weeks post-injection, the patient returns to the clinic for a prescription for ivermectin (IVM). The patient self-administers 0.1 mg/kg IVM orally as needed (i.e., during a pain episode).

### Example 6. Treatment of a Patient Suffering from Chronic Pancreatic Pain

In a non-limiting example, a patient suffering from chronic pancreatic pain (e.g. pancreatitis or pancreatic cancer) is treated using the compositions and methods disclosed herein. The patient is treated on Day One with 10¹³ vector genomes of AAV-hSYN1-GlyR-M in a volume of 1.0 mL delivered directly into the celiac nerve plexus (i.e., intraneural). In this example, the AAV vector encodes the human glycine receptor harboring the F207A/A288G mutations, GlyR-M, under the control of the human Synapsin-1 (SYN1) promoter for selective neuronal expression. Two weeks post-injection, the patient returns to the clinic for a prescription for ivermectin (IVM). The patient self-administers 0.1 mg/kg IVM orally as needed (i.e., during a pain episode).

### Example 7. Treatment of a Patient Suffering from Obesity

In a non-limiting example, a patient suffering from obesity is treated using the compositions and methods disclosed herein. The patient is treated on Day One with 10¹³ vector genomes of AAV-Ghrelin-GlyR-M in a volume of 1.0 mL delivered directly into the gastric branch of the vagus nerve (i.e., intraneural). In this example, the AAV vector encodes the human glycine receptor harboring the F207A/A288G mutations, GlyR-M, under the control of the human Ghrelin promoter for selective neuronal expression. Two weeks post-injection, the patient returns to the clinic for a prescription for ivermectin (IVM). The patient self-administers 0.1 mg/kg IVM orally daily for excess weight loss (i.e. for apetitite suppression).

### Example 8. Treatment of a Patient Suffering from Obesity

In a non-limiting example, a patient suffering from obesity is treated using the compositions and methods disclosed herein. The patient is treated on Day One with 10¹³ vector genomes of AAV-TRPV1-GlyR-M in a volume of 1.0 mL delivered directly into the dorsal root ganglia innervating the pancreas (i.e., intragangionic). In this example, the AAV vector encodes the human glycine receptor harboring the F207A/A288G mutations, GlyR-M, under the control of the human TRPV1 promoter for selective neuronal expression in nociceptors. Two weeks post-injection, the patient returns to the clinic for a prescription for ivermectin (IVM). The patient self-administers 0.1 mg/kg IVM orally daily for excess weight loss.

### Example 9. Treatment of a Patient Suffering from Obesity

In a non-limiting example, a patient suffering from obesity is treated using the compositions and methods disclosed herein. The patient is treated on Day One with 10¹³ vector genomes of AAV-SIM1-hM3Dq in a volume of 1.0 mL delivered directly into the paraventricular nucleus (PVH) in the hypothalamus (i.e., intracranial, convection enhanced delivery). In this example, the AAV vector encodes the human muscarinic DREADD, hM3Dq, under the control of the human Single-Minded Family BHLH Transcription Factor 1 (SIM1) promoter for selective neuronal expression in pro-opiomelanocortin (POMC) neurons and ultimately stimulation of the anorexigenic pathway. Two weeks post-injection, the patient returns to the clinic for a prescription for clozapine. The patient self-administers 0.15 mg/kg clozapine orally daily for excess weight loss (i.e. for apetitite suppression).

### Example 10. Treatment of a Patient Suffering from Obesity

In a non-limiting example, a patient suffering from obesity is treated using the compositions and methods disclosed herein. The patient is treated on Day One with 10¹³ vector genomes of AAV-OXT-hM3Dq in a volume of 1.0 mL delivered directly into the paraventricular nucleus (PVH) in the hypothalamus (i.e., intracranial). In this example, the AAV vector encodes the human muscarinic DREADD, hM3Dq, under the control of the human Oxytocin (OXT) promoter for selective neuronal expression and ultimately stimulation of the anorexigenic pathway. Two weeks post-injection, the patient returns to the clinic for a prescription for perlapine. The patient self-administers 0.5 mg/kg perlapine (Hypnodin) orally daily for excess weight loss (i.e. for appetite suppression).

### Example 11. Treatment of a Patient Suffering from Obesity

In a non-limiting example, a patient suffering from obesity is treated using the compositions and methods disclosed herein. The patient is treated on Day One with 10¹³ vector genomes of AAV-AgRP-KORD in a volume of 1.0 mL delivered directly into the arcuate nucleus of the hypothalamus (i.e., intracranial). In this example, the AAV vector encodes the human kappa opioid receptor DREADD, KORD, under the control of the human Agouti Related Peptide (AgRP) promoter for selective neuronal expression and ultimately inhibition of the orexigenic pathway. Two weeks post-injection, the patient returns to the clinic for a prescription for salvinorin-B. The patient self-administers 0.1 mg/kg salvinorin-B orally daily for excess weight loss (i.e. for apetitite suppression).

### Example 12. Treatment of a Patient Suffering from Obesity

In a non-limiting example, a patient suffering from obesity is treated using the compositions and methods disclosed herein. The patient is treated on Day One with 10¹³ vector genomes of AAV-PKC-δ-hM3Dq in a volume of 1.0 mL delivered directly into the lateral subdivision (CEI) of the amygdala central nucleus (CEA), (i.e., intracranial). In this example, the AAV vector encodes the human muscarinic DREADD, hM3Dq, under the control of the human protein kinase C-δ (PKC-δ) promoter for selective neuronal expression in CEA GABAergic neurons. Two weeks post-injection, the patient returns to the clinic for a prescription for clozapine. The patient self-administers 0.15 mg/kg clozapine orally daily for excess weight loss (i.e. for appetite suppression).

### Example 13. Treatment of a Patient Suffering from PTSD

In a non-limiting example, a patient suffering from post-traumatic stress disorder (PTSD) is treated using the compositions and methods disclosed herein. The patient is treated on Day One with 10¹³ vector genomes of AAV-hSYN1-hM4Di in a volume of 1.0 mL delivered directly into the C6 stellate ganglion, (i.e., intraganglionic). In this example, the AAV vector encodes the human muscarinic DREADD, hM4Di, under the control of the human Synapsin-1 (hSYN1) promoter for selective neuronal expression. Two weeks post-injection, the patient returns to the clinic for a prescription for clozapine. The patient self-administers 0.15 mg/kg clozapine orally daily for PTSD symptoms (i.e. for anxiety).

### Example 14. Treatment of a Patient Suffering from Depression

In a non-limiting example, a patient suffering from treatment-resistant depression (TRD) is treated using the compositions and methods disclosed herein. The patient is treated on Day One with 10¹³ vector genomes of AAV-hSYN1-GlyR-M in a volume of 1.0 mL delivered directly into the vagus nerve, (i.e., intraneural). In this example, the AAV vector encodes the human glycine receptor harboring the F207A/A288G mutations, GlyR-M, under the control of the human Synapsin-1 (hSYN1) promoter for selective neuronal expression. Two weeks post-injection, the patient returns to the clinic for a prescription for ivermectin (IVM). The patient self-administers 0.1 mg/kg IVM orally daily for depression symptoms.

### Example 15. Treatment of a Patient Suffering from GERD

In a non-limiting example, a patient suffering from gastroesophageal reflux disease (GERD) is treated using the compositions and methods disclosed herein. The patient is treated on Day One with 10¹³ vector genomes of AAV-hSYN1-hM3Dq in a volume of 1.0 mL delivered directly into the lower esophageal sphincter (LES) vagus nerve and myenteric plexus, (i.e., intraneural). In this example, the AAV vector encodes the human muscarinic DREADD, hM3Dq, under the control of the human Synapsin-1 (hSYN1) promoter for selective neuronal expression. Two weeks post-injection, the patient returns to the clinic for a prescription for clozapine. The patient self-administers 0.15 mg/kg clozapine orally daily for symptoms of GERD (i.e. acid reflux).

### Example 16. Treatment of a Patient Suffering from GERD

In a non-limiting example, a patient suffering from gastroesophageal reflux disease (GERD) is treated using the compositions and methods disclosed herein. The patient is treated on Day One with 10¹³ vector genomes of AAV-CAG-hM3Dq in a volume of 1.0 mL delivered directly into the lower esophageal sphincter (LES) smooth muscle, (i.e., intramuscular). In this example, the AAV vector encodes the human muscarinic DREADD, hM3Dq, under the control of the hybrid chicken-beta actin (CAG) promoter for expression in LES myocytes and ultimately increased smooth muscle tone. Two weeks post-injection, the patient returns to the clinic for a prescription for clozapine. The patient self-administers 0.15 mg/kg clozapine orally daily for symptoms of GERD (i.e. acid reflux).

### Example 17. Treatment of a Patient Suffering from Epilepsy

In a non-limiting example, a patient suffering from seizures associated with epilepsy is treated using the compositions and methods disclosed herein. The patient is treated on Day One with 10¹³ vector genomes of AAV-hSYN1-GlyR-M in a volume of 1.0 mL delivered directly into the vagus nerve, (i.e., intraneural). In this example, the AAV vector encodes the human glycine receptor harboring the F207A/A288G mutations, GlyR-M, under the control of the human Synapsin-1 (hSYN1) promoter for selective neuronal expression. Two weeks post-injection, the patient returns to the clinic for a prescription for ivermectin (IVM). The patient self-administers 0.1 mg/kg IVM orally daily for epileptic symptoms (i.e. seizures).

### Example 18. Treatment of a Patient Suffering from Epilepsy

In a non-limiting example, a patient suffering from seizures associated with epilepsy is treated using the compositions and methods disclosed herein. The patient is treated on Day One with 10¹³ vector genomes of AAV-CamKIIα-KORD in a volume of 1.0 mL delivered directly into a pre-determined seizure focus such as the motor cortex (i.e., intracranial). In this example, the AAV vector encodes the human kappa opioid receptor DREADD, KORD, under the control of the human Calcium/calmodulin-dependent protein kinase II α (CamKIIα) promoter for selective neuronal expression in excitatory neurons. Two weeks post-injection, the patient returns to the clinic for a prescription for salvinorin-B. The patient self-administers 0.1 mg/kg salvinorin-B orally daily for epileptic symptoms (i.e. seizures).

### Example 19. Treatment of a Patient Suffering from a Movement Disorder

In a non-limiting example, a patient suffering from a movement disorder (e.g. Parkinsonian tremor) is treated using the compositions and methods disclosed herein. The patient is treated on Day One with 10¹³ vector genomes of AAV-CamKIIα-KORD in a volume of 1.0 mL delivered directly into the subthalamic nucleus (i.e., intracranial STN). In this example, the AAV vector encodes the human kappa opioid receptor DREADD, KORD, under the control of the human Calcium/calmodulin-dependent protein kinase II α (CamKIIα) promoter for selective neuronal expression in excitatory neurons. Two weeks post-injection, the patient returns to the clinic for a prescription for salvinorin-B. The patient self-administers 0.1 mg/kg salvinorin-B orally daily for movement disorder symptoms (i.e. tremor).

### Example 20. Treatment of Pain with Viral Vectors Expressing Switch Receptors

Use of viral vectors expressing switch receptors can be used to inhibit pain, as shown in Iyer, S. M. et al. Optogenetic and chemogenetic strategies for sustained inhibition of pain. Sci. Rep. 6, 30570; doi: 10.1038/srep30570 (2016), incorporated herein by reference in its entirety.

Briefly, the sciatic nerves of female C57BL/6 mice were injected with AAV6 vectors carrying SwiChR-eYFP under the control of the human synapsin-1 promoter. SwiChR is the step-function inhibitory channelrhodopsin. Two to three weeks following injection, strong expression of SwiChR was detected throughout the primary afferent nocireceptor. Id. Recordings from dissociated cultured dorsal root ganglia (DRGs) obtained from AAV6-hSyn-SwiChR-eYFP injected mice showed that SwiChR was responsive to a blue light pulse, and induced significant decreases in input resistance during illumination. Id. Transdermally delivered blue light affected nociceptive assays in SwiChR-expressing mice. Blinded mechanical threshold assays on mice expressing SwiChR, YFP, or the chloride-conducting inhibitory channel rhodopsin iC1C2 showed that blue light produced large, statistically significant increases in mechanical withdrawal thresholds and thermal latency measures in iC1C2+ and SwiChR+mice, but not in YFP+ mice. Id. Thermal withdrawal latency was assessed using a modified Hargreaves apparatus. In cultured, SwiChR+DRG neurons, a single 1 second blue light pulse was sufficient to induce inhibition of electrically evoked action potentials not only during the light pulse, but also for many seconds following, with high spike inhibition probabilities observed as late as 60 seconds after light stimulus. Id. As expected, optogenetic inhibition could be rapidly terminated through illumination with red light, which causes the SwiChR channel to close. Id. This ‘post-light’ inhibition property of SwiChR was retained in vivo, enabling optogenetic inhibition during the ‘post-light’ period in mice. Id. Appropriately timed supplementary light pulses could be used to extend the duration of SwiChR-mediated inhibition. Even after 1 hour of temporally sparse blue light pulses, SwiChR+ mice showed stably raised pain thresholds that were statistically indistinguishable from raised thresholds observed after a single blue light pulse. Id. YFP+ mice showed no significant change in mechanical thresholds. Id. The formalin test (a commonly used pain assay, phase I of which is primarily driven by direct activation of nociceptors, phase II of which is driven in part by inflammatory and spinal facilitation mechanisms) indicated that that optogenetic inhibition of transduced unmyelinated primary afferents was sufficient to reduce nociceptor-triggered Phase I pain behavior in SwiChR+ mice, but was insufficient to mitigate the broader inflammatory response observed in phase II. Id.

Expression of the hM4D(Gi) DREADD in primary afferent nociceptors allowed inhibition of mechanical and thermal nociception. Id. Female C57BL/6 mice were intraneurally injected with AAV6-hSyn-HA-hM4D(Gi)-IRES-mCitrine, and mCitrine expression was observed in small-diameter nociceptors. Id. Intraperitoneal clozapine-N-oxide (CNO) administration to hM4D+ mice increased mechanical withdrawal thresholds. Id. Strong inhibition of Hargreaves thresholds was observed following CNO administration to hM4D+ mice, demonstrating chemogenetic inhibition of thermal sensation. Id.

### Example 21. Treatment of Pain in Spared Nerve Injury Model with Viral Vectors Expressing Switch Receptors

### AAV Vector Production

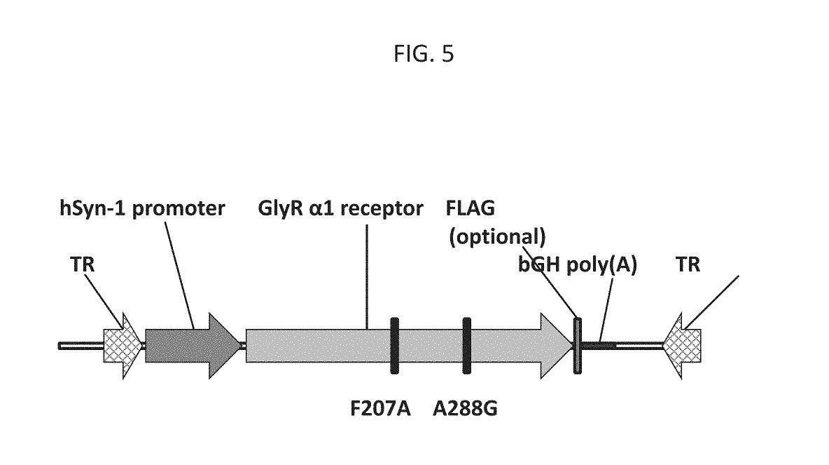

The expression cassette containing a human synapsin-1 (hSYN) promoter driving expression of the human α-1 GlyRM(F207A+A288G)-FLAG cDNA was constructed using standard molecular biology techniques (see also, FIG. 5 and SEQ ID NO:1 for description of the vector). This cassette was subcloned into a self-complementary AAV bacmid, purified, transfected into Sf9 insect cells to produce recombinant bacuvlovirus, and then amplified. Sf9 cells were double infected using the amplified recombinant baculovirus containing the hSYN-GlyRM cassette and another recombinant baculovirus containing the Rep and AAV6(Y705+731F+T492V) Cap genes to produce recombinant AAV vectors. These vectors were purified, viral titer was determined using qPCR (2.15e¹³ vg/ml), and SDS-PAGE was used to verify the purity of AAV vectors (Virovek).

AAV Spinal Cord Injection into the Dorsal Horn

A dorsal hemilaminectomy was made at the level of the lumbar enlargement to expose two segments (about 1.5-2 mm) of lumbar spinal cord, after which the dura mater was incised and reflected. The viral solution was loaded into a glass micropipette (prefilled with mineral oil). The micropipette was connected to a manual micro-injector mounted on a stereotactic apparatus. The viral solution was targeted to the dorsal horn (left side). Along the rostro-caudal axis within the exposed region, 6 injections of 240 nl each were performed, in an equidistant linear fashion. After each injection, 1 min of resting time was observed and then the muscle layer was sutured, the skin closed with staples, and the animals were allowed to recover with heated-pad before they were returned to their home cages. Animals were perfused for histological analysis after the last behavior test.

AAV Intraganglionic Injections into the Dorsal Root Ganglion (DRG)

The injection was performed with a borosilicate glass capillary (0.78/1 mm internal/external diameters) pulled to a fine point, attached by polyethylene tubing (0.4/0.8 mm internal/external diameters) to a syringe mounted in a microinjection pump. The needle was mounted on an extended arm of a stereotaxic frame swung to the outside (used to hold and manipulate the needle only). Tubing, syringe, and needle were all filled with water. One microliter air was taken up into the needle followed by 1.1 μl of the viral vector solution. The needle was loaded separately with this volume for each injection. Animals were anesthetized prior to surgery. Following an incision along the dorsal midline, the L4 and L5 DRG were exposed by removal of the lateral processes of the vertebrae. The epineurium lying over the DRG was opened, and the glass needle inserted into the ganglion, to a depth of 400 μm from the surface of the exposed ganglion. After a 3-minute delay to allow sealing of the tissue around the glass capillary tip, 1.1 μl virus solution was injected at a rate of 0.2 μl/minute. After a further delay of 2 minutes, the needle was removed. The L4 ganglion was injected first followed by the L5 ganglion. The muscles overlying the spinal cord were loosely sutured together with a 5-0 suture and the wound closed. Animals were allowed to recover at 37° C. and received postoperative analgesia.

### AAV Intrathecal Injections in Mice

Mice were first anesthetized and then placed vertically with their head fixed in a stereotaxic frame. An incision was made in the base of the neck to expose the groove in the nuchal crest. An incision was made (1-2 mm) in the cisternal membrane to a depth such that cerebrospinal fluid leaked out. A 4 cm 32 G intrathecal catheter was then slowly inserted in the direction of the lumbar spinal cord and skin was closed by suture around the catheter. The mice were then allowed to recover. Mice were then anesthetized and the vector (6 μl) was administered. The catheter was flushed with 6 μl of PBS and was then removed and mice allowed to recover.

### Behavioral Experiments and Pain Models

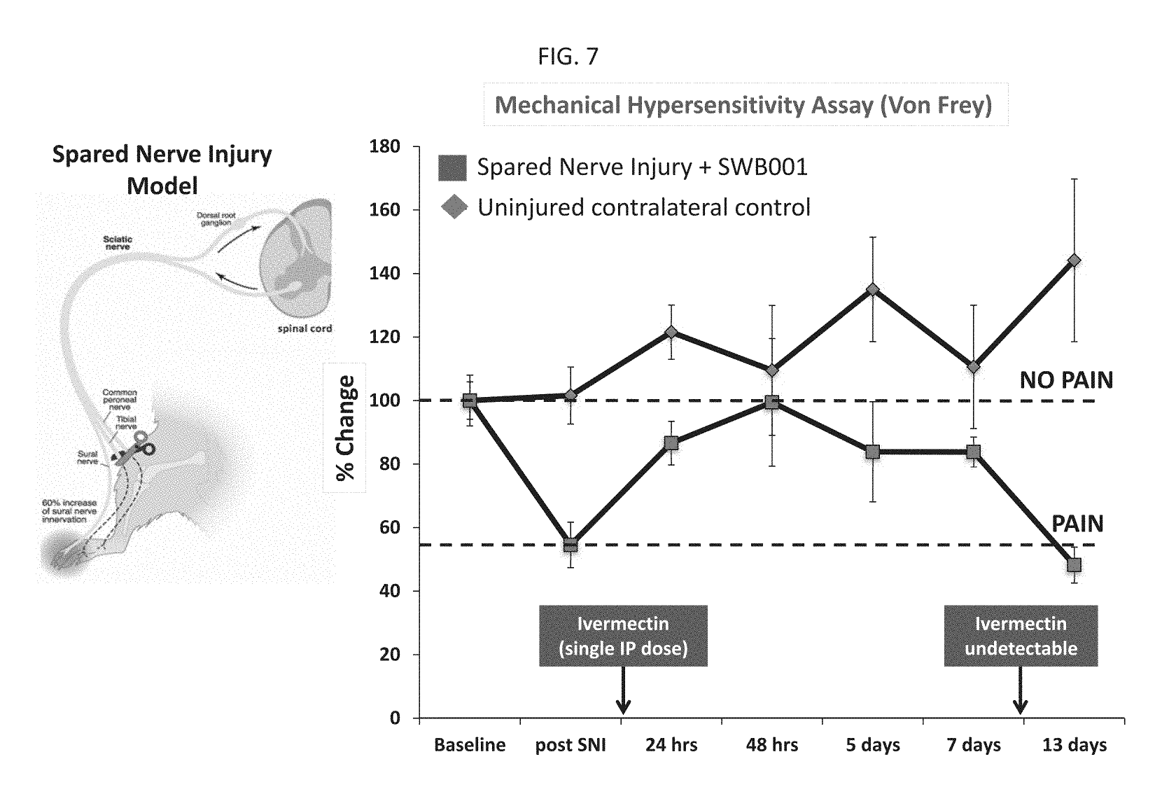

To produce mechanical hypersensitivity in a model that mimics a neuropathic pain condition, the spared nerve injury (SNI) model, a validated model of mechanical allodynia was used (Shields et al., 2003, The Journal of Pain, 4, 465-470). This model was produced by the sectioning of the common peroneal and the sural nerves and isolating the tibial branch (see FIG. 7). Mechanical withdrawal threshold was assessed by placing mice on an elevated wire-mesh grid and stimulating the plantar surface of the hindpaw with von Frey filaments. The up-down method of Chaplan & Yaksh was used to determine mechanical thresholds before the spinal cord injection of the AAV-GlyRM. Three weeks after unilateral vector injection into the dorsal horn of the spinal cord, animals were tested again to verify that their mechanical withdrawal thresholds did not change. The hindpaw ipsilateral and contralateral to the vector injection was tested. None of the animals included in the study had a change in threshold resulting from the injection of the AAV containing the GlyRM transgene. Motor coordination was tested using an accelerating rotarod (Stoelting, USA) at a maximum speed of 33 rpm. The duration that the mouse spent on the rotarod was recorded, with a cut-off at 300 sec. Each mouse went through three training trials and was tested two hours later. Subsequently, all mice received a single IP injection of ivermectin (15 mg/kg or 10 mg/kg) and mechanical thresholds were again tested using the up-down method at 1, 2, 5, 7, and 13 days post SNI. When tested with 15 mg/kg, a complete reversal of the mechanical hypersensitivity was observed. There was no change on the contralateral side, i.e. ivermectin had no measurable effect in the absence of vector. The reversal lasted for at least 5 days. On the 13th day, when the thresholds had returned to post-injury baseline, 10 mg/kg was injected IP and again a recovery to non-injury baseline thresholds was observed. These animals were followed for 48 hours and the thresholds remained at baseline. Animals were then perfused for histology.

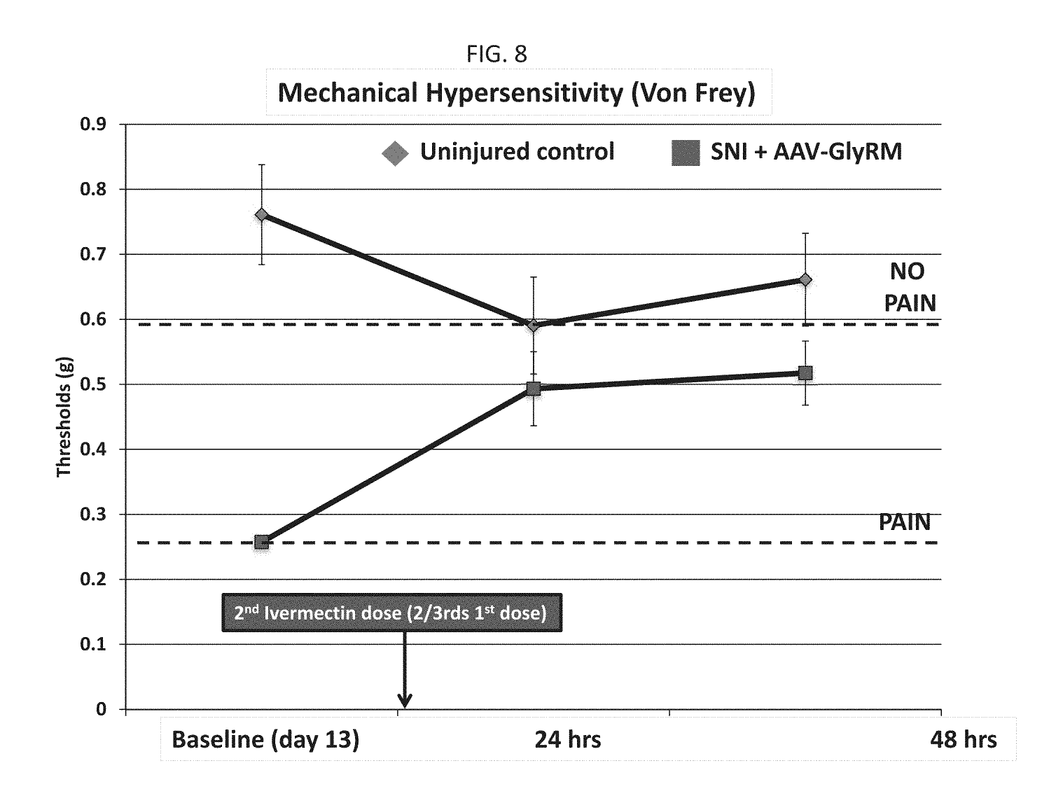

The AAV vector containing a human synapsin-1 (hSYN) promoter driving expression of the human α-1 GlyRM(F207A+A288G) (also referred to as SWB001 or AAV-GlyRM) produced 100% analgesia in the SNI model (see FIG. 7) for a duration of 7-10 days following a single IP injection of ivermectin, compared to the contralateral uninjured control side. After ivermectin washout by day 13, repeat ivermectin dosing provided 100% analgesia in the SNI model (see FIG. 8).

### Tissue Processing and Immunohistochemistry

Immunohistochemistry was performed as previously described (Braz et al., Neuron, 74(4):663-675, 2012). Briefly, mice were anesthetized with mixed saline solution of ketamine (60 mg/kg)/xylazine (8 mg/kg)/acepromazine (3 mg/kg) and perfused transcardially with 0.1 M saline phosphate buffer (PBS) followed by 4% paraformaldehyde (PFA) in PBS. Spinal cords were then extracted by laminectomy, postfixed for 2 hours in same fixative, and cryoprotected in 30% sucrose/PBS solution at 4° C. Lumbosacral dorsal root ganglia (DRGs) were also dissected, and the same postfixation and cryoprotection protocol was performed.

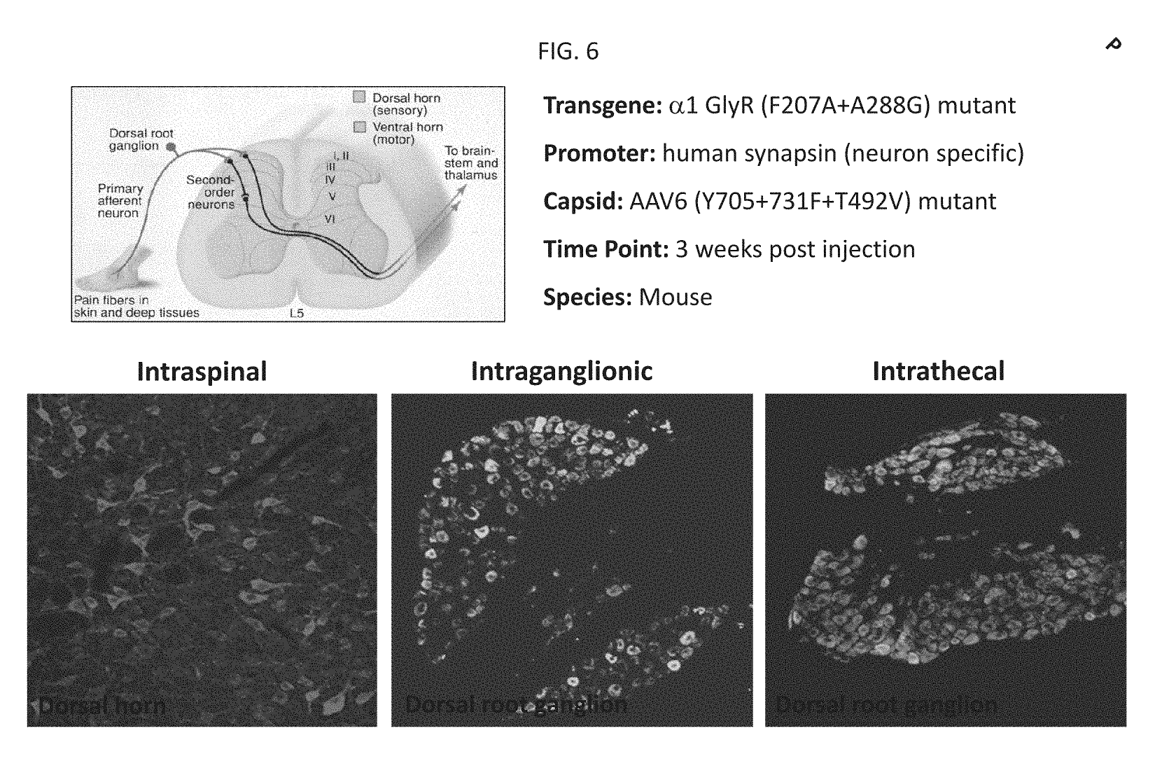

Lumbar spinal cord sections were cut using a cryostat (Leica Microsystems). Transverse sections (25 μm-thick) were cut and placed in a 48-well plate containing PBS and stored at 4° C. DRGs were embedded in TissueTek OCT compound (Bayer). Transverse sections (14 μm) were cut, mounted on Superfrost Plus slides (Thermo Fisher Scientific, Rockford, Ill.), dried for 1 hour at room temperature, and stored at 4° C. Tissue sections were washed three times in PBS/0.3% Triton X-100 (PBS-T; Sigma, St. Louis, Mo.), and incubated in 10% normal goat serum/PBS/0.3% Triton X-100 (NGST) for 1 hour, before adding the primary antibodies overnight at room temperature (RT) in a 1% NGST solution. Sections were then washed three times with PBS-T solution and incubated for 1 hour with Alexa fluorophore-conjugated secondary antibodies at RT. The sections were washed three times with PBS, air-dried, and coverslipped using Aqua Polymont (Polysciences, Inc., Warrington, USA). Primary FLAG antibody (rabbit, 1:4000; Sigma #F7425) was used to detect the GlyRM-FLAG transgene expression. Primary staining was revealed with corresponding secondary antibody conjugated to Alexa fluorophores, diluted at 1:800 in 1% NGST. Immunostained sections were imaged with Zeiss LSM700 upright confocal fluorescent microscope. Robust expression of the AAV vector containing a human synapsin-1 (hSYN) promoter driving expression of the human α-1 GlyRM(F207A+A288G) was observed three weeks post injection via multiple routes of administration (FIG. 6).

### Example 22. Effect on Thermal Pain Sensitivity of Treatment with Viral Vectors Expressing Switch Receptors

AAV injections and Thermal Withdrawal Latency Assay

On day 0, bilateral trigeminal ganglion injections of 10 microliters AAV vector containing the alpha 1 GlyR(F207A-A288G) transgene were performed in adult Sprague-Dawley rats. Two months following AAV injections, a single IP injection of ivermectin 10 mg/kg was administered to rats. The following day, rats were prepared for the thermal latency assay by shaving the fur off of their cheek, up to but not including the whisker pad. They were then placed in a 2.75 inch diameter×8 inch clear polycarbonate cylinder and allowed to acclimate to the environment. The rats naturally positioned themselves so their snout is just outside of the cylinder opening. Under video monitoring, the rats were then exposed to the beam of a 2W, infrared (808 nm) laser illumination focused to project a target size of approximately 1 mm×0.5 mm on the shaved cheek area. The rats then responded to the heat generated by the laser with an abrupt head position change (withdrawal reflex). The sequence was then repeated up to 10 trials on each side. The latency between the laser illumination onset and the head withdrawal reflex was measured from the video with video editing software allowing for frame by frame analysis.

### Results

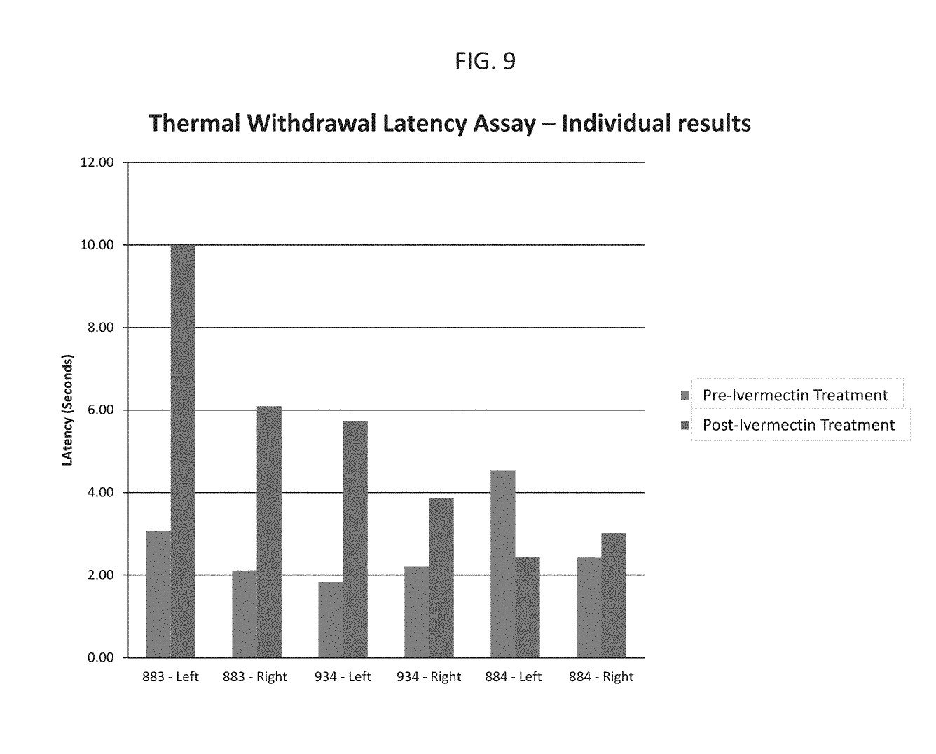

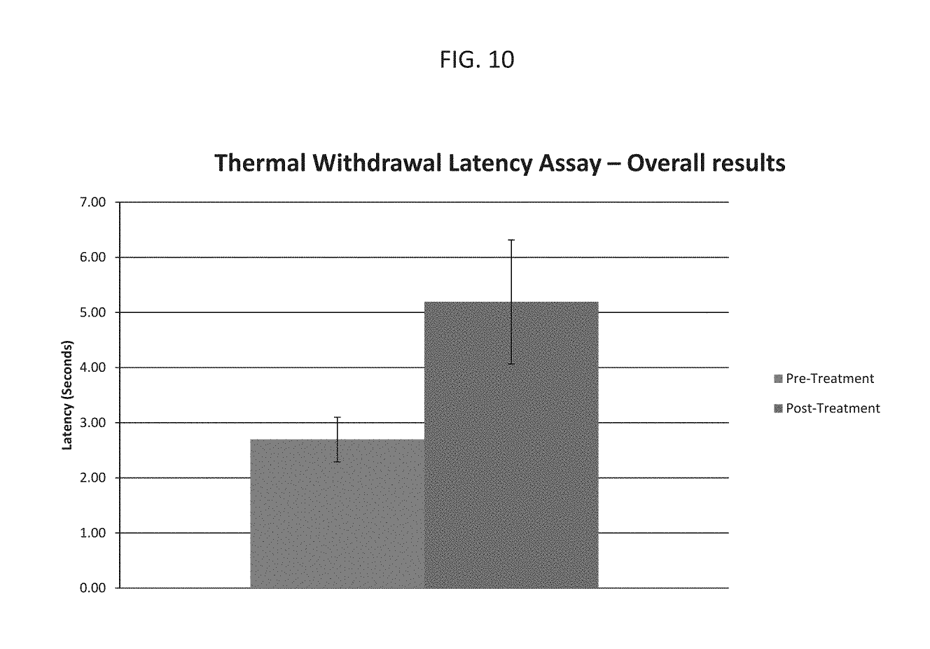

Individual subject results are shown in FIG. 9, with average results shown in FIG. 10. The average thermal withdrawal latency of test rats pre-treatment with ivermectin was 2.69 seconds. Following IP administration of ivermectin (10 mg/kg), average withdrawal latency was determined to be 5.19 seconds, paired t-test p value=0.054. Ivermectin treatment therefore resulted in a 92.9% hypoalgesic threshold shift in thermal pain sensitivity.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety to the same extent as if each individual publication, patent or patent application was specifically and individually indicated to be incorporated by reference. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1.-267. (canceled)

268. A method for treating pain in a subject having pain, the method comprising:
administering a small molecule drug that activates a ligand-gated ion channel (LGIC) in a peripheral neuron to the subject, wherein the subject heterologously expresses in a peripheral neuron a polynucleotide encoding one or more subunits of said ligand-gated ion channel (LGIC), wherein the pain in the subject is reduced.

269. The method according to claim 268, where said one or more subunits of said LGIC is a subunit of a glycine receptor (GlyR) or a mutein thereof.

270. The method according to claim 269, wherein said GlyR subunit is a GlyRα1 subunit mutein that comprises one or more amino acid substitutions selected from F207A and A288G and said drug is an avermectin.

271. The method according to claim 268, wherein said polynucleotide encoding said one or more subunits of a LGIC is operably linked to a promoter selected from the group consisting of a synapsin promoter, a TRPV1 promoter, a Nav1.7 promoter, a Nav1.8 promoter, a Nav1.9 promoter, a CamKII promoter, a NSE promoter, and an Advillin promoter.

272. The method according to claim 268, wherein said drug is administered to said subject at least one week after delivery of said nucleic acid molecule encoding said LGIC.

273. The method according to claim 268, wherein said drug is administered to said subject daily for at least three consecutive days.

274. The method according to claim 268, where the pain is measured in said subject one day after the administering of drug, wherein the pain is reduced.

275. A method for treating pain in a subject having pain, the method comprising:
administering to the subject a polynucleotide that encodes one or more subunits of a ligand-gated ion channel (LGIC), and
administering to said subject a small molecule drug that activates said LGIC in an amount effective to alleviate said pain after said administering of drug.

276. The method according to claim 275, wherein the polynucleotide is delivered by an AAV.

277. The method according to claim 276, wherein said AAV is delivered by intraganglionic or intraspinal injection.

278. The method according to claim 275, where said subunits of said LGIC is a subunit of a glycine receptor (GlyR) or a mutein thereof.

279. The method according to claim 278, wherein said GlyR subunit is a GlyRα1 subunit mutein that comprises one or more amino acid substitutions selected from F207A and A288G and said drug is an avermectin.

280. The method according to claim 275, wherein said polynucleotide encoding said one or more subunits of LGIC is operably linked to a promoter selected from the group consisting of a synapsin promoter, a TRPV1 promoter, a Nav1.7 promoter, a Nav1.8 promoter, a Nav1.9 promoter, a CamKII promoter, a NSE promoter, and an Advillin promoter.

281. The method according to claim 275, wherein said drug is administered to said subject at least one week after delivery of said polynucleotide encoding said one or more subunits of LGIC.

282. A composition for treating a neurological disease, the composition comprising: an AAV comprising a polynucleotide encoding one or more subunits of a ligand gated ion channel (LGIC) subunit operably linked to a promoter which is active in a peripheral neuron.

283. The composition according to claim 282, where said LGIC subunit is a subunits of a glycine receptor (GlyR) or a mutein thereof.

284. The composition according to claim 283, wherein said GlyR subunit is a GlyRα1 subunit mutein that comprises one or more amino acid substitutions selected from F207A and A288G.

285. The composition according to claim 282, where said promoter is selected from the group consisting of a synapsin promoter, a TRPV1 promoter, a Nav1.7 promoter, a Nav1.8 promoter, a Nav1.9 promoter, a CamKII promoter, a NSE promoter, and an Advillin promoter.

286. The composition according to claim 282, wherein said AAV is selected from the group consisting of AAV5 or a variant thereof, AAV6 or a variant thereof, AAV8 or a variant thereof, and AAV9 or a variant thereof.
